(19) United States
(12) Patent Application Publication
(10) Pub. No.: US 2026/0234258 A1
(43) Pub. Date: Aug. 13, 2026

(54) CD8 T CELL TARGETED IL2

(71) Applicant: REPERTOIRE IMMUNE MEDICINES, INC., Cambridge, MA (US)

(72) Inventors: Selvi RAMASAMY, Cambridge, MA (US); Stefano Vincenzo GULLA, Sudbury, MA (US); Jonathan D. NARDOZZI, Watertown, MA (US); Charles Christopher STUTZ, Cambridge, MA (US); Joanna Feltham SWAIN, Concord, MA (US)

(21) Appl. No.: 19/122,681

(22) PCT Filed: Oct. 20, 2023
(86) PCT No.: PCT/US2023/077472

§ 371 Date: Apr. 18, 2025

Related U.S. Application Data
(60) Provisional application No. 63/380,366, filed on Oct. 20, 2022.
(60) Provisional application No. 63/380,367, filed on Oct. 20, 2022.
(60) Provisional application No. 63/380,370, filed on Oct. 20, 2022.
(60) Provisional application No. 63/480,876, filed on Jan. 20, 2023.

(51) Int. Cl. A61K 38/00

(52) U.S. Cl. CPC C07K 16/2815; A61P 35/00; C07K 14/55; A61K 2039/505; C07K 2317/55; C07K 2317/565; C07K 2317/74; C07K 2317/92; C07K 2319/30; C07K 2319/33; C07K 2319/74

(57) ABSTRACT

According to various aspects of this disclosure, the present disclosure relates a fusion protein comprising i) an antigen-binding portion that binds CD8; and ii) a portion having an IL-2 activity (“IL-2 portion”).

This PCT application claims the priority benefit of U.S. Provisional Application No. 63/380,366, filed Oct. 20, 2022, U.S. Provisional Application No. 63/480,876, filed Jan. 20, 2023, U.S. Provisional Application No. 63/380,370, filed Oct. 20, 2022, and U.S. Provisional Application No. 63/380,367, filed Oct. 20, 2022, each of which are herein incorporated by reference in their entirety.

### REFERENCE TO A SEQUENCE LISTING SUBMITTED ELECTRONICALLY

The content of the electronically submitted sequence listing (Name: 4985_007PC02_Sequencelisting_ST26; Size: 188,925 bytes; and Date of Creation: Oct. 19, 2023) is herein incorporated by reference in its entirety.

### FIELD OF THE INVENTION

According to various aspects of this disclosure, the present disclosure relates to anti-CD8 antibody-IL2 fusion proteins.

### BACKGROUND OF THE INVENTION

Interleukin-2 (IL2 or IL-2) is a biologic cytokine that regulates key aspects of the immune system. IL2 has been used in attempts to boost immune responses in patients with cancer, inflammatory disease, or an autoimmune disease. IL2 is a potent T cells growth factor that promotes immune responses, including clonal expansion of antigen-activated T cells, drives development of CD4+T-helper (Th)1 and Th2 cells, terminally differentiates CD8+ cytotoxic T lymphocytes (CTLs), and opposes development of CD4+ Th17 and T-follicular helper (Tfh) cells. IL2 also shapes T cell memory recall responses.

Clinical trials have harnessed the T cell activating properties of IL2 in patients with cancer and HIV/AIDS by infusion of high doses of IL2 (typically >500,000 units/kg, repeatedly) to boost T and NK cells. Indeed, IL2 was approved by the FDA for use in patients with melanoma and renal cell carcinoma as some (approximately 5%) exhibited complete remissions. Nevertheless, response rates were low in these and other cancers while this therapy was accompanied by severe toxicity.

One of the central challenges in turning IL2 into a safe and effective anti-cancer immunotherapy is selectively delivering IL2 to CD8+ T cells without activating other undesired IL2R positive immune cell types (Tregs, NK, endothelial and ILC). Therefore, a need remains for selective targeting of IL2 that can be incorporated into a cancer therapy.

### BRIEF SUMMARY OF THE INVENTION

The present disclosure provides a fusion protein comprising i) an antigen-binding portion that binds CD8; and ii) a portion having an IL-2 activity (“IL-2 portion”). In some aspects, the fusion protein has a structure:
a) which comprises i) an antibody that binds CD8 comprising two heavy chains and two light chains in the antigen-binding portion and ii) two IL-2 proteins in the IL-2 portion, in some aspects, each of the IL-2 proteins in the IL-2 portion is fused to one of the C-termini of the two light chains;
b) which comprises i) an antibody that binds CD8 comprising two heavy chains and two light chains in the antigen-binding portion and ii) one IL-2 protein in the IL-2 portion, in some aspects, the IL-2 protein is fused to one of the C-termini of the two heavy chains;
c) which comprises i) an antibody that binds CD8 comprising two heavy chains and two light chains in the antigen-binding portion and ii) two IL-2 proteins in the IL-2 portion, in some aspects, each of the IL-2 proteins is fused to one of the N-termini of the two light chains;
d) which comprises i) an antibody that binds CD8 comprising two heavy chains and two light chains in the antigen-binding portion and ii) two IL-2 proteins in the IL-2 portion, in some aspects, each of the IL-2 proteins is fused to one of the N-termini of the heavy chains;
e) which comprises i) an antibody that binds CD8 comprising two heavy chains and two light chains in the antigen-binding portion and ii) one IL-2 protein in the IL-2 portion, in some aspects, the IL-2 protein is fused to one of the N-termini of the heavy chains;
f) which comprises i) an antibody that binds CD8 comprising two heavy chains and two light chains in the antigen-binding portion and ii) two IL-2 proteins in the IL-2 portion, in some aspects, each of the IL-2 proteins is fused to one of the C-termini of the two heavy chains;
g) which comprises i) an Fab that binds to CD8 in the antigen-binding portion, in some aspects, the Fab comprises a heavy chain variable region and a heavy chain constant region and a light chain variable region and a light chain constant region and ii) one IL-2 protein in the IL-2 portion, wherein the IL-2 protein is fused to the C-termini of the light chain constant region or the C-termini of the heavy chain constant region; or
h) which comprises i) an Fab that binds to CD8 in the antigen-binding portion and an Fc and ii) one IL-2 protein in the IL-2 portion, in some aspects, the Fab comprises a heavy chain variable region and a heavy chain constant region and a light chain variable region and a light chain constant region, in some aspects, the IL-2 protein is fused to the C-termini of the light chain constant region or the C-termini of the heavy chain constant region.

In some aspects, provided herein is a fusion protein comprising i) an antigen-binding portion that binds CD8; and ii) a portion having an IL-2 activity (“IL-2 portion”). In some aspects, the antigen-binding portion comprises an antibody comprising two heavy chains and two light chains and the IL-2 portion comprises two IL-2 proteins, and in some aspects, each of the IL-2 proteins is fused to one of the C-termini of the two light chains.

In some aspects, the antigen-binding portion comprises an antibody comprising two heavy chains and two light chains and the IL-2 portion comprises one IL-2 protein, and wherein the IL-2 protein is fused to one of the C-termini of the two heavy chains.

In some aspects, the antigen-binding portion comprises an antibody which comprises two heavy chains and two light chains and the IL-2 portion comprises two IL-2 proteins and wherein each of the IL-2 proteins is fused to one of the N-termini of the two heavy chains.

In some aspects, the antigen-binding portion comprises an antibody which comprises two heavy chains and two light chains and the IL-2 portion comprises two IL-2 proteins and wherein each of the IL-2 proteins is fused to one of the C-termini of two heavy chains;

In some aspects, the antigen-binding portion comprises an Fab comprising a heavy chain variable region and a heavy chain constant region and a light chain variable region and a light chain constant region and the IL-2 portion comprises one IL-2 protein, wherein the IL-2 protein is fused to the C-termini of the light chain constant region or the C-termini of the heavy chain constant region.

In some aspects, the antigen-binding portion comprises an Fab and an Fc, wherein the Fab comprises a heavy chain variable region and a heavy chain constant region and a light chain variable region and a light chain constant region and the IL-2 portion comprises one IL-2 protein, wherein the IL-2 protein is fused to the C-termini of the light chain constant region or the C-termini of the heavy chain constant region.

In some aspects, the antigen-binding portion comprises an antibody which comprises two heavy chains and two light chains and the IL-2 portion comprises two IL-2 proteins and wherein each of the IL-2 proteins is fused to one of the N-termini of the light chains.

In some aspects, the antigen-binding portion comprises an antibody which comprises two heavy chains and two light chains and the IL-2 portion comprises one IL-2 protein and wherein the IL-2 protein is fused to one of the N-termini of the heavy chains.

In some aspects, the antigen binding portion that binds to CD8 comprises SEQ ID NOs: 5 (HC) and 6 (LC).

In some aspects, the antigen-binding portion that binds CD8 does not substantially increase or decrease an activity of a CD8 molecule upon binding to the CD8 molecule.

In some aspects, the antigen-binding portion cross-competes with a reference antibody to binding to a CD8 molecule, wherein the reference antibody comprises a heavy chain variable region comprising SEQ ID NO: 22 and a light chain variable region comprising SEQ ID NO: 23.

In some aspects, wherein the antigen-binding portion binds to the same CD8 epitope as a reference antibody, wherein the reference antibody comprises a heavy chain variable region comprising SEQ ID NO: 22 and a light chain variable region comprising SEQ ID NO: 23.

In some aspects, each of the heavy chains or heavy chain variable regions in the antigen-binding portion comprises complementarity determining regions HCDR1, HCDR2, and HCDR3.

In some aspects: HCDR1 comprises the amino acid sequence set forth in SEQ ID NO: 24 or 139, HCDR2 comprises the amino acid sequence set forth in SEQ ID NO: 25 or 140, and HCDR3 comprises the amino acid sequence set forth in SEQ ID NO: 26.

In some aspects, each of the light chains or light chain variable regions in the antigen-binding portion comprises complementarity determining regions LCDR1, LCDR2, and LCDR3.

In some aspects, LCDR1 comprises the amino acid sequences set forth in SEQ ID NO: 27, 48, or 37; LCDR2 comprises the amino acid sequences set forth in 1) SEQ ID NO: 28, 2) X₁ASX₂X₃X₄X₅, wherein X₁ is R or D, X₂ is H or N, X₃ is L or R, X₄ is Q or A, and X₅ is S or T, or 3) X₁ASX₂X₃X₄X₅, wherein X₁, X₂, X₃, X₄, and X₅ are R, H, L, Q, and S, respectively; or X₁, X₂, X₃, X₄, and X₅ are D, N, R, A, and T, respectively; LCDR3 comprises the amino acid sequences set forth in SEQ ID NO: 29.

In some aspects, provided herein is a fusion protein comprising i) an antibody that binds CD8 or an antigen-binding portion thereof comprising a heavy chain variable region or a light chain variable region; and ii) a protein having an IL-2 activity (“IL-2 portion”) comprising an IL-2 protein having a decreased binding to CD25 relative to wild-type IL-2. In some aspects, the heavy chain variable region comprises complementarity determining regions HCDR1, HCDR2, and HCDR3.

In some aspects: HCDR1 comprises the amino acid sequence of SEQ ID NO: 24 or 139, HCDR2 comprises the amino acid sequence of SEQ ID NO: 25 or 140, HCDR3 comprises the amino acid sequence of SEQ ID NO: 26;

In some aspects, the light chain variable region comprises complementarity determining regions LCDR1, LCDR2, and LCDR3.

In some aspects: LCDR1 comprises the amino acid sequence set forth in SEQ ID NO: 27, 48, or 37; LCDR2 comprises the amino acid sequence set forth in 1) SEQ ID NO: 28, 2) X₁ASX₂X₃X₄X₅, wherein X₁ is R or D, X₂ is H or N, X₃ is L or R, X₄ is Q or A, and X₅ is S or T, or 3) X₁ASX₂X₃X₄X₅, wherein X₁, X₂, X₃, X₄, and X₅ are R, H, L, Q, and S, respectively; or X₁, X₂, X₃, X₄, and X₅ are D, N, R, A, T, respectively; LCDR3 comprises the amino acid sequence of SEQ ID NO: 29.

In some aspects, the antigen-binding portion comprises a heavy chain variable domain (VH) comprising an amino acid sequence having at least 95% sequence identity to SEQ ID NO: 22.

In some aspects, the antigen-binding portion comprises a light chain variable domain (VL) comprising an amino acid sequence having at least 95% sequence identity to SEQ ID NO: 23.

In some aspects, the antigen-binding portion comprises a heavy chain variable domain (VH) comprising the amino acid sequence set forth in SEQ ID NO: 22.

In some aspects, the antigen-binding portion comprises a light chain variable domain (VL) comprising the amino acid sequence set forth in SEQ ID NO: 23.

In some aspects, each of the heavy chains comprises the amino acid sequence set forth in SEQ ID NO: 5. In some aspects, each of the light chains comprises the amino acid sequence set forth in SEQ ID NO: 6.

In some aspects, the LCDR2 comprises the amino acid sequence set forth in X₁ASX₂X₃X₄X₅, wherein X₁, X₂, X₃, X₄, and X₅ are R, H, L, Q, and S, respectively; or X₁, X₂, X₃, X₄, and X₅ are D, N, R, A, and T, respectively.

In some aspects, the HCDR1, HCDR2, HCDR3, LCDR1, LCDR2, and LCDR3 comprise the amino acid sequences set forth in SEQ ID NOs: 139, 140, 26, 48, 28, and 29, respectively. In some aspects, the HCDR1, HCDR2, HCDR3, LCDR1, LCDR2, and LCDR3 comprise the amino acid sequences set forth in SEQ ID NOs: 24, 25, 26, 27, 28, or 29, respectively.

In some aspects, the VH and the VL comprise amino acid sequences having at least 95% sequence identity to SEQ ID NOs: 22 and 47; 39 and 47; 40 and 47; 41 and 47; or 42 and 47, respectively.

In some aspects, the HCDR1, HCDR2, HCDR3, LCDR1, LCDR2, and LCDR3 comprise the amino acid sequences set forth in SEQ ID NOs: 139, 140, 26, 27, 28, and 29, respectively.

In some aspects, the VH and the VL comprise amino acid sequences having at least 95% sequence identity to SEQ ID NOs: 39 and 45; 40 and 45; 41 and 45; 42 and 45; 22 and 45; 39 and 46; 40 and 46; 41 and 46; 42 and 46; 22 and 46; 39 and 23; 40 and 23; 41 and 23; 42 and 23; or 22 and 23, respectively.

In some aspects, the VH and the VL comprise amino acid sequences having at least 95% sequence identity to SEQ ID NOs: 30 and 31, respectively, or 33 and 31, respectively. In some aspects, the HCDR1, HCDR2, HCDR3, LCDR1, LCDR2, and LCDR3 comprise the amino acid sequences set forth in SEQ ID NOs: 139, 140, 26, 27, 50, and 29, respectively.

In some aspects, the VH and the VL comprise amino acid sequences having at least 95% sequence identity to SEQ ID NOs: 39 and 49; 40 and 49; 41 and 49; 42 and 49; or 22 and 49, respectively.

In some aspects, at least one of the IL-2 protein comprises an amino acid sequence having at least 90% sequence identity to SEQ ID NO: 110.

In some aspects, at least one of the IL-2 proteins has an amino acid that is different from the wild-type IL-2. In some aspects, the IL-2 protein has altered binding to at least one IL-2 receptor relative to wild-type IL-2. In some aspects, the IL-2 receptor is selected from the group consisting of CD25, CD122, and CD132. In some aspects, at least one of the IL-2 protein exhibits a decreased binding to CD25, relative to the wild-type IL-2. In some aspects, the at least one of the IL-2 protein comprises an amino acid that is different from the wild-type IL-2. In some aspects, the IL-2 protein has a decreased binding to CD25 (“substituted amino acid”). In some aspects, the substituted amino acid is at residues 34 to 45, residues 60 to 72, and/or residues 104 to 115 corresponding to SEQ ID NO: 110 (wild type IL2). In some aspects, the substituted amino acid is at R38, K35, F42, K43, Y45, E61, E62, P65, L72, or any combination thereof, corresponding to SEQ ID NO: 110. In some aspects, the substituted amino acid is at P34, K35, L36, T37, R38, L40, T41, F42, K43, Y45, E60, E61, K64, P65, E68, L72, M104, Y107, A108, D109, T111, T113, V115, or any combination thereof, corresponding to SEQ ID NO: 110. In some aspects, the substituted amino acid is at residues K35, L36, T37, K64, or any combination thereof, corresponding to SEQ ID NO: 110. In some aspects, the substituted amino acid is at residues K35, L36, T37, K64, P34, L40, T41, K43, Y45, K64, M104, Y107, T113, V115, or any combination thereof, corresponding to SEQ ID NO: 110. In some aspects, the substituted amino acid is at residues K35, L36, T37, K64, P34, L40, T41, K43, Y45, K64, M104, Y107, T113, V115, R38, F42, P65, E68, L72, or any combination thereof, corresponding to SEQ ID NO: 110. In some aspects, the substituted amino acid is at residues K35, L36, T37, K64, P34, L40, T41, K43, Y45, K64, M104, Y107, T113, V115, R38, F42, P65, E68, L72, L40, E61, A108, D109, T111, or any combination thereof, corresponding to SEQ ID NO: 110.

In some aspects, the substituted amino acid is E at residue 34 (P34E), E at residue 35 (K35E), N at residue 35 (K35N), W at residue 36 (L36W), A at residue 36 (L36A), N at residue 36 (L36N), E at residue 37 (T37E), T at residue 38 (R38T), F at residue 38 (R38F), H at residue 38 (R38H), W at residue 40 (L40W), G at residue 40 (L40G), D at residue 40 (L40D), A at residue 41 (T41A), Q at residue 41 (T41Q), E at residue 41 (T41E), Y at residue 42 (F42Y), S at residue 43 (K43S), R at residue 43 (K43R), Q at residue 43 (K43Q), L at residue 45 (Y45L), G at residue 45 (Y45G), F at residue 45 (Y45F), R at residue 60 (E60R), N at residue 61 (E61N), G at residue 61 (E61G), Y at residue 61 (E61Y), G at residue 64 (K64G), D at residue 64 (K64D), A at residue 64 (K64A), S at residue 65 (P65S), D at residue 65 (P65D), Y at residue 68 (E68Y), A at residue 68 (E68A), T at residue 68 (E68T), T at residue 72 (L72T), Q at residue 72 (L72Q), W at residue 72 (L72W), K at residue 104 (M104K), F at residue 107 (Y107F), L at residue 107 (Y107L), Q at residue 107 (Y107Q), H at residue 107 (Y107H), Y at residue 108 (A108Y), R at residue 109 (D109R), L at residue 111 (T111L), Q at residue 111 (T111Q), R at residue 111 (T111R), G at residue 113 (T113G), E at residue 113 (T113E), Y at residue 115 (V115Y), H at residue 115 (V115H), or any combination thereof corresponding to SEQ ID NO: 110.

In some aspects, the substituted amino acid is A at residue 38 (R38A), K at residue 38 (R38K), A at residue 35 (K35A), A at residue 42 (F42A), I at residue 42 (F42I), Q at residue 42 (F42Q), K at residue 42 (F42K), A at residue 43 (K43A), E at residue 43 (K43E), N at residue 43 (K43N), A at residue 45 (Y45A), S at residue 45 (Y45S), N at residue 45 (Y45N), E at residue 45 (Y45E), R at residue 45 (Y45R), Q at residue 61 (E61Q), A at residue 61 (E61A), K at residue 61 (E61K), A at residue 62 (E62A), K at residue 62 (E62K), L at residue 62 (E62L), V at residue 62 (E62V), L at residue 65 (P65L), H at residue 65 (P65H), G at residue 72 (L72G), Q at residue 72 (L72Q), or any combination thereof corresponding to SEQ ID NO: 110. In some aspects, the substituted amino acid is K35E, L36A, L36N, T37E, K64G, or any combination thereof corresponding to SEQ ID NO: 110. In some aspects, the substituted amino acid is K35E, L36A, L36N, T37E, K64G, P34E, L36W, L40D, T41A, K43Q, Y45L, K64D, M104K, Y107F, T113G, V115H, or any combination thereof corresponding to SEQ ID NO: 110. In some aspects, the substituted amino acid is K35E, L36A, L36N, T37E, K64G, P34E, L36W, L40D, T41A, K43Q, Y45L, K64D, M104K, Y107F, T113G, V115H, K35N, R38T, R38F, R38H, T41Q, F42Y, K43S, K43R, Y45F, K64A, P65S, P65D, E68A, L72W, V115Y, or any combination thereof corresponding to SEQ ID NO: 110. In some aspects, the substituted amino acid is K35E, L36A, L36N, T37E, K64G, P34E, L36W, L40D, T41A, K43Q, Y45L, K64D, M104K, Y107F, T113G, V115H, K35N, R38T, R38F, R38H, T41Q, F42Y, K43 S, K43R, Y45F, K64A, P65S, P65D, E68A, L72W, V115Y, L40W, T41E, Y45G, E61N, E61G, E68Y, E68T, L72T, A108Y, D109R, T111L, T111Q, or any combination thereof corresponding to SEQ ID NO: 110.

In some aspects, the IL-2 portion is linked to the antigen-binding portion by a linker. In some aspects, the linker useful for the fusion protein comprises the amino acid sequence set forth in SEQ ID NO: 107.

In some aspects, the Fc region is modified. In some aspects, the fusion protein of the present disclosure comprises two polypeptide chains. In some aspects, the first polypeptide chain comprises the amino acid sequence set forth in SEQ ID NO: 8 and the second polypeptide chain comprises the amino acid sequence set forth in SEQ ID NO: 10. In some aspects, the first polypeptide chain comprises the amino acid sequence set forth in SEQ ID NO: 3 and the second polypeptide chain comprises the amino acid sequence set forth in SEQ ID NO: 17. In some aspects, the first polypeptide chain comprises the amino acid sequence set forth in SEQ ID NO: 5 and the second polypeptide chain comprises the amino acid sequence set forth in SEQ ID NO: 20. In some aspects, the first polypeptide chain comprises the amino acid sequence set forth in SEQ ID NO: 1 and the second polypeptide chain comprises the amino acid sequence set forth in SEQ ID NO: 10.

In some aspects, the first polypeptide chain comprises the amino acid sequence set forth in SEQ ID NO: 7, the second polypeptide chain comprises the amino acid sequence set forth in SEQ ID NO: 125, and the third polypeptide chain comprises the amino acid sequence set forth in SEQ ID NO: 10. In some aspects, the first polypeptide chain comprises the amino acid sequence set forth in SEQ ID NO: 9 and the second polypeptide chain comprises the amino acid sequence set forth in SEQ ID NO: 2. In some aspects, the first polypeptide chain comprises the amino acid sequence set forth in SEQ ID NO: 13 and the second polypeptide chain comprises the amino acid sequence set forth in SEQ ID NO: 4. In some aspects, the first polypeptide chain comprises the amino acid sequence set forth in SEQ ID NO: 12 and the second polypeptide chain comprises the amino acid sequence set forth in SEQ ID NO: 18. In some aspects, the first polypeptide chain comprises the amino acid sequence set forth in SEQ ID NO: 111 and the second polypeptide chain comprises the amino acid sequence set forth in SEQ ID NO: 112. In some aspects, the first polypeptide chain comprises the amino acid sequence set forth in SEQ ID NO: 116 and the second polypeptide chain comprises the amino acid sequence set forth in SEQ ID NO: 117. In some aspects, the first polypeptide chain comprises the amino acid sequence set forth in SEQ ID NO: 112 and the second polypeptide chain comprises the amino acid sequence set forth in SEQ ID NO: 115. In some aspects, the first polypeptide chain comprises the amino acid sequence set forth in SEQ ID NO: 120 and the second polypeptide chain comprises the amino acid sequence set forth in SEQ ID NO: 117. In some aspects, the first polypeptide chain comprises the amino acid sequence set forth in SEQ ID NO: 121 and the second polypeptide chain comprises the amino acid sequence set forth in SEQ ID NO: 115. In some aspects, the first polypeptide chain comprises the amino acid sequence set forth in SEQ ID NO: 123 and the second polypeptide chain comprises the amino acid sequence set forth in SEQ ID NO: 117.

In some aspects, the fusion protein of the present disclosure comprises three polypeptide chains. In some aspects, the first polypeptide chain comprises the amino acid sequence set forth in SEQ ID NO: 11, the second polypeptide chain comprises the amino acid sequence set forth in SEQ ID NO: 15, and the third polypeptide chain comprises the amino acid sequence set forth in SEQ ID NO: 4. In some aspects, the first polypeptide chain comprises the amino acid sequence set forth in SEQ ID NO: 21, the second polypeptide chain comprises the amino acid sequence set forth in SEQ ID NO: 19 and the third polypeptide chain comprises the amino acid sequence set forth in SEQ ID NO: 6. In some aspects, the first polypeptide chain comprises the amino acid sequence set forth in SEQ ID NO: 14, the second polypeptide chain comprises the amino acid sequence set forth in SEQ ID NO: 16, and the third polypeptide chain comprises the amino acid sequence set forth in SEQ ID NO: 4. In some aspects, the first polypeptide chain comprises the amino acid sequence set forth in SEQ ID NO: 112, the second polypeptide chain comprises the amino acid sequence set forth in SEQ ID NO: 113, and the third polypeptide chain comprises the amino acid sequence set forth in SEQ ID NO: 114. In some aspects, the first polypeptide chain comprises the amino acid sequence set forth in SEQ ID NO: 117, the second polypeptide chain comprises the amino acid sequence set forth in SEQ ID NO: 118, and the third polypeptide chain comprises the amino acid sequence set forth in SEQ ID NO: 119. In some aspects, the first polypeptide chain comprises the amino acid sequence set forth in SEQ ID NO: 122, the second polypeptide chain comprises the amino acid sequence set forth in SEQ ID NO: 113, and the third polypeptide chain comprises the amino acid sequence set forth in SEQ ID NO: 117. In some aspects, the first polypeptide chain comprises the amino acid sequence set forth in SEQ ID NO: 114, the second polypeptide chain comprises the amino acid sequence set forth in SEQ ID NO: 117, and the third polypeptide chain comprises the amino acid sequence set forth in SEQ ID NO: 118. In some aspects, the first polypeptide chain comprises the amino acid sequence set forth in SEQ ID NO: 124, the second polypeptide chain comprises the amino acid sequence set forth in SEQ ID NO: 113, and the third polypeptide chain comprises the amino acid sequence set forth in SEQ ID NO: 117.

In some aspects, the fusion protein of the present disclosure has one or more of the following properties: a) activates CD8+ T cells with 10-fold or greater potency as compared to activation of NK cells; b) activates pSTAT5 in CD8+ T cells at similar or greater potency than Tregs; or c) preferentially induces proliferation of CD8+ T cells over Tregs and NK cells.

In some aspects, provided herein is a nucleic acid encoding any of the fusion proteins disclosed herein. In some aspects, provided herein is a vector comprising the any of the nucleic acids disclosed herein. In some aspects, provided herein is a recombinant cell comprising any of the nucleic acids disclosed herein. In some aspects, provided herein is a pharmaceutical composition comprising any of the fusion proteins disclosed herein, any of the nucleic acids disclosed herein, any of the vectors disclosed herein, any of the recombinant cells disclosed herein, and a pharmaceutically acceptable carrier or excipient.

In some aspects, provided herein is a method of producing a fusion protein disclosed herein or a method comprising culturing a cell that comprises any of the nucleic acids disclosed herein under suitable conditions that allow expression of the fusion protein. In some aspects, the method further comprises isolating the fusion protein.

In some aspects, the method further comprises formulating the fusion protein with a pharmaceutically acceptable carrier or excipient.

In some aspects, provided herein is a method of preventing or treating a disease or condition in a subject in need thereof, the method comprising administering to the subject any of the fusion proteins disclosed herein, any of the nucleic acids disclosed herein, any of the vectors disclosed herein, or any of the recombinant cells disclosed herein. In some aspects, the disease or condition is a cancer or an infection. In some aspects, the cancer is a bladder cancer, breast cancer, uterine cancer, endometrial carcinoma, ovarian cancer, colorectal cancer, colon cancer, head and neck cancer, lung cancer, stomach cancer, germ cell cancer, bone cancer, squamous cell cancer, skin cancer, neoplasm of the central nervous system, lymphoma, leukemia, sarcoma, virus-related cancer, small-cell lung cancer, non-small cell lung cancer, gastrointestinal cancer, Hodgkin's or non-Hodgkin's lymphoma, pancreatic cancer, glioblastoma, glioma, cervical cancer, liver cancer, myeloma, salivary gland carcinoma, kidney cancer, basal cell carcinoma, melanoma, prostate cancer, vulval cancer, thyroid cancer, testicular cancer, esophageal cancer, and any combinations thereof.

In some aspects, provided herein is a method of killing a cancer cell, the method comprising contacting the cancer cell and a CD8+ T cell to any of the fusion proteins disclosed herein. In some aspects, the cancer cell is a bladder cancer cell, breast cancer cell, uterine cancer cell, endometrial carcinoma cell, ovarian cancer cell, colorectal cancer cell, colon cancer cell, head and neck cancer cell, lung cancer cell, stomach cancer cell, germ cell cancer cell, bone cancer cell, squamous cell cancer cell, skin cancer cell, neoplasm cell of the central nervous system, lymphoma cell, leukemia cell, sarcoma cell, virus-related cancer cell, small-cell lung cancer cell, non-small cell lung cancer cell, gastrointestinal cancer cell, Hodgkin's or non-Hodgkin's lymphoma cell, pancreatic cancer cell, glioblastoma cell, glioma cell, cervical cancer cell, liver cancer cell, myeloma cell, salivary gland carcinoma cell, kidney cancer cell, basal cell carcinoma cell, melanoma cell, prostate cancer cell, vulval cancer cell, thyroid cancer cell, testicular cancer cell, esophageal cancer cell, and any combinations thereof.

In some aspects, the method further comprises administering an anti-cancer agent to the subject. In some aspects, the anti-cancer agent comprises an immunomodulator, an adoptive cell therapy, a checkpoint inhibitor, a small molecule based anti-cancer therapy, an antibody based cancer therapy, a cancer vaccine, or a chemotherapeutic agent. In some aspects, the immunomodulator is an immunostimulator. In some aspects, the immunostimulator comprises an immunostimulatory cytokine, an agonist of a costimulatory molecule, and/or an inhibitor of an immune checkpoint protein. In some aspects, the immunostimulatory cytokine comprises IL-12, IL-15, IL-2, IL-6, IL-7, IL-18, IL-21, IL-23, IL-27, or any combinations thereof.

In some aspects, the immunomodulator comprises an immunosuppressor. In some aspects, the immunosuppressor comprises an immunosuppressive cytokine, an inhibitor of a costimulatory molecule, and/or an agonist or antagonist of an immune checkpoint protein. In some aspects, the immunomodulatory comprises a checkpoint inhibitor. In some aspects, the checkpoint inhibitor comprises a PD-1 antagonist, a PD-L1 antagonist, a LAG-3 antagonist, a TIM-3 antagonist, a CTLA-4 antagonist, a B7-H3 antagonist, a B7-H4 antagonist, a BTLA antagonist, a KIR antagonist, a VISTA antagonist, a TIGIT antagonist, an IDO antagonist, a Siglec-15 antagonist, a GAL-9 antagonist, an HVEM antagonist, or combinations thereof.

In some aspects, provided herein is a recombinant IL-2 protein comprising an amino acid sequence having an Interleukin-2 activity (“IL-2 polypeptide”), which comprises an amino acid that is different from the wild-type IL-2 (“substituted amino acid”), wherein the substituted amino acid is at residues 34 to 45, residues 60 to 72, and/or residues 104 to 115 corresponding to SEQ ID NO: 1 (wild-type IL-2), wherein the recombinant protein exhibits a decreased binding to CD25. In some aspects, the substituted amino acid is A at residue 38 (R38A), K at residue 38 (R38K), A at residue 35 (K35A), A at residue 42 (F42A), I at residue 42 (F42I), Q at residue 42 (F42Q), K at residue 42 (F42K), A at residue 43 (K43A), E at residue 43 (K43E), N at residue 43 (K43N), A at residue 45 (Y45A), S at residue 45 (Y45S), N at residue 45 (Y45N), E at residue 45 (Y45E), R at residue 45 (Y45R), Q at residue 61 (E61Q), A at residue 61 (E61A), K at residue 61 (E61K), A at residue 62 (E62A), K at residue 62 (E62K), L at residue 62 (E62L), V at residue 62 (E62V), L at residue 65 (P65L), H at residue 65 (P65H), G at residue 72 (L72G), or Q at residue 72 (L72Q). In some aspects, the substituted amino acid is at residues P34, K35, L36, T37, R38, L40, T41, F42, K43, Y45, E60, E61, K64, P65, E68, L72, M104, Y107, A108, D109, T111, T113, V115, or any combination thereof, corresponding to SEQ ID NO: 110.

In some aspects, provided herein is a recombinant IL-2 protein comprising an amino acid sequence having an Interleukin-2 activity (“IL-2 polypeptide”), which comprises an amino acid that is different from the wild-type IL-2 (“substituted amino acid”), wherein the substituted amino acid is at residues 30 to 48, residues 64 to 74, and/or residues 103 to 124 corresponding to SEQ ID NO: 110 (wild-type IL2), wherein the recombinant protein exhibits an increased binding to CD25. In some aspects, the substituted amino acid is at residues N30, Y31, P34, K35, T37, T41, M46, K48, K64, P65, L66, E67, E68, V69, N71, L72, A73, Q74, F103, 1114, R120, F124, or any combination thereof, corresponding to SEQ ID NO: 110.

In some aspects, provided herein is a nucleic acid encoding a recombinant IL-2 protein. In some aspects, provided herein is a vector comprising the one or more nucleic acids encoding a recombinant IL-2 protein. In some aspects, provided herein is a recombinant cell comprising one or more nucleic acids encoding a recombinant IL-2 protein or a vector comprising the one or more nucleic acids.

In some aspects, provided herein is a pharmaceutical composition comprising any of the recombinant IL-2 proteins disclosed herein, any of the nucleic acids disclosed herein, any of the vectors disclosed herein, any of the recombinant cells disclosed herein, and a pharmaceutically acceptable carrier or excipient.

In some aspects, provided herein is a method of producing a recombinant IL-2 protein, the method comprising culturing a cell that comprises any of the nucleic acids encoding an IL-2 protein disclosed herein, under suitable conditions that allow expression of the IL-2 protein.

In some aspects, provided herein is a method of preventing or treating a disease or condition in a subject in need thereof, the method comprising administering to the subject any of the recombinant IL-2 proteins disclosed herein, any of the nucleic acids encoding a recombinant IL-2 protein disclosed herein, any of vectors comprising any of the nucleic acids encoding a recombinant IL-2 protein disclosed herein, or any of the recombinant cells comprising any of the nucleic acids encoding a recombinant IL-2 protein disclosed herein. In some aspects, the disease or condition is a cancer or an infectious disease.

### DESCRIPTION OF FIGURES

FIGS. 1A-1B show the mechanism of action for untargeted IL2 (RSV-IL2) and CD8 targeted IL2 (CD8-IL2).

FIGS. 2A-2H show different formats for the CD8 targeted IL2 constructs. These formats include Fab IL2 (LC) (FIG. 2A), Fc IL2 (LC) (FIG. 2B), IgG IL2 (LC) (FIG. 2C), IgG IL2 (HC) (FIG. 2D), IgG IL2 1×(HC) (FIG. 2E), IgG IL2 (HN) (FIG. 2F), IgG IL2 (LN) (FIG. 2G), IgG IL2 1×(HN) (FIG. 2H), Fab IL2 (HC) (FIG. 2I), Fab-Fc IL2 (HC at Knob chain) (FIG. 2J), and Fab-Fc IL2 (HC at Hole chain) (FIG. 2K).

FIGS. 3A-3F show avidity based binding and IL2 loading by the CD8 targeted IL2 constructs. FIG. 3A depicts the CD8 antibody binding on mouse CD8 T cells when incubated with mouse splenocytes at various concentration of indicated CD8-IL2 molecules. FIG. 3B depicts the IL2 binding on mouse CD8 T cells when incubated with mouse splenocytes at various concentration of indicated CD8-IL2 molecules. FIGS. 3C-3F show that the CD8-IL2 molecules tested only load IL2 on CD8 T cells over NK, CD4, B cells, and NK-T cells. CD8-IL2 molecules tested include CD8Fab-IL2 (FIG. 3C), CD8Fc-IL2 (FIG. 3D), CD8IgG-IL2LC (FIG. 3E), and CD8 IgG-IL2HC (FIG. 3F). Mouse CD8 antibody clone YTS105 was used in this experiment.

FIGS. 4A-4D show activation of pSTAT5 through CD8 targeted IL2 in CD8 T cells (FIG. 4A), CD4(Foxp3−) cells (FIG. 4B), Tregs (CD4+Foxp3−) (FIG. 4C), and NK cells (FIG. 4D).

FIGS. 5A-5C show CD8 T cell expansion mediated by CD8 targeted IL2 in CD8 T cells (FIG. 5A), Tregs (FIG. 5B), and NK cells (FIG. 5C).

FIGS. 6A-6C show a comparison of IL2 when linked to N-terminal heavy chain v. C-terminal light chain. Binding (FIG. 6A), pSTAT5 activation in CD8 T cells (FIG. 6B), and pSTAT5 activation in Tregs (FIG. 6C) were analyzed.

FIGS. 7A-7H show CD8 T cell expansion in naïve mice. FIGS. 7A-7D show spleen CD8 (FIG. 7A), Tregs (FIG. 7B), NK cells (FIG. 7C) numbers and CD8/Tregs ratio (FIG. 7D) in naïve C57BL/6 mice 4 days after treatment of 2 mg/kg CD8 targeted IL2 (mCD8 IgG IL2 (LC)), untargeted IL2 (RSV IgG IL2 (LC)), or HBSS control. FIGS. 7E-7H show the blood CD8 (FIG. 7E), Tregs (FIG. 7F), NK cells (FIG. 7G) numbers and CD8/Tregs ratio (FIG. 7H) 4 days after treatment of 2 mg/kg CD8 targeted IL2 (mCD8 IgG IL2 (LC)), untargeted IL2 (RSV IgG IL2 (LC)) in naïve C57BL/6 mice, or HBSS control.

FIGS. 8A-8C show the change in IL6 (FIG. 8A), INFγ (FIG. 8B) and MCP-1 (FIG. 8C) blood serum cytokine levels 48 hours after intravenous dosing of untargeted IL2 and CD8 targeted IL2 in B16F10 tumor bearing mice.

FIGS. 9A-9D show a comparison of mCD8 IgG-IL2 (LC) and mCD8 Fab-IL2 (LC) in vivo. FIGS. 9A-9C show tumor growth inhibition of B16F10 tumor in response to treatment of 3 mg/kg mCD8 IgG IL2 (LC) (FIG. 9A, 9C) and mCD8 Fab IL2 (LC) (FIG. 9A, 9B). FIG. 9D depicts the CD8 T cell expansion in the blood of MC38 tumor bearing mice 4 days after dosing 2 mg/kg equal molar mCD8 IgG IL2 (LC) and mCD8 Fab IL2 (LC) molecule.

FIGS. 10A-10E show the dose dependent tumor growth inhibition (FIGS. 10A, 10C-10E) of mCD8 IgG IL2 (LC) in B16F10 tumors, effect of CD8-IL2 and untargeted IL2 on body weight (FIG. 10B) in the B16F10 tumor bearing mice. mCD8 IgG IL2 (LC) was administered at 1 mg/kg (FIG. 10D) and 3 mg/kg doses (FIG. 10E), with HBSS alone used as a control (FIG. 10C).

FIGS. 11A-11F show that CD8-IL2 molecules with avid CD8 binding shows improved safety. The efficacy of CD8-IL2 molecules with different binding affinities in B16F10 tumor model (FIG. 11A) are shown for HBSS alone (FIG. 11C), mCD8 IgG-IL2 (LC) (FIG. 11D), mCD8 IgG-IL2 (HC) (FIG. 11E), and mCD8 Fc-IL2 (LC) (FIG. 11F). FIG. 11B depicts the body weight change response to untargeted IL2 and CD8-IL2 molecule with different binding affinity.

FIG. 12 shows IL6, INFy, MCP-1, G CSF and IL10 blood serum cytokine levels 48 hours after intravenous dosing of untargeted IL2 (RSV IgG IL2 (LC) and CD8 targeted IL2 (mCD8 IgG IL2 (LC), mCD8 IgG IL2 (HC) and CD8 Fe IL2 (LC)) with different binding affinity in B16F10 tumor bearing mice.

FIG. 13 shows the IL2 loading and binding affinity of mCD8 IgG IL2 (LC) or mCD8 IgG IL2 1×(HC) on mouse CD8 T cells when incubated with mouse splenocytes at various concentrations.

FIG. 14 shows the potency pSTAT5 activation in CD8 T cells, Tregs and NK cells by mCD8 IgG IL2 (LC) and mCD8 IgG IL2 1×(HC) when co-cultured with mouse splenocytes.

FIG. 15 shows the potency Ki67 activation in CD8 T cells, Tregs and NK cells by mCD8 IgG IL2 (LC) and mCD8 IgG IL2 1×(HC) when co-cultured with mouse splenocytes.

FIGS. 16A-16B show the efficacy of mCD8 IgG IL2 (LC) and mCD8 IgG IL2 1× (HC) molecule in B16F10 (FIG. 16A) and MC38 (FIG. 16B) tumor models.

FIGS. 17A-17D show the percentage of CD8 T cells, Tregs and NK cell within the CD45+ve blood cells in MC38 tumor bearing mice 4 days after dosing 2 mg/kg of mCD8 IgG IL2 (LC) and mCD8 IgG IL2 1×(HC) molecules.

FIG. 18 shows the pharmacokinetic properties of mCD8 IgG IL2 (LC) and mCD8 IgG IL2 1×(HC) molecules in naïve C57BL/6 mice.

FIG. 19 shows the efficacy of mCD8 IgG IL2 (LC) in combination with anti-PD1 in B16F10 melanoma model.

FIG. 20 shows the mean change in tumor volume of B16F10 tumor in response to HBSS, rIL2 and mCD8 IgG IL2 (LC) in the mice adoptively transferred with Pmel-1 T cells.

FIGS. 21B-21C show human CD8 targeted IL2 molecules loads IL2 selectively on human CD8 T cells. FIG. 21A shows the human CD8 antibody loading on CD8 T cells when human PBMC co-cultured with various concentration of the indicated CD8-IL2 molecules. FIG. 21B (hCD8 IgG IL2 (LC)) and FIG. 21C (hCD8 IgG IL2 (HC)) show that human CD8-IL2 molecules only loads IL2 on CD8 T cells over NK, CD4 and NK-T cells.

FIG. 22 shows the potency of pSTAT5 activation in CD8 T cells by human CD8-IL2 molecules (hCD8 IgG IL2 (LC) and hCD8 IgG IL2 1×(HC)) molecules compared to format matched untargeted IL2.

FIG. 23 shows the potency of Ki67 activation in CD8 T, Tregs and NK cells by hCD8 IgG IL2 (LC) and hCD8 IgG IL2 1×(HC) compared to format matched untargeted IL2 when co-cultured with human PBMC.

FIG. 24 shows the expansion of CD8, NK and Tregs in human PBMC response to treatment of human CD8 targeted IL2 molecules (hCD8 IgG IL2 (LC) and hCD8 IgG IL2 1×(HC)) and untargeted IL2 molecules (RSV IgG IL2 (LC) and RSV IgG IL2 1×(HC)).

FIGS. 25A-25B shows the epitopes bound by anti-CD8 antibodies 02A01, 03A01, 05F10, 14E06, ch51-1, and MCD8.

### DETAILED DESCRIPTION OF THE INVENTION

The present disclosure provides an improved form of IL-2 that can delivered to its receptors without eliciting undesired immune responses, e.g., activation of Tregs, NK, endothelial and ILC, systemic immune response or toxicity, or any combination thereof. In some aspects, the present disclosure provides a selective delivery of an IL-2 construct to its target cell, e.g., immune cells, e.g., T cells. In some aspects, provided herein is a fusion protein comprising i) an antigen-binding portion that binds CD8; and ii) a portion having an IL-2 activity (“IL-2 portion”). In some aspects, the fusion protein has a structure which comprises i) an antibody that binds CD8 comprising two heavy chains and two light chains in the antigen-binding portion and ii) two IL-2 proteins in the IL-2 portion, wherein each of the IL-2 proteins is fused to one of the C-termini of the two light chains.

In some aspects, the fusion protein has a structure which comprises i) an antibody that binds CD8 comprising two heavy chains and two light chains in the antigen-binding portion and ii) one IL-2 protein in the IL-2 portion, wherein the IL-2 protein is fused to one of the C-termini of the two heavy chains.

In some aspects, the fusion protein has a structure which comprises i) an antibody that binds CD8 comprising two heavy chains and two light chains in the antigen-binding portion and ii) two IL-2 proteins in the IL-2 portion, wherein each of the IL-2 proteins is fused to one of the N-termini of the two light chains.

In some aspects, the fusion protein has a structure which comprises i) an antibody that binds CD8 comprising two heavy chains and two light chains in the antigen-binding portion and ii) two IL-2 proteins in the IL-2 portion, wherein each of the IL-2 proteins is fused to one of the C-termini of the two heavy chains.

In some aspects, the fusion protein has a structure which comprises i) an Fab that binds to CD8 in the antigen-binding portion, wherein the Fab comprises a heavy chain variable region and a heavy chain constant region and a light chain variable region and a light chain constant region and ii) one IL-2 protein in the IL-2 portion, wherein the IL-2 protein is fused to the C-termini of the light chain constant region or the C-termini of the heavy chain constant region.

In some aspects, the fusion protein has a structure which comprises i) an Fab that binds to CD8 in the antigen-binding portion and an Fc and ii) one IL-2 protein in the IL-2 portion, wherein the Fab comprises a heavy chain variable region and a heavy chain constant region and a light chain variable region and a light chain constant region, and wherein the IL-2 protein is fused to the C-termini of the light chain constant region or the C-termini of the heavy chain constant region.

In some aspects, the fusion protein has a structure which comprises i) an antigen-binding portion that binds CD8; and ii) a portion having an IL-2 activity (“IL-2 portion”), wherein the antigen-binding portion comprises an antibody which comprises two heavy chains and two light chains and the IL-2 portion comprises two IL-2 proteins and wherein each of the IL-2 proteins is fused to one of the N-termini of the heavy chains.

In some aspects, the fusion protein has a structure which comprises i) an antigen-binding portion that binds CD8; and ii) a portion having an IL-2 activity (“IL-2 portion”), wherein the antigen-binding portion comprises an antibody which comprises two heavy chains and two light chains and the IL-2 portion comprises one IL-2 protein and wherein the IL-2 protein is fused to one of the N-termini of the heavy chains.

In some aspects, provided herein is a fusion protein comprising i) an antibody that binds CD8 or an antigen-binding portion thereof comprising a heavy chain variable region or a light chain variable region; and ii) a protein having an IL-2 activity (“IL-2 portion”) comprising an IL-2 protein having a decreased binding to CD25 relative to wild-type IL-2. In some aspects, the heavy chain variable region comprises complementarity determining regions HCDR1, HCDR2, and HCDR3. In some aspects, HCDR1 comprises the amino acid sequence set forth in SEQ ID NO: 24; HCDR2 comprises the amino acid sequence set forth in SEQ ID NO: 25; and HCDR3 comprises the amino acid sequence set forth in SEQ ID NO: 26. In some aspects, the light chain variable region comprises complementarity determining regions LCDR1, LCDR2, and LCDR3. In some aspects, LCDR1 comprises the amino acid sequence set forth in SEQ ID NO: 27; LCDR2 comprises the amino acid sequence set forth in SEQ ID NO: 28; and LCDR3 comprises the amino acid sequence set forth in SEQ ID NO: 29. In some aspects, LCDR1 comprises the amino acid sequence set forth in SEQ ID NO: 48; LCDR2 comprises the amino acid sequence set forth in SEQ ID NO: 28; and LCDR3 comprises the amino acid sequence set forth in SEQ ID NO: 29.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The terms “a” and “an” as used herein mean “one or more” and include the plural unless the context is inappropriate.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

As used herein, the expression “and/or” in connection with two or more recited objects includes individually each of the recited objects and the various combinations of two or more of the recited objects, unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Adeboye Adejare, Remington: The Science and Practice of Pharmacy (23d ed. 2020).

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

As used herein, the term “effective amount” refers to the amount of a compound (e.g., a compound of the present disclosure) sufficient to effect beneficial or desired results. An effective amount can be administered in one or more administrations, applications or dosages and is not intended to be limited to a particular formulation or administration route.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain aspects, the polypeptides can occur as single chains or associated chains.

The terms “administer,” “administering,” “administration,” and the like, as used herein, refer to methods that may be used to enable delivery of a drug, e.g., a fusion protein comprising a CD8 antigen binding site and an IL2 protein, to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the terms “subject” and “patient” are used interchangeably and refer to an organism to be treated by the methods and compositions described herein. Such organisms preferably include, but are not limited to, mammals (e.g., murines, simians, equines, bovines, porcines, canines, felines, and the like), and more preferably include humans.

It is understood that wherever aspects are described herein with the language “comprising,” otherwise analogous aspects described in terms of “consisting of” and/or “consisting essentially of” are also provided.

As used herein, the terms “derived from” or “derivative” refer to a component that is isolated from or made using a specified molecule, or information (e.g., a nucleic acid sequence) from the specified molecule. For example, a polynucleotide sequence that is derived from another polynucleotide sequence can include a polynucleotide sequence that is identical or substantially similar to the polynucleotide sequence it derives from. In the case of polynucleotides, the derived species can be obtained by, for example, naturally occurring mutagenesis, artificial directed mutagenesis, or artificial random mutagenesis. The mutagenesis used to derive polynucleotides can be intentionally directed or intentionally random, or a mixture of each. The mutagenesis of a polynucleotide to create a different polynucleotide derived from the first polynucleotide can be a random event (e.g., caused by polymerase infidelity) and the identification of the derived polynucleotide can be made by appropriate screening methods known in the art.

As used herein, the term “antigen-binding site” refers to an antigen-binding fragment of an immunoglobulin or a derivative or variant thereof that participates in antigen binding. For example, in human antibodies, an antigen-binding site is formed by the N-terminal variable domains of the heavy chain and light chain, which are also called “heavy chain variable domain (VH)” and “light chain variable domain (VL),” respectively. In each of the variable domains, three highly divergent stretches called “hypervariable regions” are interposed between more conserved flanking stretches known as “framework regions.” The three hypervariable regions of a VH and the three hypervariable regions of a VL are disposed relative to each other in three-dimensional space to form an antigen-binding surface complementary to the three-dimensional surface of a bound antigen. The hypervariable regions are also referred to as “complementarity-determining regions” or “CDRs.” Examples of antigen-binding fragments of an immunoglobulin, include, for example, Fab, Fab′, and F(ab′)2 fragments. Examples of variants of antigen-binding fragments of immunoglobulins include, for example, single chain antibodies or scFvs. Certain animals have different forms of antibodies. For example, camelids have antibodies comprising VHH fragments and cartilaginous fishes have antibodies called “new antigen receptor immunoglobulins” or “IgNARs” comprising VNAR fragments, where such fragments, which are single, monomeric antibody variable domains that are able to bind selectively to a specific antigen independently of another variable domain, are called “single domain antibody,” “sdAb,” or “nanobody.” An antigen-binding site can comprise either a pair of VH and VL or an sdAb. The antigen-binding site disclosed herein can be recombinant, chimeric, deimmunized, humanized, and/or affinity matured (see, e.g., U.S. Pat. No. 4,816,567; Morrison et al. (1984) Proc. Natl. Acad. Sci. U.S.A., 81: 6851-55; Morrison et al. (1985) Proc. Natl. Acad. Sci. U.S.A., 81:6851; Takeda et al. (1985) Nature, 314: 452).

As used herein, the term “CD8-binding site” refers to an antigen-binding site, as defined herein, that binds CD8 (e.g., human CD8).

As used herein, the term “binding agent” e.g., “CD8 binding agent” refers to a binding protein, e.g., an immunoglobulin or a derivative thereof (e.g., an antigen-binding fragment thereof, scFvs, bispecific antibodies, diabodies), or variant thereof, that contains an antigen-binding site that binds an antigen (e.g., CD8, e.g., human CD8). It is understood that a binding agent also includes states (e.g., forms or configurations) of the binding agent that do not bind to the target, which can be transformed or modified into states that bind the target. For example, a binding agent may also include a masking moiety that reduces the affinity of the binding agent to its target. When the masking moiety is removed (e.g., cleaved), the binding agent recovers its binding affinity to the target.

The term “silent,” as used herein in the context of a CD8 binding agent or a CD8-binding site, refers to a CD8 binding agent or a CD8-binding site that does not substantially change the function or activity of CD8 upon binding to the CD8. In certain embodiments, the CD8 binding agent or the CD8-binding site lacks substantial agonistic activity or substantial antagonistic activity upon binding CD8. In certain embodiments, the CD8 binding agent or the antigen-binding site does not increase an activity of CD8 by more than 10%, 20%, 30%, 40%, 50%, or 60% and/or does not decrease an activity of CD8 by more than 10%, 20%, 30%, 40%, 50%, or 60% In certain embodiments, the CD8 binding agent or the antigen-binding site does not increase an activity of CD8 by more than 1.1 fold, 1.2 fold, 1.3 fold, 1.4 fold, 1.5 fold, 1.6 fold, 1.7 fold, 1.8 fold, 1.9 fold, or 2 fold and/or the does not decrease an activity of CD8 by more than 1.1 fold, 1.2 fold, 1.3 fold, 1.4 fold, 1.5 fold, 1.6 fold, 1.7 fold, 1.8 fold, 1.9 fold, or 2 fold. The activity of CD8 can be stabilization of a peptide-MHC-TCR complex, activation of a TCR signaling pathway, and/or activation of a transcription factor downstream of TCR signaling, as measured by the assays disclosed herein (see, e.g., the Examples section below).

The term “cross-compete,” as used herein in the context of a subject antibody and a reference antibody, indicates that the subject antibody competes for binding to an antigen (e.g., CD8) with the reference antibody and vice versa. In an exemplary assay, a first anti-CD8 antibody is immobilized on a solid surface, CD8aP is bound to the first antibody, and binding of a second anti-CD8 antibody is assessed. If the second antibody does not generate a significant binding signal (e.g., resonance unit does not increase by at least 25%, 50%, or 2-fold), the second antibody competes with the first antibody for binding CD8 (see, e.g., Example 1 below). A subject antibody cross-competes with a reference antibody if competition is observed whether the reference antibody is used as the first antibody and the subject antibody is used as the second antibody, or the subject antibody is used as the first antibody and the reference antibody is used as the second antibody in this assay. A skilled artisan can select the concentrations of the antibodies used in the competition assays based on the affinities of the antibodies for the antigen and the valency of the antibodies. Exemplary assays are described in Cox et al., “Immunoassay Methods,” in ASSAY GUIDANCE MANUAL [INTERNET], Updated Dec. 24, 2014 (www.ncbi.nlm.nih.gov/books/NBK92434/; accessed Sep. 29, 2015); Silman et al. (2001) CYTOMETRY, 44: 30-37; and Finco et al. (2011) J. PHARM. BIOMED. ANAL., 54: 351-358. A CD8 binding agent comprising an antigen-binding site, such as a fragment of an anti-CD8 antibody, can also be assessed in this assay.

As used herein, the term “tumor-associated antigen” refers to any antigen, including but not limited to a protein, glycoprotein, ganglioside, carbohydrate, or lipid, that is expressed or otherwise present on the surface of a malignant cell, or present in the tumor microenvironment (e.g., on the surface of tumor-associated blood vessels, mesenchymal stroma, or immune infiltrates, or in an extracellular matrix).

The term “Cₘₐₓ” as used herein refers to the maximum plasma concentration of a fusion protein after administration of the fusion protein.

The term “Tₘₐₓ” as used herein refers to the time required to reach the maximal plasma concentration Cₘₐₓ after administration of a fusion protein.

The term “AUC” as used herein refers to the area under the curve of a plot of plasma concentration versus time following administration of a fusion protein.

The term “AUC₀₋₄” as used herein refers to the area under the fusion protein concentration-time curve from time zero to the time of the last measurable concentration (Ct).

### II. Fusion Proteins

In some aspects, provided herein is a fusion protein comprising i) an antigen-binding portion that binds CD8; and ii) a portion having an IL-2 activity (“IL-2 portion”). In some aspects, the antigen-binding portion that binds CD8 and IL-2 portion can be fused in any particular numbers or manner, e.g., two VHs and two VLs that binds CD8 and two IL-2 proteins.

In some aspects, the fusion protein comprises an anti-CD8 antibody and an IL-2 protein. In some aspects, the anti-CD8 antibody comprises i) a first heavy chain; ii) a second heavy chain; iii) a first light chain; and iv) a second heavy chain. In some aspects, at least one IL-2 protein is fused to i) the N terminus of the first heavy chain; ii) the N terminus of the second heavy chain; iii) the N terminus of the first light chain; iv) the N terminus of the second light chain; v) the C terminus of the first heavy chain; vi) the C terminus of the second heavy chain; vii) the C terminus of the first light chain; viii) the C terminus of the second light chain; or any combination thereof.

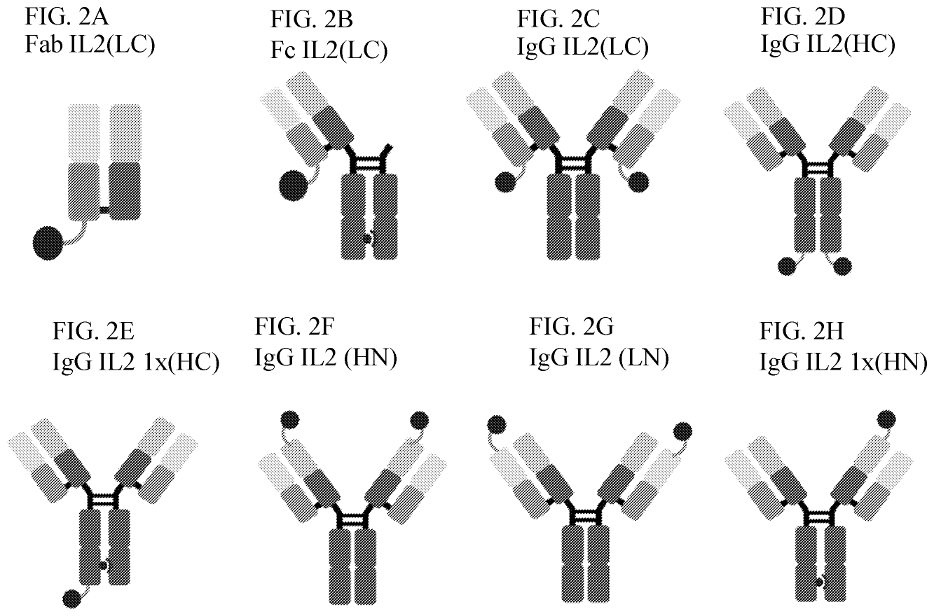

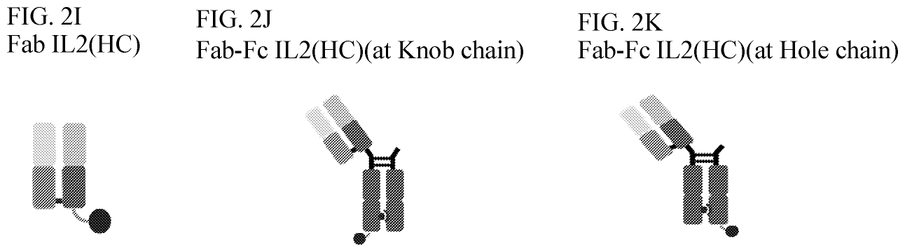

In some aspects, the fusion protein has a structure which comprises i) an antibody that binds CD8 comprising two heavy chains and two light chains in the antigen-binding portion and ii) two IL-2 proteins in the IL-2 portion, wherein each of the IL-2 proteins is fused to one of the C-termini of the two light chains. In some aspects, the fusion protein has the structure of FIG. 2C. In some aspects, the fusion protein comprises two first polypeptides, each comprising a heavy chain of an antibody fused to an IL-2 protein, and two second polypeptides, each comprising a light chain of an antibody, wherein the heavy chain and the light chain together binds to CD8. In some aspects, the two first polypeptides can be identical or different. In some aspects, the two second polypeptides can be identical or different.

In some aspects, the fusion protein has a structure which comprises i) an antibody that binds CD8 comprising two heavy chains and two light chains in the antigen-binding portion and ii) one IL-2 protein in the IL-2 portion, wherein the IL-2 protein is fused to one of the C-termini of the two heavy chains. In some aspects, the fusion protein has the structure of FIG. 2E. In some aspects, the fusion protein comprises a first polypeptide comprising a heavy chain of an antibody fused to an IL-2 protein at the C terminus of the heavy chain, a second polypeptide comprising a heavy chain of an antibody, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, the fusion protein has a structure which comprises i) an antibody that binds CD8 comprising two heavy chains and two light chains in the antigen-binding portion and ii) two IL-2 proteins in the IL-2 portion, wherein each of the IL-2 proteins is fused to one of the N-termini of the two light chains. In some aspect, the fusion protein has the structure of FIG. 2G. In some aspects, the fusion protein comprises two first polypeptides, each comprising an IL-2 protein fused to a light chain of an antibody at the N terminus of the light chain, and two second polypeptides, each comprising a heavy chain of an antibody, wherein the heavy chain and light chain can bind to CD8. In some aspects, the two first polypeptides are identical or different. In some aspects, the two second polypeptides are identical or different.

In some aspects, the fusion protein has a structure which comprises i) an antibody that binds CD8 comprising two heavy chains and two light chains in the antigen-binding portion and ii) two IL-2 proteins in the IL-2 portion, wherein each of the IL-2 proteins is fused to one of the N-termini of the heavy chains.

In some aspects, the fusion protein has the structure of FIG. 2F. In some aspects, the fusion protein comprises a first polypeptide comprising a heavy chain of an antibody fused to an IL-2 protein at the N terminus of the heavy chain, a second polypeptide comprising a heavy chain of an antibody fused to an IL-2 protein at the N terminus of the heavy chain, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, the fusion protein has a structure which comprises i) an antibody that binds CD8 comprising two heavy chains and two light chains in the antigen-binding portion and ii) one IL-2 protein in the IL-2 portion, wherein the IL-2 protein is fused to one of the N-termini of the heavy chains.

In some aspects, the fusion protein has the structure of FIG. 2H. In some aspects, the fusion protein comprises a first polypeptide comprising a heavy chain of an antibody fused to an IL-2 protein at the N terminus of the heavy chain, a second polypeptide comprising a heavy chain of a antibody, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, the fusion protein has a structure which comprises i) an antibody that binds CD8 comprising two heavy chains and two light chains in the antigen-binding portion and ii) two IL-2 proteins in the IL-2 portion, wherein each of the IL-2 proteins is fused to one of the C-termini of the two heavy chains.

In some aspects, the fusion protein has the structure of FIG. 2D. In some aspects, the fusion protein comprises a first polypeptide comprising a heavy chain of an antibody fused to an IL-2 protein at the C terminus of the heavy chain, a second polypeptide comprising a heavy chain of an antibody fused to an IL-2 protein at the C terminus of the heavy chain, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, the fusion protein has a structure which comprises i) an Fab that binds to CD8 in the antigen-binding portion, wherein the Fab comprises a heavy chain variable region and a heavy chain constant region and a light chain variable region and a light chain constant region and ii) one IL-2 protein in the IL-2 portion, wherein the IL-2 protein is fused to the C-termini of the light chain constant region or the C-termini of the heavy chain constant region.

In some aspects, the fusion protein has the structure of FIG. 2A. In some aspects, the fusion protein comprises a first polypeptide comprising a Fab comprising a heavy chain variable region, a heavy chain constant region, a light chain variable region. In some aspects, the IL-2 protein is fused to the C terminus of the light chain constant region. In some aspects, the IL-2 protein is fused to the C terminus of the heavy chain constant region. In some aspects, the heavy chain variable region and/or the light chain variable region can bind to CD8.

In some aspects, the fusion protein has a structure which comprises i) an Fab that binds to CD8 in the antigen-binding portion and an Fc and ii) one IL-2 protein in the IL-2 portion, wherein the Fab comprises a heavy chain variable region and a heavy chain constant region and a light chain variable region and a light chain constant region, and wherein the IL-2 protein is fused to the C-termini of the light chain constant region or the C-termini of the heavy chain constant region.

In some aspects, the fusion protein has the structure of FIG. 2B. In some aspects, the fusion protein comprises a first polypeptide comprising a Fab comprising a heavy chain variable region, a heavy chain constant region, a light chain variable region; and a second polypeptide comprising a Fc region. In some aspects, the IL-2 protein is fused to the C terminus of the light chain constant region. In some aspects, the IL-2 protein is fused to the C terminus of the heavy chain constant region. In some aspects, the heavy chain variable region and/or the light chain variable region can bind to CD8.

In some aspects, provided herein is a fusion protein comprising i) an antigen-binding portion that binds CD8; and ii) a portion having an IL-2 activity (“IL-2 portion”), wherein the antigen-binding portion comprises an antibody comprising two heavy chains and two light chains and the IL-2 portion comprises two IL-2 proteins and wherein each of the IL-2 proteins is fused to one of the C-termini of the two light chains.

In some aspects, the fusion protein has the structure of FIG. 2C. In some aspects, the fusion protein comprises two first polypeptides, each comprising a heavy chain of an antibody fused to an IL-2 protein, and two second polypeptides, each comprising a light chain of an antibody, wherein the heavy chain and the light chain together binds to CD8. In some aspects, the two first polypeptides can be identical or different. In some aspects, the two second polypeptides can be identical or different.

In some aspects, provided herein is a fusion protein comprising i) an antigen-binding portion that binds CD8; and ii) a portion having an IL-2 activity (“IL-2 portion”), wherein the antigen-binding portion comprises an antibody comprising two heavy chains and two light chains and the IL-2 portion comprises one IL-2 protein, and wherein the IL-2 protein is fused to one of the C-termini of the two heavy chains.

In some aspects, the fusion protein has the structure of FIG. 2E. In some aspects, the fusion protein comprises a first polypeptide comprising a heavy chain of an antibody fused to an IL-2 protein at the C terminus of the heavy chain, a second polypeptide comprising a heavy chain of an antibody, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, provided herein is a fusion protein comprising i) an antigen-binding portion that binds CD8; and ii) a portion having an IL-2 activity (“IL-2 portion”), wherein the antigen-binding portion comprises an antibody which comprises two heavy chains and two light chains and the IL-2 portion comprises two IL-2 proteins and wherein each of the IL-2 proteins is fused to one of the N-termini of the two heavy chains.

In some aspects, the fusion protein has the structure of FIG. 2F. In some aspects, the fusion protein comprises a first polypeptide comprising a heavy chain of an antibody fused to an IL-2 protein at the N terminus of the heavy chain, a second polypeptide comprising a heavy chain of an antibody fused to an IL-2 protein at the N terminus of the heavy chain, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, provided herein is a fusion protein comprising i) an antigen-binding portion that binds CD8; and ii) a portion having an IL-2 activity (“IL-2 portion”), wherein the antigen-binding portion comprises an antibody which comprises two heavy chains and two light chains and the IL-2 portion comprises one IL-2 protein and wherein the IL-2 protein is fused to one of the N-termini of two heavy chains.

In some aspects, the fusion protein has the structure of FIG. 2H. In some aspects, the fusion protein comprises a first polypeptide comprising a heavy chain of an antibody fused to an IL-2 protein at the N terminus of the heavy chain, a second polypeptide comprising a heavy chain of a antibody, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, provided herein is a fusion protein comprising i) an antigen-binding portion that binds CD8; and ii) a portion having an IL-2 activity (“IL-2 portion”), wherein the antigen-binding portion comprises an Fab comprising a heavy chain variable region and a heavy chain constant region and a light chain variable region and a light chain constant region and the IL-2 portion comprises one IL-2 protein, wherein the IL-2 protein is fused to the C-termini of the light chain constant region or the C-termini of the heavy chain constant region.

In some aspects, the fusion protein has the structure of FIG. 2A. In some aspects, the fusion protein comprises a first polypeptide comprising a Fab comprising a heavy chain variable region, a heavy chain constant region, a light chain variable region. In some aspects, the IL-2 protein is fused to the C terminus of the light chain constant region. In some aspects, the IL-2 protein is fused to the C terminus of the heavy chain constant region. In some aspects, the heavy chain variable region and/or the light chain variable region can bind to CD8.

In some aspects, provided herein is a fusion protein comprising i) an antigen-binding portion that binds CD8; and ii) a portion having an IL-2 activity (“IL-2 portion”), wherein the antigen binding portion comprises an Fab and an Fc, wherein the Fab comprises a heavy chain variable region and a heavy chain constant region and a light chain variable region and a light chain constant region and the IL-2 portion comprises one IL-2 protein, wherein the IL-2 protein is fused to the C-termini of the light chain constant region or the C-termini of the heavy chain constant region.

In some aspects, the fusion protein has the structure of FIG. 2B. In some aspects, the fusion protein comprises a first polypeptide comprising a Fab comprising a heavy chain variable region, a heavy chain constant region, a light chain variable region; and a second polypeptide comprising a Fc region. In some aspects, the IL-2 protein is fused to the C terminus of the light chain constant region. In some aspects, the IL-2 protein is fused to the C terminus of the heavy chain constant region. In some aspects, the heavy chain variable region and/or the light chain variable region can bind to CD8.

In some aspects, provided herein is a fusion protein comprising i) an antigen-binding portion that binds CD8; and ii) a portion having an IL-2 activity (“IL-2 portion”), wherein the antigen-binding portion comprises an antibody which comprises two heavy chains and two light chains and the IL-2 portion comprises two IL-2 proteins and wherein each of the IL-2 proteins is fused to one of the C-termini of the heavy chains.

In some aspects, the fusion protein has the structure of FIG. 2D. In some aspects, the fusion protein comprises a first polypeptide comprising a heavy chain of an antibody fused to an IL-2 protein at the C terminus of the heavy chain, a second polypeptide comprising a heavy chain of an antibody fused to an IL-2 protein at the C terminus of the heavy chain, and two third polypeptides, each comprising a light chain of an antibody, wherein the heavy chain in the first polypeptide or the second polypeptide and the light chain in the third polypeptides can bind to CD8. In some aspects, the heavy chains in the first and second polypeptides are identical or different. In some aspects, the two third polypeptides are identical or different.

In some aspects, provided herein is a fusion protein comprising i) an antigen-binding portion that binds CD8; and ii) a portion having an IL-2 activity (“IL-2 portion”), wherein the antigen-binding portion comprises an antibody which comprises two heavy chains and two light chains and the IL-2 portion comprises two IL-2 proteins and wherein each IL-2 protein is fused to one of the N-termini of the light chains.

In some aspect, the fusion protein has the structure of FIG. 2G. In some aspects, the fusion protein comprises two first polypeptides, each comprising an IL-2 protein fused to a light chain of an antibody at the N terminus of the light chain, and two second polypeptides, each comprising a heavy chain of an antibody, wherein the heavy chain and light chain can bind to CD8. In some aspects, the two first polypeptides are identical or different. In some aspects, the two second polypeptides are identical or different.

In some aspects, the antigen-binding portion that binds to CD8 comprises the amino acid sequence set forth in SEQ ID NOs: 5 (HC) and 6 (LC).

In some aspects, the antigen-binding portion that binds CD8 does not substantially increase or decrease an activity of a CD8 molecule upon binding to the CD8 molecule.

In some aspects, the antigen-binding portion cross-competes with a reference antibody to binding to a CD8 molecule, wherein the reference antibody comprises a heavy chain variable region comprising the amino acid sequence set forth in SEQ ID NO: 22 and a light chain variable region comprising the amino acid sequence set forth in SEQ ID NO: 23.

In some aspects, the antigen-binding portion binds to the same CD8 epitope as a reference antibody, wherein the reference antibody comprises a heavy chain variable region comprising the amino acid sequence set forth in SEQ ID NO: 22 and a light chain variable region comprising the amino acid sequence set forth in SEQ ID NO: 23.

In some aspects, each of the heavy chains or heavy chain variable regions in the antigen-binding portion comprises complementarity determining regions HCDR1, HCDR2, and HCDR3. In some aspects, HCDR1 comprises the amino acid sequence set forth in SEQ ID NO: 24. In some aspects, HCDR2 comprises the amino acid sequence set forth in SEQ ID NO: 25. In some aspects, and HCDR3 comprises the amino acid sequence set forth in SEQ ID NO: 26. In some aspects, each of the light chains or light chain variable regions in the antigen-binding portion comprises complementarity determining regions LCDR1, LCDR2, and LCDR3. In some aspects, LCDR1 comprises the amino acid sequence o set forth in f SEQ ID NO: 27. In some aspects, LCDR2 comprises the amino acid sequence set forth in SEQ ID NO: 28. In some aspects, LCDR3 comprises the amino acid sequence set forth in SEQ ID NO: 29. In some aspects, each of the light chains or light chain variable regions in the antigen-binding portion comprises complementarity determining regions LCDR1, LCDR2, and LCDR3. In some aspects, LCDR1 comprises the amino acid sequence set forth in SEQ ID NO: 48. In some aspects, LCDR2 comprises the amino acid sequence set forth in SEQ ID NO: 28. In some aspects, LCDR3 comprises the amino acid sequence set forth in SEQ ID NO: 29.

In some aspects, provided herein is a fusion protein comprising i) an antibody that binds CD8 or an antigen-binding portion thereof comprising a heavy chain variable region or a light chain variable region; and ii) a protein having an IL-2 activity (“IL-2 portion”) comprising an IL-2 protein having a decreased binding to CD25 relative to wild-type IL-2. In some aspects, the heavy chain variable region comprises complementarity determining regions HCDR1, HCDR2, and HCDR3. In some aspects, HCDR1 comprises the amino acid sequence set forth in SEQ ID NO: 24; HCDR2 comprises the amino acid sequence set forth in SEQ ID NO: 25; and HCDR3 comprises the amino acid sequence set forth in SEQ ID NO: 26. In some aspects, the light chain variable region comprises complementarity determining regions LCDR1, LCDR2, and LCDR3. In some aspects, LCDR1 comprises the amino acid sequence set forth in SEQ ID NO: 27; LCDR2 comprises the amino acid sequence set forth in SEQ ID NO: 28; and LCDR3 comprises the amino acid sequence set forth in SEQ ID NO: 29. In some aspects, LCDR1 comprises the amino acid sequence set forth in SEQ ID NO: 48; LCDR2 comprises the amino acid sequence set forth in SEQ ID NO: 28; and LCDR3 comprises the amino acid sequence set forth in SEQ ID NO: 29.

In some aspects, the antigen-binding portion comprises a heavy chain variable domain (VH) comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to the amino acid sequence set forth in SEQ ID NO: 22.

In some aspects, the antigen-binding portion comprises a heavy chain variable domain (VH) comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to the amino acid sequence set forth in SEQ ID NO: 22.

In some aspects, the antigen-binding portion comprises a light chain variable domain (VL) comprising an amino acid sequence at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 23.

In some aspects, the antigen-binding portion comprises a light chain variable domain (VL) comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to the amino acid sequence set forth in SEQ ID NO: 23.

In some aspects, the antigen-binding portion comprises a heavy chain variable domain (VH) comprising the amino acid sequence set forth in SEQ ID NO: 22. In some aspects, the antigen-binding portion comprises a light chain variable domain (VL) comprising the amino acid sequence set forth in SEQ ID NO: 23.

### A. Anti-CD8 Antibodies

In some aspects, the fusion protein disclosed herein comprises an antigen-binding site that binds CD8 (e.g., human CD8 and/or cynomolgus CD8). Tables 1A-B, 2A-B, and 3A-B below list VH, VL, and CDR sequences of the antigen-binding sites. In some aspects, the CDR sequences are identified under the Kabat numbering scheme (Tables 1A, 2A, and 3A) or Chothia numbering scheme (Tables 1B, 2B, and 3B). However, it is to be appreciated that the CDR sequences of the CD8 antigen-binding sites of the present disclosure can be identified under other algorithms in the art, such as Chothia or IMGT. In Tables 1A-1B, and Tables 2A-2B, any of the humanized VH sequences can pair with any of the humanized VL sequences in the same table to form a CD8-binding site.

TABLE 1A
Exemplary CD8 antigen-binding sites derived from 02A01 (Kabat)

| | Heavy Chain Variable Domain | Light Chain Variable Domain |
|---|---|---|
| Clone | (VH) | (VL) |
| Murine | EVQLQQSGPELVKPGASLKISCKASGYTFT | DIVLTQSPAFLAVSLGQRATISCRASESV |
| 02A01 | DFTMHWVKQSHGKSLEWIGLVNPNNGGNIY | DSYDINSMHWYQQKPGQPPKLLIYRASHL |
| | NQNFKGKATLTVDTSSSTAYMELRSLTSED | QSGIPARFSGSGSRTDFTLTINFVEADDV |
| | SSVYYCGRVPLYTSHGMDYWGQGTSVTVSS | ATYYCQQSNEDPLTFGAGTKLELK (SEQ |
| | (SEQ ID NO: 30) | ID NO: 31) |
| | HCDR1-DFTMH (SEQ ID NO: 24) | LCDR1-RASESVDSYDINSMH (SEQ |
| | HCDR2-LVNPNNGGNIYNQNFKG (SEQ | ID NO: 27) |
| | ID NO: 32) | LCDR2-RASHLQS (SEQ ID |
| | HCDR3-VPLYTSHGMDY (SEQ ID | NO: 28) |
| | NO: 26) | LCDR3-QQSNEDPLT (SEQ ID |
| | | NO: 29) |
| Murine | EVQLQQSGPELVKPGASLKISCKASGYTFT | DIVLTQSPAFLAVSLGQRATISCRASESV |
| 02A01 | DFTLHWVKQSHGKSLEWIGLVNPNNGGNIY | DSYDINSMHWYQQKPGQPPKLLIYRASHL |
| VH- | NQNFKGKATLTVDTSSSTAYMELRSLTSED | QSGIPARFSGSGSRTDFTLTINFVEADDV |
| M39L | SSVYYCGRVPLYTSHGMDYWGQGTSVTVSS | ATYYCQQSNEDPLTFGAGTKLELK (SEQ |
| | (SEQ ID NO: 30) | ID NO: 31) |
| | HCDR1-DFTLH (SEQ ID NO: 34) | LCDR1-RASESVDSYDINSMH (SEQ |
| | HCDR2-LVNPNNGGNIYNQNFKG (SEQ | ID NO: 27) |
| | ID NO: 32) | LCDR2-RASHLQS (SEQ ID |
| | HCDR3-VPLYTSHGMDY (SEQ ID | NO: 28) |
| | NO: 26) | LCDR3-QQSNEDPLT (SEQ ID |
| | | NO: 29) |
| 02A01 | HCDR1-DFTXH, wherein X is M | LCDR1-RASESVX₁SYDIX₂SMH, |
| Consensus | or L (SEQ ID NO: 35) | wherein X₁ is D or E; and X₂ |
| 1¹ | HCDR2-LVNPNNGGNIYX₁X₂X₃FX₄G, | is N or Q (SEQ ID NO: 37) |
| | wherein X₁ is N or A; X₂ is Q | LCDR2-X₁ASX₂X₃X₄X₅, wherein |
| | or E; X₃ is N or K; and X₄ is K | X1 is R or D; X₂ is H or N; X₃ |
| | or Q (SEQ ID NO: 36) | is L or R; X₄ is Q or A; and |
| | HCDR3-VPLYTSHGMDY (SEQ ID | X₅ is S or T |
| | NO: 26) | LCDR3-QQSNEDPLT (SEQ ID |
| | | NO: 29) |
| 02A01 | HCDR1-DFTMH (SEQ ID NO: 24) | LCDR1-RASESVX₁SYDIX₂ SMH, |
| Consensus | HCDR2-LVNPNNGGNIYX₁X₂X₃FX₄G, | wherein X₁ is D or E; and X₂ |
| 2² | wherein X₁, X₂, X₃, and X₄ are | is N or Q (SEQ ID NO: 37) |
| | A, E, K, and Q, respectively; | LCDR2-X₁ASX₂X₃X₄X₅, wherein |
| | or X₁, X₂, X₃, and X₄ are N, Q, | X₁, X₂, X₃, X₄, and X₅ are R, |
| | N, and K, respectively (SEQ ID | H, L, Q, and S, respectively; |
| | NO: 38) | or X₁, X₂, X₃, X₄, and X₅ are |
| | HCDR3-VPLYTSHGMDY (SEQ ID | D, N, R, A, and T, |
| | NO: 26) | respectively |
| | | LCDR3-QQSNEDPLT (SEQ ID |
| | | NO: 29) |
| Humanized | EVQLVQSGPEVVKPGATLKISCKASGYTFT | |
| 02A01- | DFTMHWVKQAPGKSLEWIGLVNPNNGGNIY | |
| VH1 | NQNFKGRATLTVDTSTSTAYMELSSLRSED | |
| | TSVYYCGRVPLYTSHGMDYWGQGTLVTVSS | |
| | (SEQ ID NO: 39) | |
| | HCDR1-DFTMH (SEQ ID NO: 24) | |
| | HCDR2-LVNPNNGGNIYNQNFKG (SEQ | |
| | ID NO: 32) | |
| | HCDR3-VPLYTSHGMDY (SEQ ID | |
| | NO: 26) | |
| Humanized | EVQLVQSGAEVKKPGATLKISCKASGYTFT | |
| 02A01- | DFTMHWVQQAPGKGLEWIGLVNPNNGGNIY | |
| VH2 | NQNFKGRATLTVDTSTSTAYMELSSLRSED | |
| | TSVYYCGRVPLYTSHGMDYWGQGTLVTVSS | |
| | (SEQ ID NO: 40) | |
| | HCDR1-DFTMH (SEQ ID NO: 24) | |
| | HCDR2-LVNPNNGGNIYNQNFKG (SEQ | |
| | ID NO: 32) | |
| | HCDR3-VPLYTSHGMDY (SEQ ID | |
| | NO: 26) | |
| Humanized | EVQLVQSGAEVKKPGATLKISCKASGYTFT | |
| 02A01- | DFTMHWVQQAPGKGLEWIGLVNPNNGGNIY | |
| VH3 | AEKFQGRATLTVDTSTSTAYMELSSLRSED | |
| | TSVYYCGRVPLYTSHGMDYWGQGTLVTVSS | |
| | (SEQ ID NO: 41) | |
| | HCDR1-DFTMH (SEQ ID NO: 24) | |
| | HCDR2-LVNPNNGGNIYAEKFQG (SEQ | |
| | ID NO: 25) | |
| | HCDR3-VPLYTSHGMDY (SEQ ID | |
| | NO: 26) | |
| Humanized | EVQLVQSGAEVKKPGATVKISCKASGYTFT | |
| 02A01- | DFTMHWVQQAPGKGLEWIGLVNPNNGGNIY | |
| VH4 | AEKFQGRATLTVDTSTSTAYMELSSLRSED | |
| | TAVYYCGRVPLYTSHGMDYWGQGTLVTVSS | |
| | (SEQ ID NO: 42) | |
| | HCDR1-DFTMH (SEQ ID NO: 24) | |
| | HCDR2-LVNPNNGGNIYAEKFQG (SEQ | |
| | ID NO: 25) | |
| | HCDR3-VPLYTSHGMDY (SEQ ID | |
| | NO: 26) | |
| Humanized | EVQLVQSGAEVKKPGATLKISCKASGYTFT | |
| 02A01- | DYYMHWVQQAPGKGLEWIGLVNPNNGGNIY | |
| VH5 | NQNFKGRATLTVDTSTSTAYMELSSLRSED | |
| | TSVYYCGRVPLYTSHGMDYWGQGTLVTVSS | |
| | (SEQ ID NO: 43) | |
| | HCDR1-DYYMH (SEQ ID NO: 44) | |
| | HCDR2-LVNPNNGGNIYNQNFKG (SEQ | |
| | ID NO: 32) | |
| | HCDR3-VPLYTSHGMDY (SEQ ID | |
| | NO: 26) | |
| Humani | EVQLVQSGAEVKKPGATVKISCKASGYTFT | |
| zed | DFTMHWVQQAPGKGLEWIGLVNPNNGGNIY | |
| 02A01- | AEKFQGRVTLTVDTSTSTAYMELSSLRSED | |
| VH6 | TAVYYCGRVPLYTSHGMDYWGQGTLVTVSS | |
| | (SEQ ID NO: 22) | |
| | HCDR1-DFTMH (SEQ ID NO: 24) | |
| | HCDR2-LVNPNNGGNIYAEKFQG (SEQ | |
| | ID NO: 25) | |
| | HCDR3-VPLYTSHGMDY (SEQ ID | |
| | NO: 26) | |
| Humanized | | EIVLTQSPATLSVSPGERATISCRASESV |
| 02A01- | | DSYDINSMHWYQQKPGQPPRLLIYRASHL |
| VK1 | | QSGIPARFSGSGSRTDFTLTISSVEPEDF |
| | | ATYYCQQSNEDPLTFGQGTKLEIK (SEQ |
| | | ID NO: 45) |
| | | LCDR1-RASESVDSYDINSMH (SEQ |
| | | ID NO: 27) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSVSPGERATISCRASESV |
| 02A01- | | DSYDINSMHWYQQKPGQAPRLLIYRASHL |
| VK2 | | QSGIPARFSGSGSRTDFTLTISSVEPEDF |
| | | ATYYCQQSNEDPLTFGQGTKLEIK (SEQ |
| | | ID NO: 46) |
| | | LCDR1-RASESVDSYDINSMH (SEQ |
| | | ID NO: 27) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSLSPGERATISCRASESV |
| 02A01- | | DSYDINSMHWYQQKPGQAPRLLIYRASHL |
| VK3 | | QSGIPARFSGSGSRTDFTLTISSLEPEDF |
| | | ATYYCQQSNEDPLTFGQGTKLEIK (SEQ |
| | | ID NO: 23) |
| | | LCDR1-RASESVDSYDINSMH (SEQ |
| | | ID NO: 27) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSLSPGERATISCRASESV |
| 02A01- | | ESYDIQSMHWYQQKPGQAPRLLIYRASHL |
| VK3 | | QSGIPARFSGSGSRTDFTLTISSLEPEDF |
| (D30E, N35Q) | | ATYYCQQSNEDPLTFGQGTKLEIK (SEQ |
| | | ID NO: 47) |
| | | LCDR1-RASESVESYDIQSMH (SEQ |
| | | ID NO: 48) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSVSPGERATISCRASESV |
| 02A01-VK4 | | DSYDINSMHWYQQKPGQAPRLLIYDASNR |
| | | ATGIPARFSGSGSRTDFTLTISSVEPEDF |
| | | ATYYCQQSNEDPLTFGQGTKLEIK (SEQ |
| | | ID NO: 49) |
| | | LCDR1-RASESVDSYDINSMH (SEQ |
| | | ID NO: 27) |
| | | LCDR2-DASNRAT (SEQ ID |
| | | NO: 50) |
| | | LCDR3-QQSNEDPLT (SEQ ID |
| | | NO: 29) |

¹02A01 consensus 1 is based on the Kabat CDR sequences of: (a) the heavy chain CDRs of murine 02A01, murine 02A01 VH-M39L, humanized 02A01-VH1, humanized 02A01-VH2, humanized 02A01-VH3, humanized 02A01-VH4, and humanized 02A01-VH6; and (b) the light chain CDRs of murine 02A01, murine 02A01 VH-M39L, humanized 02A01-VK1, humanized 02A01-VK2, humanized 02A01-VK3, humanized 02A01-VK3(D30E/N35Q), and humanized 02A01-VK4.
²02A01 consensus 2 is based on the Kabat CDR sequences of: (a) the heavy chain CDRs of humanized 02A01-VH1, humanized 02A01-VH2, humanized 02A01-VH3, humanized 02A01-VH4, and humanized 02A01-VH6; and (b) the light chain CDRs of humanized 02A01-VK1, humanized 02A01-VK2, humanized 02A01-VK3, humanized 02A01-VK3(D30E/N35Q), and humanized 02A01-VK4.

TABLE 1B
Exemplary silent CD8 antigen-binding sites derived from 02A01 (Chothia)

| | Heavy Chain Variable Domain | Light Chain Variable Domain |
|---|---|---|
| Clone | (VH) | (VL) |
| Murine | EVQLQQSGPELVKPGASLKISCKASGYTFT | DIVLTQSPAFLAVSLGQRATISCRASESV |
| 02A01 | DFTMHWVKQSHGKSLEWIGLVNPNNGGNIY | DSYDINSMHWYQQKPGQPPKLLIYRASHL |
| | NQNFKGKATLTVDTSSSTAYMELRSLTSED | QSGIPARFSGSGSRTDFTLTINFVEADDV |
| | SSVYYCGRVPLYTSHGMDYWGQGTSVTVSS | ATYYCQQSNEDPLTFGAGTKLELK (SEQ |
| | (SEQ ID NO: 30) | ID NO: 31) |
| | HCDR1-GYTFTDF (SEQ ID | LCDR1-RASESVDSYDINSMH (SEQ |
| | NO: 139) | ID NO: 27) |
| | HCDR2-NPNNGG (SEQ ID NO: 140) | LCDR2-RASHLQS (SEQ ID |
| | HCDR3-VPLYTSHGMDY (SEQ ID | NO: 28) |
| | NO: 26) | LCDR3-QQSNEDPLT (SEQ ID |
| | | NO: 29) |
| Murine | EVQLQQSGPELVKPGASLKISCKASGYTFT | DIVLTQSPAFLAVSLGQRATISCRASESV |
| 02A01 | DFTLHWVKQSHGKSLEWIGLVNPNNGGNIY | DSYDINSMHWYQQKPGQPPKLLIYRASHL |
| VH- | NQNFKGKATLTVDTSSSTAYMELRSLTSED | QSGIPARFSGSGSRTDFTLTINFVEADDV |
| M39L | SSVYYCGRVPLYTSHGMDYWGQGTSVTVSS | ATYYCQQSNEDPLTFGAGTKLELK (SEQ |
| | (SEQ ID NO: 33) | ID NO: 31) |
| | HCDR1-GYTFTDF (SEQ ID | LCDR1-RASESVDSYDINSMH (SEQ |
| | NO: 139) | ID NO: 27) |
| | HCDR2-NPNNGG (SEQ ID NO: 140) | LCDR2-RASHLQS (SEQ ID |
| | HCDR3-VPLYTSHGMDY (SEQ ID | NO: 28) |
| | NO: 26) | LCDR3-QQSNEDPLT (SEQ ID |
| | | NO: 29) |
| 02A01 | HCDR1-GYTFTDF (SEQ ID | LCDR1-RASESVX₁SYDIX₂SMH, |
| Consensus | NO: 139) | wherein X₁ is D or E; and X₂ |
| 3³ | HCDR2-NPNNGG (SEQ ID NO: 140) | is N or Q (SEQ ID NO: 37) |
| | HCDR3-VPLYTSHGMDY (SEQ ID | LCDR2-X₁ASX₂X₃X₄X₅, wherein |
| | NO: 26) | X₁ is R or D; X₂ is H or N; X₃ |
| | | is L or R; X₄ is Q or A; and |
| | | X₅ is S or T |
| | | LCDR3-QQSNEDPLT (SEQ ID |
| | | NO: 29) |
| 02A01 | HCDR1-GYTFTDF (SEQ ID | LCDR1-RASESVX₁SYDIX₂ SMH, |
| Consensus | NO: 139) | wherein X₁ is D or E; and X₂ |
| 4⁴ | HCDR2-NPNNGG (SEQ ID NO: 140) | is N or Q (SEQ ID NO: 37) |
| | HCDR3-VPLYTSHGMDY (SEQ ID | LCDR2-X₁ASX₂X₃X₄X₅, wherein |
| | NO: 26) | X₁, X₂, X₃, X₄, and X₅ are R, |
| | | H, L, Q, and S, respectively; |
| | | or X₁, X₂, X₃, X₄, and X₅ are |
| | | D, N, R, A, and T, |
| | | respectively |
| | | LCDR3-QQSNEDPLT (SEQ ID |
| | | NO: 29) |
| Humanized | EVQLVQSGPEVVKPGATLKISCKASGYTFT | |
| 02A01- | DFTMHWVKQAPGKSLEWIGLVNPNNGGNIY | |
| VH1 | NQNFKGRATLTVDTSTSTAYMELSSLRSED | |
| | TSVYYCGRVPLYTSHGMDYWGQGTLVTVSS | |
| | (SEQ ID NO: 39) | |
| | HCDR1-GYTFTDF (SEQ ID | |
| | NO: 139) | |
| | HCDR2-NPNNGG (SEQ ID NO: 140) | |
| | HCDR3-VPLYTSHGMDY (SEQ ID | |
| | NO: 26) | |
| Humanized | EVQLVQSGAEVKKPGATLKISCKASGYTFT | |
| 02A01- | DFTMHWVQQAPGKGLEWIGLVNPNNGGNIY | |
| VH2 | NQNFKGRATLTVDTSTSTAYMELSSLRSED | |
| | TSVYYCGRVPLYTSHGMDYWGQGTLVTVSS | |
| | (SEQ ID NO: 40) | |
| | HCDR1-GYTFTDF (SEQ ID | |
| | NO: 139) | |
| | HCDR2-NPNNGG (SEQ ID NO: 140) | |
| | HCDR3-VPLYTSHGMDY (SEQ ID | |
| | NO: 26) | |
| Humanized | EVQLVQSGAEVKKPGATLKISCKASGYTFT | |
| 02A01- | DFTMHWVQQAPGKGLEWIGLVNPNNGGNIY | |
| VH3 | AEKFQGRATLTVDTSTSTAYMELSSLRSED | |
| | TSVYYCGRVPLYTSHGMDYWGQGTLVTVSS | |
| | (SEQ ID NO: 41) | |
| | HCDR1-GYTFTDF (SEQ ID | |
| | NO: 139) | |
| | HCDR2-NPNNGG (SEQ ID NO: 140) | |
| | HCDR3-VPLYTSHGMDY (SEQ ID | |
| | NO: 26) | |
| Humanized | EVQLVQSGAEVKKPGATVKISCKASGYTFT | |
| 02A01- | DFTMHWVQQAPGKGLEWIGLVNPNNGGNIY | |
| VH4 | AEKFQGRATLTVDTSTSTAYMELSSLRSED | |
| | TAVYYCGRVPLYTSHGMDYWGQGTLVTVSS | |
| | (SEQ ID NO: 42) | |
| | HCDR1-GYTFTDF (SEQ ID | |
| | NO: 139) | |
| | HCDR2-NPNNGG (SEQ ID NO: 140) | |
| | HCDR3-VPLYTSHGMDY (SEQ ID | |
| | NO: 26) | |
| Humanized | EVQLVQSGAEVKKPGATLKISCKASGYTFT | |
| 02A01- | DYYMHWVQQAPGKGLEWIGLVNPNNGGNIY | |
| VH5 | NQNFKGRATLTVDTSTSTAYMELSSLRSED | |
| | TSVYYCGRVPLYTSHGMDYWGQGTLVTVSS | |
| | (SEQ ID NO: 43) | |
| | HCDR1-GYTFTDY (SEQ ID | |
| | NO: 141) | |
| | HCDR2-NPNNGG (SEQ ID NO: 140) | |
| | HCDR3-VPLYTSHGMDY (SEQ ID | |
| | NO: 26) | |
| Humanized | EVQLVQSGAEVKKPGATVKISCKASGYTFT | |
| 02A01- | DFTMHWVQQAPGKGLEWIGLVNPNNGGNIY | |
| VH6 | AEKFQGRVTLTVDTSTSTAYMELSSLRSED | |
| | TAVYYCGRVPLYTSHGMDYWGQGTLVTVSS | |
| | (SEQ ID NO: 22) | |
| | HCDR1-GYTFTDF (SEQ ID | |
| | NO: 139) | |
| | HCDR2-NPNNGG (SEQ ID NO: 140) | |
| | HCDR3-VPLYTSHGMDY (SEQ ID | |
| | NO: 26) | |
| Humanized | | EIVLTQSPATLSVSPGERATISCRASESV |
| 02A01- | | DSYDINSMHWYQQKPGQPPRLLIYRASHL |
| VK1 | | QSGIPARFSGSGSRTDFTLTISSVEPEDF |
| | | ATYYCQQSNEDPLTFGQGTKLEIK (SEQ |
| | | ID NO: 45) |
| | | LCDR1-RASESVDSYDINSMH (SEQ |
| | | ID NO: 27) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSVSPGERATISCRASESV |
| 02A01- | | DSYDINSMHWYQQKPGQAPRLLIYRASHL |
| VK2 | | QSGIPARFSGSGSRTDFTLTISSVEPEDF |
| | | ATYYCQQSNEDPLTFGQGTKLEIK (SEQ |
| | | ID NO: 46) |
| | | LCDR1-RASESVDSYDINSMH (SEQ |
| | | ID NO: 27) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSLSPGERATISCRASESV |
| 02A01- | | DSYDINSMHWYQQKPGQAPRLLIYRASHL |
| VK3 | | QSGIPARFSGSGSRTDFTLTISSLEPEDF |
| | | ATYYCQQSNEDPLTFGQGTKLEIK (SEQ |
| | | ID NO: 23) |
| | | LCDR1-RASESVDSYDINSMH (SEQ |
| | | ID NO: 27) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSLSPGERATISCRASESV |
| 02A01- | | ESYDIQSMHWYQQKPGQAPRLLIYRASHL |
| VK3 | | QSGIPARFSGSGSRTDFTLTISSLEPEDF |
| (D30E, N35Q) | | ATYYCQQSNEDPLTFGQGTKLEIK (SEQ |
| | | ID NO: 47) |
| | | LCDR1-RASESVESYDIQSMH (SEQ |
| | | ID NO: 48) |
| | | LCDR2-RASHLQS (SEQ ID |
| | | NO: 28) |
| | | LCDR3-QQSNEDPLT (SEQ ID |
| | | NO: 29) |
| Humanized | | EIVLTQSPATLSVSPGERATISCRASESV |
| 02A01- | | DSYDINSMHWYQQKPGQAPRLLIYDASNR |
| VK4 | | ATGIPARFSGSGSRTDFTLTISSVEPEDF |
| | | ATYYCQQSNEDPLTFGQGTKLEIK (SEQ |
| | | ID NO: 49) |
| | | LCDR1-RASESVDSYDINSMH (SEQ |
| | | ID NO: 27) |
| | | LCDR2-DASNRAT (SEQ ID |
| | | NO: 50) |
| | | LCDR3-QQSNEDPLT (SEQ ID |
| | | NO: 29) |

³02A01 consensus 3 is based on the Chothia CDR sequences of: (a) the heavy chain CDRs of murine 02A01, murine 02A01 VH-M39L, humanized 02A01-VH1, humanized 02A01-VH2, humanized 02A01-VH3, humanized 02A01-VH4, and humanized 02A01-VH6; and (b) the light chain CDRs of murine 02A01, murine 02A01 VH-M39L, humanized 02A01-VK1, humanized 02A01-VK2, humanized 02A01-VK3, humanized 02A01-VK3(D30E/N35Q), and humanized 02A01-VK4.
⁴02A01 consensus 4 is based on the Chothia CDR sequences of: (a) the heavy chain CDRs of humanized 02A01-VH1, humanized 02A01-VH2, humanized 02A01-VH3, humanized 02A01-VH4, and humanized 02A01-VH6; and (b) the light chain CDRs of humanized 02A01-VK1, humanized 02A01-VK2, humanized 02A01-VK3, humanized 02A01-VK3(D30E/N35Q), and humanized 02A01-VK4.

TABLE 2A
Exemplary CD8 antigen-binding sites derived from 03A01 (Kabat)

| | Heavy Chain Variable Domain | Light Chain Variable Domain |
|---|---|---|
| Clone | (VH) | (VL) |
| 03A01 | DVQFQESGPGLVKPSQSLSLTCTVTGYSIT | DIQMTQSSSYLSVSLGGRVTITCKASDHI |
| | SDFAWNWIRQFPGNELEWMGYISYSGDTNY | NNWLAWYQQKPGNAPRLLISGATSLETGV |
| | NPSLRSRISITRDTSKNQFFLQLDSVTTED | PSRFTGSGSGLDYTLSITSLQTEDVATYY |
| | TATYYCTIRGYYTDSAFVFWGQGTLVTVSA | CQQYWNSPFTFGSGTKLEIK (SEQ ID |
| | (SEQ ID NO: 51) | NO: 52) |
| | HCDR1-SDFAWN (SEQ ID NO: 53) | LCDR1-KASDHINNWLA (SEQ ID |
| | HCDR2-YISYSGDTNYNPSLRS (SEQ | NO: 56) |
| | ID NO: 54) | LCDR2-GATSLET (SEQ ID |
| | HCDR3-RGYYTDSAFVF (SEQ ID | NO: 57) |
| | NO: 55) | LCDR3-QQYWNSPFT (SEQ ID |
| | | NO: 58) |
| 03A01 | DVQFQESGPGLVKPSQSLSLTCTVTGYSIT | DIQMTQSSSYLSVSLGGRVTITCKASDHI |
| VH- | SDFAWNWIRQFPGNELEWMGYISYSGETNY | NNWLAWYQQKPGNAPRLLISGATSLETGV |
| D64E | NPSLRSRISITRDTSKNQFFLQLDSVTTED | PSRFTGSGSGLDYTLSITSLQTEDVATYY |
| | TATYYCTIRGYYTDSAFVFWGQGTLVTVSA | CQQYWNSPFTFGSGTKLEIK (SEQ ID |
| | (SEQ ID NO: 59) | NO: 52) |
| | HCDR1-SDFAWN (SEQ ID NO: 53) | LCDR1-KASDHINNWLA (SEQ ID |
| | HCDR2-YISYSGETNYNPSLRS (SEQ | NO: 56) |
| | ID NO 60) | LCDR2-GATSLET (SEQ ID |
| | HCDR3-RGYYTDSAFVF (SEQ ID | NO: 57) |
| | NO: 55) | LCDR3-QQYWNSPFT (SEQ ID |
| | | NO: 58) |
| 03A01 | HCDR1-SDFAWN (SEQ ID NO: 53) | LCDR1-KASDHINNWLA (SEQ ID |
| Consensus | HCDR2-YISYSGX₁TX₂YNPSLX₃S, | NO: 56) |
| 1⁵ | wherein X₁ is D or E; X₂ is N | LCDR2-GATSLET (SEQ ID |
| | or Y; and X₃ is R or K (SEQ ID | NO: 57) |
| | NO: 61) | LCDR3-QQYWNSPFT (SEQ ID |
| | HCDR3-RGYYTDSAFVF (SEQ ID | NO: 58) |
| | NO: 55) | |
| 03A01 | HCDR1-SDFAWN (SEQ ID NO: 53) | LCDR1-KASDHINNWLA (SEQ ID |
| Consensus | HCDR2-YISYSGDTX₁YNPSLX₂S, | NO: 56) |
| 2⁶ | wherein X₁ and X₂ are N and R, | LCDR2-GATSLET (SEQ ID |
| | respectively; X₁ and X₂ are N | NO: 57) |
| | and K, respectively, or X₁ and | LCDR3-QQYWNSPFT (SEQ ID |
| | X₂ are Y and K, respectively | NO: 58) |
| | (SEQ ID NO: 62) | |
| | HCDR3-RGYYTDSAFVF (SEQ ID | |
| | NO: 55) | |
| Humanized | EVQFQESGPGLVKPSQTLSLTCTVTGYSIT | |
| 03A01- | SDFAWNWIRQPPGKELEWMGYISYSGDTNY | |
| VH1 | NPSLRSRITISRDTSKNQFSLKLSSVTAAD | |
| | TATYYCTIRGYYTDSAFVFWGQGTLVTVSS | |
| | (SEQ ID NO: 63) | |
| | HCDR1-SDFAWN (SEQ ID NO: 53) | |
| | HCDR2-YISYSGDTNYNPSLRS (SEQ | |
| | ID NO: 54) | |
| | HCDR3-RGYYTDSAFVF (SEQ ID | |
| | NO: 55) | |
| Humanized | EVQFQESGPGLVKPSQTLSLTCTVSGYSIT | |
| 03A01- | SDFAWNWIRQPPGKGLEWMGYISYSGDTNY | |
| VH2 | NPSLRSRITISRDTSKNQFSLKLSSVTAAD | |
| | TAVYYCTIRGYYTDSAFVFWGQGTLVTVSS | |
| | (SEQ ID NO: 64) | |
| | HCDR1-SDFAWN (SEQ ID NO: 53) | |
| | HCDR2-YISYSGDTNYNPSLRS (SEQ | |
| | ID NO: 54) | |
| | HCDR3-RGYYTDSAFVF (SEQ ID | |
| | NO: 55) | |
| Humanized | EVQFQESGPGLVKPSQTLSLTCTVSGYSIT | |
| 03A01- | SDFAWNWIRQPPGKGLEWMGYISYSGDTNY | |
| VH3 | NPSLKSRVTISRDTSKNQFSLKLSSVTAAD | |
| | TAVYYCTIRGYYTDSAFVFWGQGTLVTVSS | |
| | (SEQ ID NO: 65) | |
| | HCDR1-SDFAWN (SEQ ID NO: 53) | |
| | HCDR2-YISYSGDTNYNPSLKS (SEQ | |
| | ID NO: 66) | |
| | HCDR3-RGYYTDSAFVF (SEQ ID | |
| | NO: 55) | |
| Humanized | EVQFQESGPGLVKPSQTLSLTCTVSGYSIT | |
| 03A01- | SDFAWNWIRQPPGKGLEWIGYISYSGDTYY | |
| VH4 | NPSLKSRVTISRDTSKNQFSLKLSSVTAAD | |
| | TAVYYCTIRGYYTDSAFVFWGQGTLVTVSS | |
| | (SEQ ID NO: 67) | |
| | HCDR1-SDFAWN (SEQ ID NO: 53) | |
| | HCDR2-YISYSGDTYYNPSLKS (SEQ | |
| | ID NO: 68) | |
| | HCDR3-RGYYTDSAFVF (SEQ ID | |
| | NO: 55) | |
| Humanized | | DIQMTQSSSTLSVSLGDRVTITCKASDHI |
| 03A01- | | NNWLAWYQQKPGKAPRLLISGATSLETGV |
| VK1 | | PSRFSGSGSGLEYTLTISSLQPDDFATYY |
| | | CQQYWNSPFTFGQGTKLEIK (SEQ ID |
| | | NO: 69) |
| | | LCDR1-KASDHINNWLA (SEQ ID |
| | | NO: 56) |
| | | LCDR2-GATSLET (SEQ ID |
| | | NO: 57) |
| | | LCDR3-QQYWNSPFT (SEQ ID |
| | | NO: 58) |
| Humanized | | DIQMTQSPSTLSVSVGDRVTITCKASDHI |
| 03A01- | | NNWLAWYQQKPGKAPKLLISGATSLETGV |
| VK2 | | PSRFSGSGSGLEYTLTISSLQPDDFATYY |
| | | CQQYWNSPFTFGQGTKLEIK (SEQ ID |
| | | NO: 70) |
| | | LCDR1-KASDHINNWLA (SEQ ID |
| | | NO: 56) |
| | | LCDR2-GATSLET (SEQ ID |
| | | NO: 57) |
| | | LCDR3-QQYWNSPFT (SEQ ID |
| | | NO: 58) |
| Humanized | | DIQMTQSPSTLSASVGDRVTITCKASDHI |
| 03A01- | | NNWLAWYQQKPGKAPKLLISGATSLETGV |
| VK3 | | PSRFSGSGSGLEYTLTISSLQPDDFATYY |
| | | CQQYWNSPFTFGQGTKLEIK (SEQ ID |
| | | NO: 71) |
| | | LCDR1-KASDHINNWLA (SEQ ID |
| | | NO: 56) |
| | | LCDR2-GATSLET (SEQ ID |
| | | NO: 57) |
| | | LCDR3-QQYWNSPFT (SEQ ID |
| | | NO: 58) |
| Humanized | | DIQMTQSPSTLSVSVGDRVTITCRASQSI |
| 03A01- | | SSWLAWYQQKPGKAPKLLISGATSLETGV |
| VK4 | | PSRFSGSGSGLEYTLTISSLQPDDFATYY |
| | | CQQYWNSPFTFGQGTKLEIK (SEQ ID |
| | | NO: 72) |
| | | LCDR1-RASQSISSWLA (SEQ ID |
| | | NO: 73) |
| | | LCDR2-GATSLET (SEQ ID |
| | | NO: 57) |
| | | LCDR3-QQYWNSPFT (SEQ ID |
| | | NO: 58) |

⁵03A01 consensus 1 is based on the Kabat CDR sequences of: (a) the heavy chain CDRs of murine 03A01, murine 03A01 VH-D64E, humanized 03A01-VH1, humanized 03A01-VH2, humanized 03A01-VH3, and humanized 03A01-VH4; and (b) the light chain CDRs of murine 03A01, murine 03A01 VH-D64E, humanized 03A01-VK1, humanized 03A01-VK2, and humanized 03A01-VK3.
⁶03A01 consensus 2 is based on the Kabat CDR sequences of: (a) the heavy chain CDRs of humanized 03A01-VH1, humanized 03A01-VH2, humanized 03A01-VH3, and humanized 03A01-VH4; and (b) the light chain CDRs of humanized 03A01-VK1, humanized 03A01-VK2, and humanized 03A01-VK3.

TABLE 2B
Exemplary silent CD8 antigen-binding sites derived from 03A01 (Chothia)

| | Heavy Chain Variable Domain | Light Chain Variable Domain |
|---|---|---|
| Clone | (VH) | (VL) |
| 03A01 | DVQFQESGPGLVKPSQSLSLTCTVTGYSIT | DIQMTQSSSYLSVSLGGRVTITCKASDHI |
| | SDFAWNWIRQFPGNELEWMGYISYSGDTNY | NNWLAWYQQKPGNAPRLLISGATSLETGV |
| | NPSLRSRISITRDTSKNQFFLQLDSVTTED | PSRFTGSGSGLDYTLSITSLQTEDVATYY |
| | TATYYCTIRGYYTDSAFVFWGQGTLVTVSA | CQQYWNSPFTFGSGTKLEIK (SEQ ID |
| | (SEQ ID NO: 51) | NO: 52) |
| | HCDR1-GYSITSDF (SEQ ID | LCDR1-KASDHINNWLA (SEQ ID |
| | NO: 142) | NO: 56) |
| | HCDR2-SYSGD (SEQ ID NO: 143) | LCDR2-GATSLET (SEQ ID |
| | HCDR3-RGYYTDSAFVF (SEQ ID | NO: 57) |
| | NO: 55) | LCDR3-QQYWNSPFT (SEQ ID |
| | | NO: 58) |
| 03A01 | DVQFQESGPGLVKPSQSLSLTCTVTGYSIT | DIQMTQSSSYLSVSLGGRVTITCKASDHI |
| VH- | SDFAWNWIRQFPGNELEWMGYISYSGETNY | NNWLAWYQQKPGNAPRLLISGATSLETGV |
| D64E | NPSLRSRISITRDTSKNQFFLQLDSVTTED | PSRFTGSGSGLDYTLSITSLQTEDVATYY |
| | TATYYCTIRGYYTDSAFVFWGQGTLVTVSA | CQQYWNSPFTFGSGTKLEIK (SEQ ID |
| | (SEQ ID NO: 59) | NO: 52) |
| | HCDR1-GYSITSDF (SEQ ID | LCDR1-KASDHINNWLA (SEQ ID |
| | NO: 142) | NO: 56) |
| | HCDR2-SYSGE (SEQ ID NO: 144) | LCDR2-GATSLET (SEQ ID |
| | HCDR3-RGYYTDSAFVF (SEQ ID | NO: 57) |
| | NO: 55) | LCDR3-QQYWNSPFT (SEQ ID |
| | | NO: 58) |
| 03A01 | HCDR1-GYSITSDF (SEQ ID | LCDR1-KASDHINNWLA (SEQ ID |
| Consensus | NO: 142) | NO: 56) |
| 3⁷ | HCDR2-SYSGX₁, wherein X₁ is D | LCDR2-GATSLET (SEQ ID |
| | or E; (SEQ ID NO: 145) | NO: 57) |
| | HCDR3-RGYYTDSAFVF (SEQ ID | LCDR3-QQYWNSPFT (SEQ ID |
| | NO: 55) | NO: 58) |
| Humanized | EVQFQESGPGLVKPSQTLSLTCTVTGYSIT | |
| 03A01- | SDFAWNWIRQPPGKELEWMGYISYSGDTNY | |
| VH1 | NPSLRSRITISRDTSKNQFSLKLSSVTAAD | |
| | TATYYCTIRGYYTDSAFVFWGQGTLVTVSS | |
| | (SEQ ID NO: 63) | |
| | HCDR1-GYSITSDF (SEQ ID | |
| | NO: 142) | |
| | HCDR2-SYSGD (SEQ ID NO: 143) | |
| | HCDR3-RGYYTDSAFVF (SEQ ID | |
| | NO: 55) | |
| Humanized | EVQFQESGPGLVKPSQTLSLTCTVSGYSIT | |
| 03A01- | SDFAWNWIRQPPGKGLEWMGYISYSGDTNY | |
| VH2 | NPSLRSRITISRDTSKNQFSLKLSSVTAAD | |
| | TAVYYCTIRGYYTDSAFVFWGQGTLVTVSS | |
| | (SEQ ID NO: 64) | |
| | HCDR1-GYSITSDF (SEQ ID | |
| | NO: 142) | |
| | HCDR2-SYSGD (SEQ ID NO: 143) | |
| | HCDR3-RGYYTDSAFVF (SEQ ID | |
| | NO: 55) | |
| Humanized | EVQFQESGPGLVKPSQTLSLTCTVSGYSIT | |
| 03A01- | SDFAWNWIRQPPGKGLEWMGYISYSGDTNY | |
| VH3 | NPSLKSRVTISRDTSKNQFSLKLSSVTAAD | |
| | TAVYYCTIRGYYTDSAFVFWGQGTLVTVSS | |
| | (SEQ ID NO: 65) | |
| | HCDR1-GYSITSDF (SEQ ID | |
| | NO:142) | |
| | HCDR2 SYSGD (SEQ ID NO: 143) | |
| | HCDR3-RGYYTDSAFVF (SEQ ID | |
| | NO: 55) | |
| Humanized | EVQFQESGPGLVKPSQTLSLTCTVSGYSIT | |
| 03A01- | SDFAWNWIRQPPGKGLEWIGYISYSGDTYY | |
| VH4 | NPSLKSRVTISRDTSKNQFSLKLSSVTAAD | |
| | TAVYYCTIRGYYTDSAFVFWGQGTLVTVSS | |
| | (SEQ ID NO: 67) | |
| | HCDR1-GYSITSDF (SEQ ID | |
| | NO: 142) | |
| | HCDR2-SYSGD (SEQ ID NO: 143) | |
| | HCDR3-RGYYTDSAFVF (SEQ ID | |
| | NO: 55) | |
| Humanized | | DIQMTQSSSTLSVSLGDRVTITCKASDHI |
| 03A01- | | NNWLAWYQQKPGKAPRLLISGATSLETGV |
| VK1 | | PSRFSGSGSGLEYTLTISSLQPDDFATYY |
| | | CQQYWNSPFTFGQGTKLEIK (SEQ ID |
| | | NO: 69) |
| | | LCDR1-KASDHINNWLA (SEQ ID |
| | | NO: 56) |
| | | LCDR2-GATSLET (SEQ ID |
| | | NO: 57) |
| | | LCDR3-QQYWNSPFT (SEQ ID |
| | | NO: 58) |
| Humanized | | DIQMTQSPSTLSVSVGDRVTITCKASDHI |
| 03A01- | | NNWLAWYQQKPGKAPKLLISGATSLETGV |
| VK2 | | PSRFSGSGSGLEYTLTISSLQPDDFATYY |
| | | CQQYWNSPFTFGQGTKLEIK (SEQ ID |
| | | NO: 70) |
| | | LCDR1-KASDHINNWLA (SEQ ID |
| | | NO: 56) |
| | | LCDR2-GATSLET (SEQ ID |
| | | NO: 57) |
| | | LCDR3-QQYWNSPFT (SEQ ID |
| | | NO: 58) |
| Humanized | | DIQMTQSPSTLSASVGDRVTITCKASDHI |
| 03A01- | | NNWLAWYQQKPGKAPKLLISGATSLETGV |
| VK3 | | PSRFSGSGSGLEYTLTISSLQPDDFATYY |
| | | CQQYWNSPFTFGQGTKLEIK (SEQ ID |
| | | NO: 71) |
| | | LCDR1-KASDHINNWLA (SEQ ID |
| | | NO: 56) |
| | | LCDR2-GATSLET (SEQ ID |
| | | NO: 57) |
| | | LCDR3-QQYWNSPFT (SEQ ID |
| | | NO: 58) |
| Humanized | | DIQMTQSPSTLSVSVGDRVTITCRASQSI |
| 03A01- | | SSWLAWYQQKPGKAPKLLISGATSLETGV |
| VK4 | | PSRFSGSGSGLEYTLTISSLQPDDFATYY |
| | | CQQYWNSPFTFGQGTKLEIK (SEQ ID |
| | | NO:72) |
| | | LCDR1-RASQSISSWLA (SEQ ID |
| | | NO: 73) |
| | | LCDR2-GATSLET (SEQ ID |
| | | NO: 57) |
| | | LCDR3-QQYWNSPFT (SEQ ID |
| | | NO: 58) |

⁷03A01 consensus 3 is based on the Chothia CDR sequences of: (a) the heavy chain CDRs of murine 03A01, murine 03A01 VH-D64E, humanized 03A01-VH1, humanized 03A01-VH2, humanized 03A01-VH3, and humanized 03A01-VH4; and (b) the light chain CDRs of murine 03A01, murine 03A01 VH-D64E, humanized 03A01-VK1, humanized 03A01-VK2, and humanized 03A01-VK3.

TABLE 3A
Additional exemplary CD8 antigen-binding sites (Kabat)

| | Heavy Chain Variable Domain | Light Chain Variable Domain |
|---|---|---|
| Clone | (VH) | (VL) |
| 05F10 | EVQLVESGGGLVKPGGSLKLSCAASGFTFS | DVVMTQTPLSLPVSLEDQASISCRSSQSL |
| | SFAMSWVRQTPAKRLEWVATISSGDLSANY | EHSNGNTYLHWYLQKPGQSPRLLIYKVSN |
| | PDTVKGRFTISRDNAKNTLYLQMSSLRSED | RFSGVPDRFSGSGSGTDFTLKISRVEAED |
| | TAMYYCTRPPFYFSNDWFAYWGQGTLVTVS | LGVYFCSQSTHVPFTFGSGTKLEIK |
| | A (SEQ ID NO: 74) | (SEQ ID NO: 75) |
| | HCDR1-SFAMS (SEQ ID NO: 76) | LCDR1-RSSQSLEHSNGNTYLH (SEQ |
| | HCDR2-TISSGDLSANYPDTVKG (SEQ | ID NO: 79) |
| | ID NO: 77) | LCDR2-KVSNRFS (SEQ ID |
| | HCDR3-PPFYFSNDWFAY (SEQ ID | NO: 80) |
| | NO: 78) | LCDR3-SQSTHVPFT (SEQ ID |
| | | NO: 81) |
| 11F05 | DVQLQESGPGLVKPSQSLSLTCTVTGYSIT | DIQMTQSSSYLSVSLGGRVTITCKASDHI |
| | SDYAWNWIRQFPGNKLEWMGYISYSGSTSY | NNWLAWYQQKPGNAPRLLISDATSLETGV |
| | NPSLKSRISITRDTSKNQFFLHLNSVTTED | PSRFSGSVSGMDYTLSITSLQTEDVATYY |
| | TATYYCARRGYYSHSAFTYWGQGTLVTVSA | CQQYWSTPFTFGSGTKLEIE (SEQ ID |
| | (SEQ ID NO: 82) | NO: 83) |
| | HCDR1-SDYAWN (SEQ ID NO: 84) | LCDR1-KASDHINNWLA (SEQ ID |
| | HCDR2-YISYSGSTSYNPSLKS (SEQ | NO: 56) |
| | ID NO: 85) | LCDR2-DATSLET (SEQ ID |
| | HCDR3-RGYYSHSAFTY (SEQ ID | NO: 87) |
| | NO: 86) | LCDR3-QQYWSTPFT (SEQ ID |
| | | NO: 88) |
| 04D08 | EVQLQQSGPELEKPGASVKISCKASGYSFT | DIVMTQSPSSLTVTAGEKVTMSCKSSQSL |
| | GYSMNWVKQSNGKSLEWIGNIDPYYGGTSY | LNIGNQNNYLTWYQQKPGQPPKLLIYWAS |
| | NQNFKGKATLTVDKSSSTAYMQLKSLTSED | TRESGVPDRFTGSGSGTDFTLTISSVQAE |
| | SAVYYCARSYFGNYEDTWFAYWGQGTLVTV | DLAVYYCQNDYSYPLTFGAGTKLELK |
| | SA (SEQ ID NO: 89) | (SEQ ID NO: 90) |
| | HCDR1-GYSMN (SEQ ID NO: 91) | LCDR1-KSSQSLLNIGNQNNYLT |
| | HCDR2-NIDPYYGGTSYNQNFKG (SEQ | (SEQ ID NO: 94) |
| | ID NO: 92) | LCDR2-WASTRES (SEQ ID |
| | HCDR3-SYFGNYEDTWFAY (SEQ ID | NO: 95) |
| | NO: 93) | LCDR3-QNDYSYPLT (SEQ ID |
| | | NO: 96) |

TABLE 3B
Additional exemplary silent CD8 antigen-binding sites (Chothia)

| | Heavy Chain Variable Domain | Light Chain Variable Domain |
|---|---|---|
| Clone | (VH) | (VL) |
| 05F10 | EVQLVESGGGLVKPGGSLKLSCAASGFTF | DVVMTQTPLSLPVSLEDQASISCRSSQSL |
| | SSFAMSWVRQTPAKRLEWVATISSGDLSA | EHSNGNTYLHWYLQKPGQSPRLLIYKVSN |
| | NYPDTVKGRFTISRDNAKNTLYLQMSSLR | RFSGVPDRFSGSGSGTDFTLKISRVEAED |
| | SEDTAMYYCTRPPFYFSNDWFAYWGQGTL | LGVYFCSQSTHVPFTFGSGTKLEIK |
| | VTVSA (SEQ ID NO: 74) | (SEQ ID NO: 75) |
| | HCDR1-GFTFSSF (SEQ ID | LCDR1-RSSQSLEHSNGNTYLH (SEQ |
| | NO: 146) | ID NO: 79) |
| | HCDR2-SSGDLS (SEQ ID | LCDR2-KVSNRFS (SEQ ID |
| | NO: 147) | NO: 80) |
| | HCDR3-PPFYFSNDWFAY (SEQ ID | LCDR3-SQSTHVPFT (SEQ ID |
| | NO: 78) | NO: 81) |
| 11F05 | DVQLQESGPGLVKPSQSLSLTCTVTGYSI | DIQMTQSSSYLSVSLGGRVTITCKASDHI |
| | TSDYAWNWIRQFPGNKLEWMGYISYSGST | NNWLAWYQQKPGNAPRLLISDATSLETGV |
| | SYNPSLKSRISITRDTSKNQFFLHLNSVT | PSRFSGSVSGMDYTLSITSLQTEDVATYY |
| | TEDTATYYCARRGYYSHSAFTYWGQGTLV | CQQYWSTPFTFGSGTKLEIE (SEQ ID |
| | TVSA (SEQ ID NO: 82) | NO: 83) |
| | HCDR1-GYSITSDY (SEQ ID | LCDR1-KASDHINNWLA (SEQ ID |
| | NO: 148) | NO: 56) |
| | HCDR2-SYSGS (SEQ ID NO: 149) | LCDR2-DATSLET (SEQ ID |
| | HCDR3-RGYYSHSAFTY (SEQ ID | NO: 87) |
| | NO: 86) | LCDR3-QQYWSTPFT (SEQ ID |
| | | NO: 88) |
| 04D08 | EVQLQQSGPELEKPGASVKISCKASGYSF | DIVMTQSPSSLTVTAGEKVTMSCKSSQSL |
| | TGYSMNWVKQSNGKSLEWIGNIDPYYGGT | LNIGNQNNYLTWYQQKPGQPPKLLIYWAS |
| | SYNQNFKGKATLTVDKSSSTAYMQLKSLT | TRESGVPDRFTGSGSGTDFTLTISSVQAE |
| | SEDSAVYYCARSYFGNYEDTWFAYWGQGT | DLAVYYCQNDYSYPLTFGAGTKLELK |
| | LVTVSA (SEQ ID NO: 89) | (SEQ ID NO: 90) |
| | HCDR1-GYSFTGY (SEQ ID | LCDR1-KSSQSLLNIGNQNNYLT |
| | NO: 150) | (SEQ ID NO: 94) |
| | HCDR2-DPYYGG (SEQ ID | LCDR2-WASTRES (SEQ ID |
| | NO: 151) | NO: 95) |
| | HCDR3-SYFGNYEDTWFAY (SEQ ID | LCDR3-QNDYSYPLT (SEQ ID |
| | NO: 93) | NO: 96) |

In some aspects, the antigen-binding site that binds CD8 comprises a heavy chain and a light chain. In some aspects, the antigen-binding site that binds CD8 comprises two heavy chains and two light chains. In some aspects, the heavy chain comprises the amino acid sequence set forth in SEQ ID NO: 1. In some aspects, the heavy chain comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 1.

In some aspects, the light chain comprises the amino acid sequence set forth in SEQ ID NO: 2. In some aspects, the light chain comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 2.

In some aspects, the heavy chain comprises the amino acid sequence set forth in SEQ ID NO: 3. In some aspects, the heavy chain comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 3.

In some aspects, the light chain comprises the amino acid sequence set forth in SEQ ID NO: 4. In some aspects, the light chain comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 4.

In some aspects, the heavy chain comprises the amino acid sequence set forth in SEQ ID NO: 5. In some aspects, the heavy chain comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 5.

In some aspects, the light chain comprises the amino acid sequence set forth in SEQ ID NO: 6. In some aspects, the light chain comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 6.

In some aspects, the antigen-binging site that binds CD8 comprises a heavy chain variable domain (VH) and a light chain variable domain (VL). In some aspects, the VH comprises the amino acid sequence set forth in SEQ ID NO: 22. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 22.

In some aspects, the VH comprises the amino acid sequence set forth in SEQ ID NO: 30. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 30.

In some aspects, the VH comprises the amino acid sequence set forth in SEQ ID NO: 33. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 33.

In some aspects, the VH comprises the amino acid sequence set forth in SEQ ID NO: 39. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 39.

In some aspects, the VH comprises the amino acid sequence set forth in SEQ ID NO: 40. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 40.

In some aspects, the VH comprises the amino acid sequence set forth in SEQ ID NO: 41. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 41.

In some aspects, the VH comprises the amino acid sequence set forth in SEQ ID NO: 42. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 42.

In some aspects, the VH comprises the amino acid sequence set forth in SEQ ID NO: 43. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 43.

In some aspects, the VH comprises the amino acid sequence set forth in SEQ ID NO: 51. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 51.

In some aspects, the VH comprises the amino acid sequence set forth in SEQ ID NO: 59. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 59.

In some aspects, the VH comprises the amino acid sequence set forth in SEQ ID NO: 63. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 63.

In some aspects, the VH comprises the amino acid sequence set forth in SEQ ID NO: 64. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 64.

In some aspects, the VH comprises the amino acid sequence set forth in SEQ ID NO: 65. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 65.

In some aspects, the VH comprises the amino acid sequence set forth in SEQ ID NO: 67. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 67.

In some aspects, the VH comprises the amino acid sequence set forth in SEQ ID NO: 74. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 74.

In some aspects, the VH comprises the amino acid sequence set forth in SEQ ID NO: 82. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 82.

In some aspects, the VH comprises the amino acid sequence set forth in SEQ ID NO: 89. In some aspects, the VH comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 89.

In some aspects, the VL comprises the amino acid sequence set forth in SEQ ID NO: 23. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 23.

In some aspects, the VL comprises the amino acid sequence set forth in SEQ ID NO: 31. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 31.

In some aspects, the VL comprises the amino acid sequence set forth in SEQ ID NO: 45. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 45.

In some aspects, the VL comprises the amino acid sequence set forth in SEQ ID NO: 46. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 46.

In some aspects, the VL comprises the amino acid sequence set forth in SEQ ID NO: 47. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 47.

In some aspects, the VL comprises the amino acid sequence set forth in SEQ ID NO: 49. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 49.

In some aspects, the VL comprises the amino acid sequence set forth in SEQ ID NO: 52. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 52.

In some aspects, the VL comprises the amino acid sequence set forth in SEQ ID NO: 69. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 69.

In some aspects, the VL comprises the amino acid sequence set forth in SEQ ID NO: 70. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 70.

In some aspects, the VL comprises the amino acid sequence set forth in SEQ ID NO: 71. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 71.

In some aspects, the VL comprises the amino acid sequence set forth in SEQ ID NO: 72. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 72.

In some aspects, the VL comprises the amino acid sequence set forth in SEQ ID NO: 75. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 75.

In some aspects, the VL comprises the amino acid sequence set forth in SEQ ID NO: 83. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 83.

In some aspects, the VL comprises the amino acid sequence set forth in SEQ ID NO: 90. In some aspects, the VL comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 90.

In some aspects, the VH and the VL comprise amino acid sequences having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NOs: 22 and 47; 39 and 47; 40 and 47; 41 and 47; or 42 and 47, respectively.

In some aspects, the VH and the VL comprise amino acid sequences having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NOs: 39 and 45; 40 and 45; 41 and 45; 42 and 45; 22 and 45; 39 and 46; 40 and 46; 41 and 46; 42 and 46; 22 and 46; 39 and 23; 40 and 23; 41 and 23; 42 and 23; or 22 and 23, respectively.

In some aspects, the VH and the VL comprise amino acid sequences having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NOs: 30 and 31, respectively, or 33 and 31, respectively.

In some aspects, the VH and the VL comprise amino acid sequences having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NOs: 39 and 49; 40 and 49; 41 and 49; 42 and 49; or 22 and 49, respectively.

In some aspects, the heavy chain comprises complementarity determining regions HCDR1, HCDR2, and HCDR3. In some aspects, the VH comprises complementarity determining regions HCDR1, HCDR2, and HCDR3.

In some aspects, the HCDR1 comprises the amino acid sequence set forth in SEQ ID NOs: 24, 34, 35, 44, 53, 76, 84, 91, 139, 141, 142, 146, 148, or 150. In some aspects, the HCDR2 comprises the amino acid sequence set forth in SEQ ID NOs: 25, 32, 36, 38, 54, 60, 61, 62, 66, 68, 77, 85, 92, 140, 143, 144, 145, 147, 149, or 151. In some aspects, the HCDR3 comprises the amino acid sequence set forth in SEQ ID NOs: 26, 55, 78, 86, or 93.

In some aspects, the HCDR1, HCDR2, and HCDR3 comprise the amino acid sequences set forth in SEQ ID NOs: 24, 32, and 26, respectively. In some aspects, the HCDR1, HCDR2, and HCDR3 comprise the amino acid sequences set forth in SEQ ID NOs: 34, 32, and 26, respectively. In some aspects, the HCDR1, HCDR2, and HCDR3 comprise the amino acid sequences set forth in SEQ ID NOs: 24, 25, and 26, respectively. In some aspects, the HCDR1, HCDR2, and HCDR3 comprise the amino acid sequences set forth in SEQ ID NOs: 44, 32, and 26, respectively. In some aspects, the HCDR1, HCDR2, and HCDR3 comprise the amino acid sequences set forth in SEQ ID NOs: 53, 54, and 55, respectively. In some aspects, the HCDR1, HCDR2, and HCDR3 comprise the amino acid sequences set forth in SEQ ID NOs: 53, 60, and 55, respectively. In some aspects, the HCDR1, HCDR2, and HCDR3 comprise the amino acid sequences set forth in SEQ ID NOs: 35, 36, and 26, respectively. In some aspects, the HCDR1, HCDR2, and HCDR3 comprise the amino acid sequences set forth in SEQ ID NOs: 24, 38, and 26, respectively. In some aspects, the HCDR1, HCDR2, and HCDR3 comprise the amino acid sequences set forth in SEQ ID NOs: 53, 61, and 55, respectively. In some aspects, the HCDR1, HCDR2, and HCDR3 comprise the amino acid sequences set forth in SEQ ID NOs: 53, 62, and 55, respectively. In some aspects, the HCDR1, HCDR2, and HCDR3 comprise the amino acid sequences set forth in SEQ ID NOs: 53, 66, and 55, respectively. In some aspects, the HCDR1, HCDR2, and HCDR3 comprise the amino acid sequences set forth in SEQ ID NOs: 53, 68, and 55, respectively. In some aspects, the HCDR1, HCDR2, and HCDR3 comprise the amino acid sequences set forth in SEQ ID NOs: 76, 77, and 78, respectively. In some aspects, the HCDR1, HCDR2, and HCDR3 comprise the amino acid sequences set forth in SEQ ID NOs: 84, 85, and 86, respectively. In some aspects, the HCDR1, HCDR2, and HCDR3 comprise the amino acid sequences set forth in SEQ ID NOs: 91, 92, and 93, respectively. In some aspects, the HCDR1, HCDR2, and HCDR3 comprise the amino acid sequences set forth in SEQ ID NOs: 139, 140, and 26, respectively. In some aspects, the HCDR1, HCDR2, and HCDR3 comprise the amino acid sequences set forth in SEQ ID NOs: 141, 140, and 26, respectively. In some aspects, the HCDR1, HCDR2, and HCDR3 comprise the amino acid sequences set forth in SEQ ID NOs: 142, 143, and 55, respectively. In some aspects, the HCDR1, HCDR2, and HCDR3 comprise the amino acid sequences set forth in SEQ ID NOs: 142, 144, and 55, respectively. In some aspects, the HCDR1, HCDR2, and HCDR3 comprise the amino acid sequences set forth in SEQ ID NOs: 142, 145, and 55, respectively. In some aspects, the HCDR1, HCDR2, and HCDR3 comprise the amino acid sequences set forth in SEQ ID NOs: 146, 147, and 78, respectively. In some aspects, the HCDR1, HCDR2, and HCDR3 comprise the amino acid sequences set forth in SEQ ID NOs: 148, 149, and 86, respectively. In some aspects, the HCDR1, HCDR2, and HCDR3 comprise the amino acid sequences set forth in SEQ ID NOs: 150, 151, and 93, respectively.

In some aspects, the light chain comprises complementarity determining regions LCDR1, LCDR2, and LCDR3. In some aspects, the VL comprises complementarity determining regions LCDR1, LCDR2, and LCDR3. In some aspects, the LCDR1 comprises the amino acid sequence set forth in SEQ ID NOs: 27, 37, 48, 56, 73, 79, or 94. In some aspects, the LCDR2 comprises the amino acid sequence set forth in SEQ ID NOs: 28, 50, 57, 80, 87, or 95. In some aspects, the LCDR3 comprises the amino acid sequence set forth in SEQ ID NOs: 29, 58, 81, 88, or 96. In some aspects, the LCDR1, LCDR2, and LCDR3 comprise the amino acid sequences set forth in SEQ ID NOs: 27, 28, and 29, respectively. In some aspects, the LCDR1, LCDR2, and LCDR3 comprise the amino acid sequences set forth in SEQ ID NOs: 48, 28, and 29, respectively. In some aspects, the LCDR1, LCDR2, and LCDR3 comprise the amino acid sequences set forth in SEQ ID NOs: 27, 50, and 29, respectively. In some aspects, the LCDR1, LCDR2, and LCDR3 comprise the amino acid sequences set forth in SEQ ID NOs: 56, 57, and 58, respectively. In some aspects, the LCDR1, LCDR2, and LCDR3 comprise the amino acid sequences set forth in SEQ ID NOs: 73, 57, and 58, respectively. In some aspects, the LCDR1, LCDR2, and LCDR3 comprise the amino acid sequences set forth in SEQ ID NOs: 79, 80, and 81, respectively. In some aspects, the LCDR1, LCDR2, and LCDR3 comprise the amino acid sequences set forth in SEQ ID NOs: 56, 87, and 88, respectively. In some aspects, the LCDR1, LCDR2, and LCDR3 comprise the amino acid sequences set forth in SEQ ID NOs: 94, 95, and 96, respectively.

In some aspects, the LCDR1 and LCDR3 comprise the amino acid sequences set forth in SEQ ID NOs: 37 and 29, respectively; and the LCDR2 comprises the amino acid sequence X₁ASX₂X₃X₄X₅, wherein X₁ is R or D; X₂ is H or N; X₃ is L or R; X₄ is Q or A; and X₅ is S or T.

In some aspects, the LCDR1 and LCDR3 comprise the amino acid sequences set forth in SEQ ID NOs: 37 and 29, respectively; and the LCDR2 comprises the amino acid sequence X₁ASX₂X₃X₄X₅, wherein X₁, X₂, X₃, X₄, and X₅ are R, H, L, Q, and S, respectively; or X₁, X₂, X₃, X₄, and X₅ are D, N, R, A, and T, respectively.

In some aspects, the HCDR1, HCDR2, HCDR3, LCDR1, LCDR2, and LCDR3 comprise the amino acid sequences set forth in SEQ ID NOs: 139, 140, 26, 27, 28, and 29, respectively. In some aspects, the HCDR1, HCDR2, HCDR3, LCDR1, LCDR2, and LCDR3 comprise the amino acid sequences set forth in SEQ ID NOs: 139, 140, 26, 27, 50, and 29, respectively.

In some aspects, the antigen-binding site disclosed herein derived from a murine or humanized 02A01, 03A01, 05F10, 11F05, or 04D08 antibodies bind human CD8 in a surface plasmon resonance (SPR) assay with a dissociation constant (KD) value smaller than or equal to (binding affinity greater than or equal to) 10 nM, 15 nM, 20 nM, 25 nM, 30 nM, 40 nM or 50 nM. In some aspects, a bivalent construct of the antigen-binding site disclosed herein derived from a murine or humanized 02A01, 03A01, 05F10, 11F05, or 04D08 antibodies, for example, an anti-CD8 IgG antibody, bind cells expressing human CD8 (e.g., human CD8+ T cells or human T cells comprising a CD8+ population) with a KD value smaller than or equal to binding affinity greater than or equal to 60 pM, 70 pM, 80 pM, 90 pM, 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, or 5 nM. Without wishing to be bound by theory, it has been contemplated that the apparent KD value of a bivalent construct measured by a cell binding assay is lower than the KD value of the same construct measured by SPR, as a result of increased avidity over affinity.

In some aspects, the antigen-binding site disclosed herein derived from a murine or humanized 02A01, 03A01, 05F10, 11F05, or 04D08 antibodies do not cross-compete with OKT8, SK1, 51.1, or MCD8. OKT8 has been found to enhance the on-rates of TCR binding to peptide-MHC class I (pMHC-I), improve pMHC-I tetramer staining on CD8+ T cells, and induce effector function in CD8+ T cells (see, e.g., Clement et al., J. Immunol. (2011) 187(2):654-63). SK1 and MCD8 have been found to inhibit pMHC-I tetramer staining on CD8+ T cells (see, e.g., Wooldridge et al., J. Immunol. (2003) 171:6650-60; Denkberg et al., J. Immunol. (2001) 167:270-76). It is understood that OKT8 is an agonistic antibody and SK1, 51.1, and MCD8 are antagonistic antibodies. Without wishing to be bound by theory, it is contemplated that an antigen-binding site disclosed herein derived from a murine or humanized 02A01, 03A01, 05F10, 11F05, or 04D08 antibodies bind a different epitope on CD8 from OKT8, SK1, 51.1, or MCD8. In some aspects, the present disclosure provides an antigen-binding site (e.g., one derived from a murine or humanized 02A01 antibody) that binds to an epitope corresponding to amino acids 28-32, 34, 48, and 51 of SEQ ID NO: 137. In some aspects, the present disclosure provides an antigen-binding site (e.g., one derived from the murine or humanized 02A01 antibody) that binds to an epitope corresponding to amino acids 49-53, 55, 69, and 72 of SEQ ID NO: 138 (i.e. NCBI Ref. Seq. Acc. No. NP_001139345.1).

In some aspects, the present disclosure provides an antigen-binding site (e.g., one derived from the murine or humanized 03A01 antibody) that binds to an epitope corresponding to amino acids 26, 28-32, 48, 51, 53, 97, and 100 of SEQ ID NO: 137. In some aspects, the present disclosure provides an antigen-binding site (e.g., one derived from the murine or humanized 03A01 antibody) that binds to an epitope corresponding to amino acids 47, 49-53, 69, 72, 74, 118, and 121 of SEQ ID NO: 138 (i.e. NCBI Ref. Seq. Acc. No. NP_001139345.1).

The present disclosure also provides an antigen-binding site (e.g., one derived from a murine or humanized 02A01 or 03A01 antibody) that binds to an epitope corresponding to amino acids 28-32, 48, and 51 of a protein having the amino acid sequence set forth in SEQ ID NO: 137.

The present disclosure also provides an antigen-binding site that cross-competes for binding CD8 (e.g., human CD8) with an antibody or antigen-binding site derived from a murine or humanized 02A01, 03A01, 05F10, 11F05, or 04D08 antibodies, e.g., an antibody or antigen-binding site comprising the VH and VL sequences provided in Tables 1A, 1B, 2A, 2B, 3A, and 3B.

Any one of the antigen-binding sites disclosed herein may be affinity matured. In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. Two or three rounds of mutation and selection using display methods such as phage display can result in antibody fragments with affinities in the low nanomolar range.

An amino acid substitution variation can be introduced into the antigen-binding site by substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domains. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The CD8 binding agent or the antigen-binding sites that bind CD8 disclosed herein is silent and, for example, is lacking a substantial agonistic activity and lacking a substantial antagonistic activity of the antibody or antigen-binding site. In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase an activity of CD8 by more than 10%, 20%, 30%, 40%, 50%, 60%, 70%, 80%, or 90% and/or does not decrease an activity of CD8 by more than 10%, 20%, 30%, 40%, 50%, 60%, 70%, 80%, or 90%. In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase an activity of CD8 by more than about 1.5 fold, 2 fold, 2.5 fold, 3 fold, 4 fold, or 5 fold and/or does not decrease an activity of CD8 by more than about 1.5 fold, 2 fold, 2.5 fold, 3 fold, 4 fold, or 5 fold. The activity of CD8 can be stabilization of a peptide-MHC-TCR complex, activation of a TCR signaling pathway, and/or activation of a transcription factor downstream of TCR signaling, as measured by the assays disclosed herein.

In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase or decrease the stability of a complex by more than about 10%, 20%, 30%, 40%, 50%, 60%, 70%, 80%, or 90%, the complex comprising CD8, a peptide or T cell epitope presented by a human class I MHC tetramer, and a cognate TCR, as measured by the amount of the tetramer bound to a cell expressing the TCR. In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase or decrease the stability of the complex by more than about 1.5 fold, 2 fold, 2.5 fold, 3 fold, 4 fold, or 5 fold.

In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase or decrease T cell activation by more than 10%, 20%, 30%, 40%, 50%, 60%, 70%, 80%, or 90%, as measured by ERK phosphorylation in T cells stimulated by a cognate T cell epitope presented by a human class I MHC tetramer (e.g., the amount of T cells in a population that have phospho-ERK, for example, as assessed by flow cytometry). In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase or decrease the ERK phosphorylation by more than about 1.5 fold, 2 fold, 2.5 fold, 3 fold, 4 fold, or 5 fold.

In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase or decrease T cell activation by more than about 10%, 20%, 30%, 40%, 50%, 60%, 70%, 80%, or 90%, as measured by cytotoxicity of cancer cells caused by T cells, wherein the T cells express a TCR that recognize a T cell epitope presented by a human class I MHC on the surface of the cancer cells. In some aspects, the CD8 binding agent or the antigen-binding site, at a concentration that is at least about 10-fold, 20-fold, 30-fold, 40-fold, 50-fold, 60-fold, 70-fold, 80-fold, 90-fold, or 100-fold higher than the KD value with which the CD8 binding agent or the antigen-binding site binds cells expressing human CD8 (e.g., human CD8+ T cells or human T cells comprising a CD8+ population), does not increase or decrease the cytotoxicity by more than about 1.5 fold, 2 fold, 2.5 fold, 3 fold, 4 fold, or 5 fold.

Other methods can be used to assess the stability of peptide-MHC-TCR complex, the strength of TCR signaling, and the consequent T cell activation. For example, the activation of a transcription factor downstream of TCR signaling (e.g., NFAT) can be assessed using a recombinant construct comprising a NFAT-responsive promoter operably linked to a reporter gene. Exemplary methods are described in Laugel et al. (2007) J. Biol. Chem. 282(33):23799-23810 and in Gaud et al. (2018) Nat. Rev. Immunol. 18(8):485-497, including assessment of degranulation, IFN7 production, TCR downregulation, LCK phosphorylation, and ZAP70 phosphorylation.

In some aspects, the fusion protein comprises an antigen-binding portion that binds CD8. In some aspects, the antigen-binding portion that binds CD8 comprises an Fc Region. In some aspects, the Fc region is modified. In some aspects, the fusion protein disclosed herein lacks an antibody Fc domain. Without wishing to be bound by theory, it is contemplated that the absence of an antibody Fc domain reduces cytotoxicity against the target CD8+ T cells as a result of ADCC, improves tissue penetration, facilitates clearance of circulating protein that is not attached to an immune cell, and improves expression of the protein in vitro or in vivo as a result of the reduced size. The ADCC effector function of an antibody Fc domain is primarily mediated by the CH2 domain. Accordingly, in certain embodiments, the fusion protein lacks an antibody Fc CH2 domain.

In some aspects, the fusion protein disclosed herein comprises an antibody Fc domain having low or no ADCC effector function. Without wishing to be bound by theory, it is contemplated that the presence of an Fc domain (e.g., an Fc domain that binds FcRn) may increase the serum half-life of the fusion protein and the low level or absence of ADCC effector function reduces the risk of killing the target CD8+ T cells. While the longer serum half-life could in theory increase both efficacy and toxicity, it is contemplated that the potential toxicity is mitigated by specific targeting of the fusion protein to CD8-expressing cells. ADCC is mediated by binding to Fcγ receptors. Accordingly, in certain embodiments, the Fc domain incorporates one or more mutations or modifications, in either or both Fc polypeptide chains, that alter the binding to an Fcγ receptor (e.g., FcγRI/CD64, FcγRIIA/CD32A, FcγRIIB/CD32B, FcγRIIIIA/CD16, or FcγRIIIB).

In some aspects, the antibody Fc domain is an IgG1, IgG2, IgG3, or IgG4 Fc domain from human or another mammal, such as rabbit, dog, cat, mouse, or horse. In certain embodiments, the antibody Fc domain is a human IgG1, IgG2, IgG3, or IgG4 Fc domain. Fc domains of human IgG1 and IgG3 are known to have ADCC and CDC effector functions. In some aspects, the fusion protein disclosed herein comprises an antibody Fc domain or a fragment thereof in which an ADCC effector function is reduced by at least about 50%, 60%, 70%, 80%, 90%, 91%, 92%, 93%, 94%, 95%, 96%, 97%, 98%, or 99% relative to a wild-type human IgG1 Fc domain having the amino acid sequence set forth in SEQ ID NO: 97. In some aspects, the fusion protein disclosed herein comprises an antibody Fc domain or a fragment thereof in which a CDC effector function is reduced by at least about 50%, 60%, 70%, 80%, 90%, 91%, 92%, 93%, 94%, 95%, 96%, 97%, 98%, or 99% relative to a wild-type human IgG1 Fc domain having the amino acid sequence set forth in SEQ ID NO: 97.

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction. All the amino acid positions in antibody Fc domains in this application are numbered according to the EU numbering system (see Edelman et al., (1969) Proc. Natl. Acad. Sci. USA 63:78-85; Kabat et al., Sequences of Proteins of Immunological Interest (5th ed. 1991)).

Mutations in IgG Fc domain and their impact on ADCC and CDC effector functions have been characterized (see, e.g., Wang et al., (2018) Protein Cell 9(1):63-73). In some aspects, the fusion protein comprising an antibody (e.g., IgG, e.g., human IgG1) Fc domain comprising one or more mutations that reduce ADCC and/or CDC effector functions. Exemplary Fc domain mutations that reduce ADCC and CDC effector functions are disclosed in U.S. Pat. No. 11,084,863. In some aspects, the antibody Fc domain differs from the wild-type human IgG1 Fc domain at one or more positions selected from R292, S298, L234, L235, G237, N297, S298, A327, P329, A330, P331, and K414. In some aspects, the antibody Fc domain differs from the human IgG1 Fc domain at one or more positions selected from L234, L235, G237, A327, P329, A330, and P331. In some aspects, the antibody Fc domain differs from the human IgG1 Fc domain at one or more positions selected from R292, S298, and K414. In some aspects, the antibody Fc domain differs from the human IgG1 Fc domain at: (a) positions L234 and L235; (b) positions L234, L235, and P329; or (c) positions L234, L235, A327, A330, and P331.

In some aspects, the one or more mutations are selected from L234A, L235A, L235E, N297A, N297G, N297Q, S298A, A327G, P329A, P329G, A330S, and P331S. In some aspects, the fusion protein comprises a human IgG1 Fc comprising L234A and L235A substitutions. In some aspects, the fusion protein comprises a human IgG1 Fc comprising L234A, L235A, and P329G or P329A substitutions. In some aspects, the fusion protein comprises a human IgG1 Fc comprising N297A substitution. In some aspects, the fusion protein comprises a human IgG1 Fc comprising L234A, L235A, and N297A substitutions In some aspects, the fusion protein comprises a human IgG1 Fc comprising L234A, L235A, N297A, and P329G or P329A substitutions. In some aspects, the fusion protein comprises a human IgG1 Fc comprising L234A, L235A, A327G, A330S, and P331S substitutions. In some aspects, the fusion protein comprises a human IgG1 Fc comprising L234A, L235A, N297A, A327G, A330S, and P331S substitutions.

Fc domains of human IgG4 and IgG2 are known to have little or no ADCC effector function. In some aspects, the fusion protein comprises a human IgG4 Fc domain or a fragment thereof. In some aspects, the IgG4 Fc domain comprises a S228P substitution. In some aspects, the fusion protein comprises a human IgG2 Fc domain or a fragment thereof.

Where the format of a Fc-containing fusion protein disclosed herein is asymmetric, for example, in the Fab-Fc-immunomodulator (LC) format or the IgG-immunomodulator (1×HC) format, one or more mutations can be introduced into the Fc domain to promote heterodimerization. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In some aspects, at least one amino acid substitution can be made at K392, K370, K409, and/or K439 of the first Fc polypeptide chain, and at least one amino acid substitution can be made at D399, E356, and/or E357 of the second polypeptide chain, where the amino acid residues in the first Fc polypeptide chain is replaced by any known negatively-charged amino acid, and the amino acid residue in the second Fc polypeptide chain is replaced by any known positively-charged amino acid.

In some aspects, the amino acid sequence of one polypeptide chain of the antibody constant region differs from the amino acid sequence of an IgG1 constant region at position T366. In some aspects, the amino acid sequence of the other polypeptide chain of the antibody constant region differs from the amino acid sequence of an IgG1 constant region at one or more positions selected from the group consisting of T366, L368 and Y407.

In some aspects, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411. In some aspects, the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some aspects, the amino acid sequence of one polypeptide chain of the antibody constant region differs from the amino acid sequence of an IgG1 constant region at one or more positions selected from the group consisting of L351, D399, S400 and Y407. In some aspects, the amino acid sequence of the other polypeptide chain of the antibody constant region differs from the amino acid sequence of an IgG1 constant region at one or more positions selected from the group consisting of T366, N390, K392, K409 and T411.

In some aspects, the amino acid sequence of one polypeptide chain of the antibody constant region differs from the amino acid sequence of an IgG1 constant region at one or more positions selected from the group consisting of Q347, Y349, K360, and K409. In some aspects, the amino acid sequence of the other polypeptide chain of the antibody constant region differs from the amino acid sequence of an IgG1 constant region at one or more positions selected from the group consisting of Q347, E357, D399 and F405.

In some aspects, the amino acid sequence of one polypeptide chain of the antibody constant region differs from the amino acid sequence of an IgG1 constant region at one or more positions selected from the group consisting of K370, K392, K409 and K439. In some aspects, the amino acid sequence of the other polypeptide chain of the antibody constant region differs from the amino acid sequence of an IgG1 constant region at one or more positions selected from the group consisting of D356, E357 and D399.

In some aspects, the amino acid sequence of one polypeptide chain of the antibody constant region differs from the amino acid sequence of an IgG1 constant region at one or more positions selected from the group consisting of L351, E356, T366 and D399. In some aspects, the amino acid sequence of the other polypeptide chain of the antibody constant region differs from the amino acid sequence of an IgG1 constant region at one or more positions selected from the group consisting of Y349, L351, L368, K392 and K409.

In some aspects, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V. This set of substitutions are referred to as “knobs-in-holes,” wherein the polypeptide chain comprising a T366W substitution has a “knob” and the polypeptide chain comprising T366S, L368A, and Y407V substitutions has a “hole.” In certain embodiments, the polypeptide chain linked to an immunomodulator (e.g., a cytokine, e.g., IL-2) comprises the “knob” substitution and the polypeptide chain not linked to an immunomodulator (e.g., a cytokine, e.g., IL-2) comprises the “hole” substitutions.

Alternatively, amino acid substitutions could be selected from the following sets of substitutions shown in Table 4 below. Additional exemplary Fc domain substitutions that promote heterodimerization are disclosed in U.S. Pat. No. 11,084,863.

TABLE 4
Fc Heterodimerization Mutations

| | First Fc Polypeptide | Second Fc Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |
| Set 16 | K409W | D399V/F405T |
| Set 17 | Y349S | E357W |
| Set 18 | K360E | Q347R |
| Set 19 | K360E/K409W | Q347R/D399V/F405T |
| Set 20 | Q347E/K360E/K409W | Q347R/D399V/F405T |
| Set 21 | Y349S/K409W | E357W/D399V/F405T |
| Set 22 | T366K/L351K | L351D/L368E |
| Set 23 | T366K/L351K | L351D/Y349E |
| Set 24 | T366K/L351K | L351D/Y349D |
| Set 25 | T366K/L351K | L351D/Y349E/L368E |
| Set 26 | T366K/L351K | L351D/Y349D/L368E |
| Set 27 | E356K/D399K | K392D/K409D |
| Set 28 | L351Y, D399R, D399K, | T366V, T366I, T366L, T366M, |
| | S400K, S400R, Y407A, | N390D, N390E, K392L, K392M, |
| | Y407I, Y407V | K392V, K392F K392D, K392E, |
| | | K409F, K409W, T411D and |
| | | T411E |
| Set 29 | T350V, L351Y, F405A, | T350V, T366L, K392L, and |
| | and Y407V | T394W |
| Set 30 | T366W | T366A/L368A/Y407V |

In some aspects, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

Unless indicated otherwise, the amino acid substitutions described above are identified in the context of human IgG1. It is understood that corresponding substitutions are also contemplated in the context of human IgG2, IgG3, and IgG4. For example, the amino acid residue at position 234 of human IgG4 is F, whereas the amino acid residue at position 234 of human IgG1 is L. Where an L234A substitution is described in the context of human IgG1, the F234A substitution in the context of human IgG4 is also contemplated.

It is understood that where the two Fc polypeptide chains, either referred to as the first polypeptide chain and the second polypeptide chain or as one polypeptide chain and the other polypeptide chain, contain different amino acid sequences or mutations, either chain can be linked to any other domain of the fusion proteins disclosed herein. Where an exemplary fusion protein is described to contain a first set of mutations in a given first Fc polypeptide chain and a second set of mutations in the second Fc polypeptide chain, the reverse construct, in which the first Fc polypeptide chain contains the second set of mutations and the second Fc polypeptide chain contains the first set of mutations, is also contemplated.

### B. IL-2 Portion

In some aspects, the IL-2 portion in the fusion protein comprises an IL-2 protein. In some aspects, the IL-2 protein comprises a sequence that is found naturally (wild type). In some aspects, the IL-2 protein useful for the fusion protein comprises one or more amino acids that are different from the wild type IL-2. In some aspects, the IL-2 protein is a mature sequence.

In some aspects, the IL-2 protein comprises the amino acid sequence of SEQ ID NO: 110. In some aspects, the IL-2 protein comprises an amino acid sequence from Table 5 below.

TABLE 5
IL-2 protein sequences

| Protein Name | Amino Acid Sequence |
|---|---|
| Human IL2 | APTSSSTKKTQLQLEHLLLDLQMILNGINNYKNPKLTRMLTFKFYMPKKATELKHL |
| | QCLEEELKPLEEVLNLAQSKNFHLRPRDLISNINVIVLELKGSETTFMCEYADETA |
| | TIVEFLNRWITFCQSIISTLT (SEQ ID NO: 110) |

In some aspects, the IL-2 protein comprises an amino acid sequence having at least about 60% (e.g., at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100%) sequence identity to the amino acid sequence set forth in SEQ ID NO: 110.

In some aspects, at least one of the IL-2 protein exhibits a decreased binding to CD25, relative to the wild-type IL-2. In some aspects, the at least one of the IL-2 protein comprises an amino acid that is different from the wild-type IL-2, wherein the IL-2 protein has a decreased binding to CD25 (“substituted amino acid”). In some aspects, the substituted amino acid is at residues 34 to 45, residues 60 to 72, and/or residues 104 to 115 corresponding to the amino acid sequence set forth in SEQ ID NO: 110 (wild type IL2). In some aspects, the substituted amino acid is at R38, K35, F42, K43, Y45, E61, E62, P65, L72, or any combination thereof, corresponding to SEQ ID NO: 110. In some aspects, the substituted amino acid is at P34, K35, L36, T37, R38, L40, T41, F42, K43, Y45, E60, E61, K64, P65, E68, L72, M104, Y107, A108, D109, T111, T113, V115, or any combination thereof, corresponding to the amino acid sequence set forth in SEQ ID NO: 110. In some aspects, the substituted amino acid is at residues K35, L36, T37, K64, or any combination thereof, corresponding to SEQ ID NO: 110. In some aspects, the substituted amino acid is at residues K35, L36, T37, K64, P34, L40, T41, K43, Y45, K64, M104, Y107, T113, V115, or any combination thereof, corresponding to SEQ ID NO: 110. In some aspects, the substituted amino acid is at residues K35, L36, T37, K64, P34, L40, T41, K43, Y45, K64, M104, Y107, T113, V115, R38, F42, P65, E68, L72, or any combination thereof, corresponding to SEQ ID NO: 110. In some aspects, the substituted amino acid is at residues K35, L36, T37, K64, P34, L40, T41, K43, Y45, K64, M104, Y107, T113, V115, R38, F42, P65, E68, L72, L40, E61, A108, D109, T111, or any combination thereof, corresponding to SEQ ID NO: 110.

In some aspects, the substituted amino acid is A at residue 3 (T3A), E at residue 34 (P34E), E at residue 35 (K35E), N at residue 35 (K35N), W at residue 36 (L36W), A at residue 36 (L36A), N at residue 36 (L36N), E at residue 37 (T37E), T at residue 38 (R38T), F at residue 38 (R38F), H at residue 38 (R38H), W at residue 40 (L40W), G at residue 40 (L40G), D at residue 40 (L40D), A at residue 41 (T41A), Q at residue 41 (T41Q), E at residue 41 (T41E), Y at residue 42 (F42Y), S at residue 43 (K43S), R at residue 43 (K43R), Q at residue 43 (K43Q), L at residue 45 (Y45L), G at residue 45 (Y45G), F at residue 45 (Y45F), R at residue 60 (E60R), N at residue 61 (E61N), G at residue 61 (E61G), Y at residue 61 (E61Y), G at residue 64 (K64G), D at residue 64 (K64D), A at residue 64 (K64A), S at residue 65 (P65S), D at residue 65 (P65D), Y at residue 68 (E68Y), A at residue 68 (E68A), T at residue 68 (E68T), T at residue 72 (L72T), Q at residue 72 (L72Q), W at residue 72 (L72W), K at residue 104 (M104K), F at residue 107 (Y107F), L at residue 107 (Y107L), Q at residue 107 (Y107Q), H at residue 107 (Y107H), Y at residue 108 (A108Y), R at residue 109 (D109R), L at residue 111 (T111L), Q at residue 111 (T111Q), R at residue 31114 (T111R), G at residue 113 (T113G), E at residue 113 (T113E), Y at residue 115 (V115Y), H at residue 115 (V115H), A at residue 125 (C125A), or any combination thereof corresponding to SEQ ID NO: 110.

In some aspects, the substituted amino acid is A at residue 38 (R38A), K at residue 38 (R38K), A at residue 35 (K35A), A at residue 42 (F42A), I at residue 42 (F42I), Q at residue 42 (F42Q), K at residue 42 (F42K), A at residue 43 (K43A), E at residue 43 (K43E), N at residue 43 (K43N), A at residue 45 (Y45A), S at residue 45 (Y45S), N at residue 45 (Y45N), E at residue 45 (Y45E), R at residue 45 (Y45R), Q at residue 61 (E61Q), A at residue 61 (E61A), K at residue 61 (E61K), A at residue 62 (E62A), K at residue 62 (E62K), L at residue 62 (E62L), V at residue 62 (E62V), L at residue 65 (P65L), H at residue 65 (P65H), G at residue 72 (L72G), Q at residue 72 (L72Q), or any combination thereof corresponding to SEQ ID NO: 110.

In some aspects, the substituted amino acid is K35E, L36A, L36N, T37E, K64G, or any combination thereof corresponding to SEQ ID NO: 110. In some aspects, the substituted amino acid is K35E, L36A, L36N, T37E, K64G, P34E, L36W, L40D, T41A, K43Q, Y45L, K64D, M104K, Y107F, T113G, V115H, or any combination thereof corresponding to SEQ ID NO: 110.

In some aspects, the substituted amino acid is K35E, L36A, L36N, T37E, K64G, P34E, L36W, L40D, T41A, K43Q, Y45L, K64D, M104K, Y107F, T113G, V115H, K35N, R38T, R38F, R38H, T41Q, F42Y, K43S, K43R, Y45F, K64A, P65S, P65D, E68A, L72W, V115Y, or any combination thereof corresponding to SEQ ID NO: 110.

In some aspects, the substituted amino acid is K35E, L36A, L36N, T37E, K64G, P34E, L36W, L40D, T41A, K43Q, Y45L, K64D, M104K, Y107F, T113G, V115H, K35N, R38T, R38F, R38H, T41Q, F42Y, K43S, K43R, Y45F, K64A, P65S, P65D, E68A, L72W, V115Y, L40W, T41E, Y45G, E61N, E61G, E68Y, E68T, L72T, A108Y, D109R, T111L, T111Q, or any combination thereof corresponding to SEQ ID NO: 110.

In some aspects, at least one of the IL-2 proteins has an amino acid that is different from the wild-type IL-2, wherein the IL-2 protein has altered binding to at least one IL-2 receptor relative to wild-type IL-2, wherein the IL-2 receptor is selected from the group consisting of CD25, CD122, and CD132.

In some aspects, the present disclosure provides a novel IL-2 protein that comprises at least one amino acid that is different from the wild-type IL-2, wherein the IL-2 protein has altered binding to at least one IL-2 receptor relative to wild-type IL-2, wherein the IL-2 receptor is selected from the group consisting of CD25, CD122, and CD132. Therefore, in some aspects the novel IL-2 protein has reduced binding to CD25, CD122, and/or CD132. In some aspects, the novel IL-2 protein has increased binding to CD25, CD122, and/or CD132.

In some aspects, the novel IL-2 protein comprises at least one amino acid different from the wild-type IL-2 (substituted amino acid), wherein the substituted amino acid comprises A at residue 38 (R38A), K at residue 38 (R38K), A at residue 35 (K35A), A at residue 42 (F42A), I at residue 42 (F42I), Q at residue 42 (F42Q), K at residue 42 (F42K), A at residue 43 (K43A), E at residue 43 (K43E), N at residue 43 (K43N), A at residue 45 (Y45A), S at residue 45 (Y45S), N at residue 45 (Y45N), E at residue 45 (Y45E), R at residue 45 (Y45R), Q at residue 61 (E61Q), A at residue 61 (E61A), K at residue 61 (E61K), A at residue 62 (E62A), K at residue 62 (E62K), L at residue 62 (E62L), V at residue 62 (E62V), L at residue 65 (P65L), H at residue 65 (P65H), G at residue 72 (L72G), Q at residue 72 (L72Q), or any combination thereof corresponding to SEQ ID NO: 110.

In some aspects, the novel IL-2 protein comprises at least one substituted amino acid comprising K35E, L36A, L36N, T37E, K64G, or any combination thereof corresponding to SEQ ID NO: 110. In some aspects, the substituted amino acid is K35E, L36A, L36N, T37E, K64G, P34E, L36W, L40D, T41A, K43Q, Y45L, K64D, M104K, Y107F, T113G, V115H, or any combination thereof corresponding to SEQ ID NO: 110.

In some aspects, the novel IL-2 protein comprises at least one substituted amino acid comprising K35E, L36A, L36N, T37E, K64G, P34E, L36W, L40D, T41A, K43Q, Y45L, K64D, M104K, Y107F, T113G, V115H, K35N, R38T, R38F, R38H, T41Q, F42Y, K43S, K43R, Y45F, K64A, P65S, P65D, E68A, L72W, V115Y, or any combination thereof corresponding to SEQ ID NO: 110.

In some aspects, the novel IL-2 protein comprises at least one substituted amino acid is K35E, L36A, L36N, T37E, K64G, P34E, L36W, L40D, T41A, K43Q, Y45L, K64D, M104K, Y107F, T113G, V115H, K35N, R38T, R38F, R38H, T41Q, F42Y, K43S, K43R, Y45F, K64A, P65S, P65D, E68A, L72W, V115Y, L40W, T41E, Y45G, E61N, E61G, E68Y, E68T, L72T, A108Y, D109R, T111L, T111Q, or any combination thereof corresponding to SEQ ID NO: 110.

### C. Linkers

In some aspects, the fusion protein comprises a linker. In some aspects, the IL-2 portion of the fusion protein is linked to the antigen-binding portion of the fusion protein by a linker. In some aspects, the linker comprises the amino acid sequence set forth in SEQ ID NO: 107.

The fusion protein disclosed herein includes multiple components, which can be linked to each other by a peptide bond or a linker (e.g., peptide linker). In certain embodiments, the peptide linker does not comprise any multimerization or polymerization activity. A peptide linker disclosed herein can be used to link a VH and a VL in a scFv. A peptide linker disclosed herein can also be used to link between a CD8-binding site, an immunomodulator (e.g., a cytokine, e.g., an immunostimulatory cytokine), and/or an antibody Fc domain. Exemplary peptide linkers are described in U.S. Pat. Nos. 4,751,180 and 4,935,233 and International Application Publication No. WO198809344A1.

A peptide linker in a given fusion protein may have an optimized length and/or amino acid composition. In some aspects, a peptide linker is short, consisting of 1, 2, 3, 4, 5, 6, 7, 8, 9, 10, 11, 12, 13, or 14 amino acid residues. Thus, in some aspects, the peptide linker consists of about 12 or fewer amino acid residues. In some aspects, a peptide linker is longer, consisting of 15, 20, 25, or more amino acid residues. In some aspects, a peptide linker consists of about 3 to about 15, for example 8, 9 or 10 amino acid residues.

Regarding the amino acid composition of a peptide linker, peptides are selected with properties that confer flexibility to the fusion proteins disclosed herein, do not interfere with the CD8-binding site and the immunomodulator, and resist cleavage from proteases. For example, glycine and serine residues generally provide protease resistance. Examples of the linkers suitable for linking the domains in the fusion proteins include but are not limited to (GS)n, (GGS)n, (GGGS)n, (GGSG)n, (GGSGG)n, and (GGGGS)n, wherein n is 1, 2, 3, 4, 5, 6, 7, 8, 9, 10, 11, 12, 13, 14, 15, 16, 17, 18, 19, or 20. In some embodiments, a peptide linker is selected from the peptide sequences listed in Table 6.

In some aspects, the fusion protein disclosed herein comprises an immunomodulator (e.g., IL-2) linked to the C-terminus of a Fab light chain by a peptide linker comprising the amino acid sequence set forth in SEQ ID NO: 98, 99, 100, 101, 102, 103, 104, 105, 106, 107, 108, or 109. In some aspects, the fusion protein disclosed herein comprises an immunomodulator (e.g., IL-2) linked to the C-terminus of a Fab light chain by a peptide linker comprising the amino acid sequence set forth in SEQ ID NO: 107.

In some aspects, the fusion protein disclosed herein comprises an immunomodulator (e.g., IL-2) linked to the C-terminus of a Fc domain polypeptide by a peptide linker comprising the amino acid sequence set forth in SEQ ID NO: 98, 99, 100, 101, 102, 103, 104, 105, 106, 107, 108, or 109. In some aspects, the fusion protein disclosed herein comprises an immunomodulator (e.g., IL-2) linked to the C-terminus of a Fc domain polypeptide by a peptide linker comprising the amino acid sequence set forth in SEQ ID NO: 107.

TABLE 6
Exemplary Peptide Linkers

| Linker | Amino Acid Sequence |
|---|---|
| (GS)₁₀ | GSGSGSGSGSGSGSGSGSGS (SEQ ID NO: 101) |
| (GGS)₁₀ | GGSGGSGGSGGSGGSGGSGGSGGSGGSGGS (SEQ ID NO: 102) |
| (GGGS)₁₀ | GGGSGGGSGGGSGGGSGGGSGGGSGGGSGGGSGGGSGGGS (SEQ ID NO: 103) |
| (GGSG)₁₀ | GGSGGGSGGGSGGGSGGGSGGGSGGGSGGGSGGGSGGGSG (SEQ ID NO: 104) |
| (GGSGG)₁₀ | GGSGGGGSGGGGSGGGGSGGGGSGGGGSGGGGSGGGGSGGGGSGGGGSGG |
| | (SEQ ID NO: 105) |
| (GGGGS)₁₀ | GGGGSGGGGSGGGGSGGGGSGGGGSGGGGSGGGGSGGGGSGGGGSGGGGS |
| | (SEQ ID NO: 106) |
| (GGGGS)₄ | GGGGSGGGGSGGGGSGGGGS (SEQ ID NO: 99) |
| (GGGGS)₃ | GGGGSGGGGSGGGGS (SEQ ID NO: 107) |
| (GGGGS)₂₀ | GGGGSGGGGSGGGGSGGGGSGGGGSGGGGSGGGGSGGGGSGGGGSGGGGSGGGGSGGGGSGGGGSG |
| | GGGSGGGGSGGGGSGGGGSGGGGSGGGGSGGGGS (SEQ ID NO: 108) |
| (GGSGG)₂₀ | GGSGGGGSGGGGSGGGGSGGGGSGGGGSGGGGSGGGGSGGGGSGGGGSGGGGSGGGGSGGGGSGGG |
| | GSGGGGSGGGGSGGGGSGGGGSGGGGSGGGGSGG (SEQ ID NO: 109) |
| (GGGS)₄ | GGGSGGGSGGGSGGGS (SEQ ID NO: 98) |
| GGGS- | GGGSGGGGSGGGGSGGGGS (SEQ ID NO: 100) |
| (GGGGS)₃ | |

### D. Exemplary CD8 Antibody-IL2 Fusion Proteins

In some aspects, the fusion protein comprises a heavy chain. In some aspects, the heavy chain comprises the amino acid sequence set forth in SEQ ID NO: 1, 3, or 5. In some aspects, the antigen-binding portion comprises a heavy chain comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 1. In some aspects, the antigen-binding portion comprises a heavy chain comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3. In some aspects, the antigen-binding portion comprises a heavy chain comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 5.

In some aspects, the fusion protein comprises a light chain. In some aspects, the light chain comprises the amino acid sequence set forth in SEQ ID NO: 2, 4, 6, or 47. In some aspects, the antigen-binding portion comprises a light chain comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 2. In some aspects, the antigen-binding portion comprises a light chain comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 4. In some aspects, the antigen-binding portion comprises a light chain comprising an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 6.

In some aspects, a fusion protein of the disclosure comprises two polypeptide chains. In some aspects, the first polypeptide chain comprises the amino acid sequence set forth in SEQ ID NO: 8 and the second polypeptide chain comprises the amino acid sequence set forth in SEQ ID NO: 10. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 8. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 10.

In some aspects, a fusion protein of the disclosure comprises two polypeptide chains. In some aspects, the first polypeptide chain comprises the amino acid sequence set forth in SEQ ID NO: 3 and the second polypeptide chain comprises the amino acid sequence set forth in SEQ ID NO: 17. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 3. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 17.

In some aspects, a fusion protein of the disclosure comprises two polypeptide chains. In some aspects, the first polypeptide chain comprises the amino acid sequence as set forth in SEQ ID NO: 5 and the second polypeptide chain comprises the amino acid sequence as set forth in SEQ ID NO: 20. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 5. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 20.

In some aspects, a fusion protein of the disclosure comprises two polypeptide chains. In some aspects, the first polypeptide chain comprises the amino acid sequence set forth in SEQ ID NO: 1 and the second polypeptide chain comprises the amino acid sequence set forth in SEQ ID NO: 10. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 1. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 10.

In some aspects, a fusion protein of the disclosure comprises three polypeptide chains. In some aspects, the first polypeptide chain comprises the amino acid sequence set forth in SEQ ID NO: 7, the second polypeptide chain comprises the amino acid sequence set forth in SEQ ID NO: 125, and the third polypeptide chain comprises the amino acid sequence set forth in SEQ ID NO: 10. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 7. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 125.

In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 10.

In some aspects, a fusion protein of the disclosure comprises three polypeptide chains. In some aspects, the first polypeptide chain comprises the amino acid sequence set forth in SEQ ID NO: 11, the second polypeptide chain comprises the amino acid sequence set forth in SEQ ID NO: 15, and the third polypeptide chain comprises the amino acid sequence set forth in SEQ ID NO: 4. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 11. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 15. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 4.

In some aspects, a fusion protein of the disclosure comprises three polypeptide chains. In some aspects, the first polypeptide chain comprises the amino acid sequence set forth in SEQ ID NO: 21, the second polypeptide chain comprises the amino acid sequence set forth in SEQ ID NO: 19 and the third polypeptide chain comprises the amino acid sequence set forth in SEQ ID NO: 6. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 21. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 19. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 6.

In some aspects, a fusion protein of the disclosure comprises two polypeptide chains. In some aspects, the first polypeptide chain comprises the amino acid sequence set forth in SEQ ID NO: 9 and the second polypeptide chain comprises the amino acid sequence set forth in SEQ ID NO: 2. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity tical to SEQ ID NO: 9. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 2.

In some aspects, a fusion protein of the disclosure comprises three polypeptide chains. In some aspects, the first polypeptide chain comprises the amino acid sequence set forth in SEQ ID NO: 14, the second polypeptide chain comprises the amino acid sequence set forth in SEQ ID NO: 16, and the third polypeptide chain comprises the amino acid sequence set forth in SEQ ID NO: 4. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 14. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 16. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 4.

In some aspects, a fusion protein of the disclosure comprises two polypeptide chains. In some aspects, the first polypeptide chain comprises the amino acid sequence set forth in SEQ ID NO: 13 and the second polypeptide chain comprises the amino acid sequence set forth in SEQ ID NO: 4. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 13. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 4.

In some aspects, a fusion protein of the disclosure comprises two polypeptide chains. In some aspects, the first polypeptide chain comprises the amino acid sequence set forth in SEQ ID NO: 12 and the second polypeptide chain comprises the amino acid sequence set forth in SEQ ID NO: 18. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 12. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 18.

In some aspects, a fusion protein of the disclosure comprises two polypeptide chains. In some aspects, the first polypeptide chain comprises the amino acid sequence set forth in SEQ ID NO: 111 and the second polypeptide chain comprises the amino acid sequence set forth in SEQ ID NO: 112. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 111. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 112.

In some aspects, a fusion protein of the disclosure comprises two polypeptide chains. In some aspects, the first polypeptide chain comprises the amino acid sequence set forth in SEQ ID NO: 116 and the second polypeptide chain comprises the amino acid sequence set forth in SEQ ID NO: 117. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 116. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 117.

In some aspects, a fusion protein of the disclosure comprises two polypeptide chains. In some aspects, the first polypeptide chain comprises the amino acid sequence set forth in SEQ ID NO: 112 and the second polypeptide chain comprises the amino acid sequence set forth in SEQ ID NO: 115. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 112. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 115.

In some aspects, a fusion protein of the disclosure comprises two polypeptide chains. In some aspects, the first polypeptide chain comprises the amino acid sequence set forth in SEQ ID NO: 120 and the second polypeptide chain comprises the amino acid sequence set forth in SEQ ID NO: 117. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 120. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 117.

In some aspects, a fusion protein of the disclosure comprises two polypeptide chains. In some aspects, the first polypeptide chain comprises the amino acid sequence set forth in SEQ ID NO: 121 and the second polypeptide chain comprises the amino acid sequence set forth in SEQ ID NO: 115. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 121. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 115.

In some aspects, a fusion protein of the disclosure comprises two polypeptide chains. In some aspects, the first polypeptide chain comprises the amino acid sequence set forth in SEQ ID NO: 123 and the second polypeptide chain comprises the amino acid sequence set forth in SEQ ID NO: 117. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 123. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 117.

In some aspects, a fusion protein of the disclosure comprises three polypeptide chains. In some aspects, the first polypeptide chain comprises the amino acid sequence set forth in SEQ ID NO: 112, the second polypeptide chain comprises the amino acid sequence set forth in SEQ ID NO: 113, and the third polypeptide chain comprises the amino acid sequence set forth in SEQ ID NO: 114. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 112. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 113. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 114.

In some aspects, a fusion protein of the disclosure comprises three polypeptide chains. In some aspects, the first polypeptide chain comprises the amino acid sequence set forth in SEQ ID NO: 117, the second polypeptide chain comprises the amino acid sequence set forth in SEQ ID NO: 118, and the third polypeptide chain comprises the amino acid sequence set forth in SEQ ID NO: 119. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 117. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 118. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 119.

In some aspects, a fusion protein of the disclosure comprises three polypeptide chains. In some aspects, the first polypeptide chain comprises the amino acid sequence set forth in SEQ ID NO: 122, the second polypeptide chain comprises the amino acid sequence set forth in SEQ ID NO: 113, and the third polypeptide chain comprises the amino acid sequence set forth in SEQ ID NO: 117. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 122. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 113. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 117.

In some aspects, a fusion protein of the disclosure comprises three polypeptide chains. In some aspects, the first polypeptide chain comprises the amino acid sequence set forth in SEQ ID NO: 114, the second polypeptide chain comprises the amino acid sequence set forth in SEQ ID NO: 117, and the third polypeptide chain comprises the amino acid sequence set forth in SEQ ID NO: 118. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 114. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 117. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 118.

In some aspects, a fusion protein of the disclosure comprises three polypeptide chains. In some aspects, the first polypeptide chain comprises the amino acid sequence as set forth in SEQ ID NO: 124, the second polypeptide chain comprises the amino acid sequence set forth in SEQ ID NO: 113, and the third polypeptide chain comprises the amino acid sequence set forth in SEQ ID NO: 117. In some aspects, the first polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 124. In some aspects, the second polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 113. In some aspects, the third polypeptide chain comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity to SEQ ID NO: 117.

In some aspects, the fusion protein of the disclosure has one or more of the following properties: a) activates pSTAT5 in CD8+ T cells with 10-fold or greater potency as compared to activation of pSTAT5 in NK cells; b) activates pSTAT5 in CD8+ T cells at similar or greater potency than Tregs; or c) preferentially induces proliferation of CD8+ T cells over Tregs and NK cells.

In some aspects, the fusion protein activates pSTAT5 in CD8+ T cells with 10-fold or greater potency as compared to activation of pSTAT5 in NK cells.

In some aspects, the fusion protein activates pSTAT5 in CD8+ T cells at similar or greater potency than Tregs.

In some aspects, the fusion protein preferentially induces proliferation of CD8+ T cells over Tregs and NK cells.

In some aspects, the fusion protein comprises an amino acid sequence from Table 7 and an amino acid sequence from Table 8. Table 9 lists examples of the fusion proteins comprising an antigen-binding site that binds CD8 and an IL-2 protein.

TABLE 7
Exemplary Heavy Chains

| Heavy Chain | Amino Acid Sequence |
|---|---|
| YTS105 Heavy | EVQLKESGPGLVQPSQTLSLTCTVSGFSLTSNSVHWVRQPPGKGLEWMGGIWGDGDTDYNSALKSRL |
| Chain | SISRDTSKNQVFLKMNSLQTDDTAIYFCTPLIGSWYFDFWGPGTMVTASSASTKGPSVFPLAPSSKS |
| | TSGGTAALGCLVKDYFPEPVTVSWNSGALTSGVHTFPAVLQSSGLYSLSSVVTVPSSSLGTQTYICN |
| | VNHKPSNTKVDKKVEPKSC (SEQ ID NO: 1) |
| YTS169 Heavy | EVKLQESGGGLVQPGRSLKLSCAASGFNFNDYWMGWVRQAPGKGLEWIGEINKDSSTINYTPSLKDK |
| Chain | FTISRDNAQNTLYLQMSKLGSEDTAIYYCARARGMMVLIIPHYFDYWGQGVMVTVSSAKTTAPSVYP |
| | LAPVCGDTTGSSVTLGCLVKGYFPEPVTLTWNSGSLSSGVHTFPAVLQSDLYTLSSSVTVTSSTWPS |
| | QSITCNVAHPASSTKVDKKIEPRGPTIKPCPPCKCPAPNAAGGPSVFIFPPKIKDVLMISLSPIVTC |
| | VVVDVSEDDPDVQISWFVNNVEVHTAQTQTHREDYNSTLRVVSALPIQHQDWMSGKEFKCKVNNKDL |
| | PAPIERTISKPKGSVRAPQVYVLPPPEEEMTKKQVTLTCMVTDFMPEDIYVEWTNNGKTELNYKNTE |
| | PVLDSDGSYFMYSKLRVEKKNWVERNSYSCSVVHEGLHNHHTTKSFSRTPGK (SEQ ID NO: 3) |
| Humanized | EVQLVQSGAEVKKPGATVKISCKASGYTFTDFTMHWVQQAPGKGLEWIGLVNPNNGGNIYAEKFQGR |
| 02A01-VH6- | VTLTVDTSTSTAYMELSSLRSEDTAVYYCGRVPLYTSHGMDYWGQGTLVTVSSASTKGPSVFPLAPS |
| Full IgG Heavy | SKSTSGGTAALGCLVKDYFPEPVTVSWNSGALTSGVHTFPAVLQSSGLYSLSSVVTVPSSSLGTQTY |
| Chain | ICNVNHKPSNTKVDKKVEPKSCDKTHTCPPCPAPEAAGGPSVFLFPPKPKDTLMISRTPEVTCVVVD |
| | VSHEDPEVKFNWYVDGVEVHNAKTKPREEQYASTYRVVSVLTVLHQDWLNGKEYKCKVSNKALGAPI |
| | EKTISKAKGQPREPQVYTLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: 5) |
| YTS105HC(k)- | EVQLKESGPGLVQPSQTLSLTCTVSGFSLTSNSVHWVRQPPGKGLEWMGGIWGDGDTDYNSALKSRL |
| TW | SISRDTSKNQVFLKMNSLQTDDTAIYFCTPLIGSWYFDFWGPGTMVTASSASTKGPSVFPLAPSSKS |
| LA.LA.PG.NA | TSGGTAALGCLVKDYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMISRTPEVTCVVVDVSH |
| | EDPEVKFNWYVDGVEVHNAKTKPREEQYASTYRVVSVLTVLHQDWLNGKEYKCKVSNKALGAPIEKT |
| | ISKAKGQPREPQVYTLPPSRDELTKNQVSLWCLVKGFYPSDIAVEWESNGQPENNYKTTPPVLDSDG |
| | SFFLYSKLTVDKSRWQQGNVFSCSVMHEALHNHYTQKSLSLSPGK (SEQ ID NO: 7) |
| YTS105(LAL | EVQLKESGPGLVQPSQTLSLTCTVSGFSLTSNSVHWVRQPPGKGLEWMGGIWGDGDTDYNSALKSRL |
| APGNA)HC | SISRDTSKNQVFLKMNSLQTDDTAIYFCTPLIGSWYFDFWGPGTMVTASSASTKGPSVFPLAPSSKS |
| | TSGGTAALGCLVKDYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMISRTPEVTCVVVDVSH |
| | EDPEVKFNWYVDGVEVHNAKTKPREEQYASTYRVVSVLTVLHQDWLNGKEYKCKVSNKALGAPIEKT |
| | ISKAKGQPREPQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK (SEQ ID NO: 8) |
| HC-YTS105- | EVQLKESGPGLVQPSQTLSLTCTVSGFSLTSNSVHWVRQPPGKGLEWMGGIWGDGDTDYNSALKSRL |
| LALAPGNA- | SISRDTSKNQVFLKMNSLQTDDTAIYFCTPLIGSWYFDFWGPGTMVTASSASTKGPSVFPLAPSSKS |
| hIL2 | TSGGTAALGCLVKDYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGGPSVFLFPPKPKDTLMISRTPEVTCVVVDVSH |
| | EDPEVKFNWYVDGVEVHNAKTKPREEQYASTYRVVSVLTVLHQDWLNGKEYKCKVSNKALGAPIEKT |
| | ISKAKGQPREPQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPGKGGGGSGGGGSGGGGSAPTSSST |
| | KKTQLQLEHLLLDLQMILNGINNYKNPKLTRMLTFKFYMPKKATELKHLQCLEEELKPLEEVLNLAQ |
| | SKNFHLRPRDLISNINVIVLELKGSETTFMCEYADETATIVEFLNRWITFCQSIISTLT (SEQ ID |
| | NO: 9) |
| YTS169- H4A | EVKLQESGGGLVQPGRSLKLSCAASGFNFNDYWMGWVRQAPGKGLEWIGEINKDSSTINYTPSLKDK |
| mlgG2a-IL-2 | FTISRDNAQNTLYLQMSKLGSEDTAIYYCARARGMMVLIIPHYFDYWGQGVMVTVSSAKTTAPSVYP |
| | LAPVCGDTTGSSVTLGCLVKGYFPEPVTLTWNSGSLSSGVHTFPAVLQSDLYTLSSSVTVTSSTWPS |
| | QSITCNVAHPASSTKVDKKIEPRGPTIKPCPPCKCPAPNAAGGPSVFIFPPKIKDVLMISLSPIVTC |
| | VVVDVSEDDPDVQISWFVNNVEVHTAQTQTHREDYASTLRVVSALPIQHQDWMSGKEFKCKVNNKDL |
| | GAPIERTISKPKGSVRAPQVYVLPPPEKEMTKKQVSLTCLVKDFMPEDIYVEWTNNGKTELNYKNTE |
| | PVLKSDGSYFMYSKLTVEKKNWVERNSYSCSVVHEGLHNHHTTKSFSRTPGKGGGGSGGGGSGGGGS |
| | APTSSSTKKTQLQLEHLLLDLQMILNGINNYKNPKLTRMLTFKFYMPKKATELKHLQCLEEELKPLE |
| | EVLNLAQSKNFHLRPRDLISNINVIVLELKGSETTFMCEYADETATIVEFLNRWITFCQSIISTLT |
| | (SEQ ID NO: 11) |
| YTS169 VH- | EVKLQESGGGLVQPGRSLKLSCAASGFNFNDYWMGWVRQAPGKGLEWIGEINKDSSTINYTPSLKDK |
| mlgG2a LALA | FTISRDNAQNTLYLQMSKLGSEDTAIYYCARARGMMVLIIPHYFDYWGQGVMVTVSSAKTTAPSVYP |
| | LAPVCGDTTGSSVTLGCLVKGYFPEPVTLTWNSGSLSSGVHTFPAVLQSDLYTLSSSVTVTSSTWPS |
| | QSITCNVAHPASSTKVDKKIEPRGPTIKPCPPCKCPAPNAAGGPSVFIFPPKIKDVLMISLSPIVTC |
| | VVVDVSEDDPDVQISWFVNNVEVHTAQTQTHREDYNSTLRVVSALPIQHQDWMSGKEFKCKVNNKDL |
| | PAPIERTISKPKGSVRAPQVYVLPPPEEEMTKKQVTLTCMVTDFMPEDIYVEWTNNGKTELNYKNTE |
| | PVLDSDGSYFMYSKLRVEKKNWVERNSYSCSVVHEGLHNHHTTKSFSRTPGK (SEQ ID NO: |
| | 12) |
| hIL2- | APTSSSTKKTQLQLEHLLLDLQMILNGINNYKNPKLTRMLTFKFYMPKKATELKHLQCLEEELKPLE |
| (GGGGS)3 | EVLNLAQSKNFHLRPRDLISNINVIVLELKGSETTFMCEYADETATIVEFLNRWITFCQSIISTLTG |
| YTS169 VH- | GGGSGGGGSGGGGSEVKLQESGGGLVQPGRSLKLSCAASGFNFNDYWMGWVRQAPGKGLEWIGEINK |
| mlgG2a LALA | DSSTINYTPSLKDKFTISRDNAQNTLYLQMSKLGSEDTAIYYCARARGMMVLIIPHYFDYWGQGVMV |
| | TVSSAKTTAPSVYPLAPVCGDTTGSSVTLGCLVKGYFPEPVTLTWNSGSLSSGVHTFPAVLQSDLYT |
| | LSSSVTVTSSTWPSQSITCNVAHPASSTKVDKKIEPRGPTIKPCPPCKCPAPNAAGGPSVFIFPPKI |
| | KDVLMISLSPIVTCVVVDVSEDDPDVQISWFVNNVEVHTAQTQTHREDYNSTLRVVSALPIQHQDWM |
| | SGKEFKCKVNNKDLPAPIERTISKPKGSVRAPQVYVLPPPEEEMTKKQVTLTCMVTDFMPEDIYVEW |
| | TNNGKTELNYKNTEPVLDSDGSYFMYSKLRVEKKNWVERNSYSCSVVHEGLHNHHTTKSFSRTPGK |
| | (SEQ ID NO: 13) |
| hIL2- | APTSSSTKKTQLQLEHLLLDLQMILNGINNYKNPKLTRMLTFKFYMPKKATELKHLQCLEEELKPLE |
| (GGGGS)3 | EVLNLAQSKNFHLRPRDLISNINVIVLELKGSETTFMCEYADETATIVEFLNRWITFCQSIISTLTG |
| YTS169 VH-k- | GGGSGGGGSGGGGSEVKLQESGGGLVQPGRSLKLSCAASGFNFNDYWMGWVRQAPGKGLEWIGEINK |
| mlgG2a | DSSTINYTPSLKDKFTISRDNAQNTLYLQMSKLGSEDTAIYYCARARGMMVLIIPHYFDYWGQGVMV |
| LALAPGNA | TVSSAKTTAPSVYPLAPVCGDTTGSSVTLGCLVKGYFPEPVTLTWNSGSLSSGVHTFPAVLQSDLYT |
| | LSSSVTVTSSTWPSQSITCNVAHPASSTKVDKKIEPRGPTIKPCPPCKCPAPNAAGGPSVFIFPPKI |
| | KDVLMISLSPIVTCVVVDVSEDDPDVQISWFVNNVEVHTAQTQTHREDYASTLRVVSALPIQHQDWM |
| | SGKEFKCKVNNKDLGAPIERTISKPKGSVRAPQVYVLPPPEEEMTKKQVTLWCMVTDFMPEDIYVEW |
| | TNNGKTELNYKNTEPVLDSDGSYFMYSKLRVEKKNWVERNSYSCSVVHEGLHNHHTTKSFSRTPGK |
| | (SEQ ID NO: 14) |
| YTS169-H4B- | EVKLQESGGGLVQPGRSLKLSCAASGFNFNDYWMGWVRQAPGKGLEWIGEINKDSSTINYTPSLKDK |
| mlgG2a | FTISRDNAQNTLYLQMSKLGSEDTAIYYCARARGMMVLIIPHYFDYWGQGVMVTVSSAKTTAPSVYP |
| | LAPVCGDTTGSSVTLGCLVKGYFPEPVTLTWNSGSLSSGVHTFPAVLQSDLYTLSSSVTVTSSTWPS |
| | QSITCNVAHPASSTKVDKKIEPRGPTIKPCPPCKCPAPNAAGGPSVFIFPPKIKDVLMISLSPIVTC |
| | VVVDVSEDDPDVQISWFVNNVEVHTAQTQTHREDYASTLRVVSALPIQHQDWMSGKEFKCKVNNKDL |
| | GAPIERTISKPKGSVRAPQVYVLPPPEEEMTKKQVSLTCLVKDFMPEDIYVEWTNNGKTELNYKNTE |
| | PVLDSDGSYFMYSELTVEKKNWVERNSYSCSVVHEGLHNHHTTDSFSRTPGK (SEQ ID NO: 15) |
| YTS169 VH-h- | EVKLQESGGGLVQPGRSLKLSCAASGFNFNDYWMGWVRQAPGKGLEWIGEINKDSSTINYTPSLKDK |
| mlgG2a | FTISRDNAQNTLYLQMSKLGSEDTAIYYCARARGMMVLIIPHYFDYWGQGVMVTVSSAKTTAPSVYP |
| LALAPGNA | LAPVCGDTTGSSVTLGCLVKGYFPEPVTLTWNSGSLSSGVHTFPAVLQSDLYTLSSSVTVTSSTWPS |
| | QSITCNVAHPASSTKVDKKIEPRGPTIKPCPPCKCPAPNAAGGPSVFIFPPKIKDVLMISLSPIVTC |
| | VVVDVSEDDPDVQISWFVNNVEVHTAQTQTHREDYASTLRVVSALPIQHQDWMSGKEFKCKVNNKDL |
| | GAPIERTISKPKGSVRAPQVYVLPPPEEEMTKKQVTLACAVTDFMPEDIYVEWTNNGKTELNYKNTE |
| | PVLDSDGSYFMVSKLRVEKKNWVERNSYSCSVVHEGLHNHHTTKSFSRTPGK (SEQ ID NO: 16) |
| HC(k)- | EVQLVQSGAEVKKPGATVKISCKASGYTFTDFTMHWVQQAPGKGLEWIGLVNPNNGGNIYAEKFQGR |
| 02(VH6)TW- | VTLTVDTSTSTAYMELSSLRSEDTAVYYCGRVPLYTSHGMDYWGQGTLVTVSSASTKGPSVFPLAPS |
| (IL2) | SKSTSGGTAALGCLVKDYFPEPVTVSWNSGALTSGVHTFPAVLQSSGLYSLSSVVTVPSSSLGTQTY |
| | ICNVNHKPSNTKVDKKVEPKSCDKTHTCPPCPAPEAAGGPSVFLFPPKPKDTLMISRTPEVTCVVVD |
| | VSHEDPEVKFNWYVDGVEVHNAKTKPREEQYASTYRVVSVLTVLHQDWLNGKEYKCKVSNKALGAPI |
| | EKTISKAKGQPREPQVYTLPPSRDELTKNQVSLWCLVKGFYPSDIAVEWESNGQPENNYKTTPPVLD |
| | SDGSFFLYSKLTVDKSRWQQGNVFSCSVMHEALHNHYTQKSLSLSPGKGGGSGGGGSGGGGSGGGGS |
| | APTSSSTKKTQLQLEHLLLDLQMILNGINNYKNPKLTRMLTFKFYMPKKATELKHLQCLEEELKPLE |
| | EVLNLAQSKNFHLRPRDLISNINVIVLELKGSETTFMCEYADETATIVEFLNRWITFCQSIISTLT |
| | (SEQ ID NO: 19) |
| HC(h)- | EVQLVQSGAEVKKPGATVKISCKASGYTFTDFTMHWVQQAPGKGLEWIGLVNPNNGGNIYAEKFQGR |
| 02(VH6)- | VTLTVDTSTSTAYMELSSLRSEDTAVYYCGRVPLYTSHGMDYWGQGTLVTVSSASTKGPSVFPLAPS |
| TS.LA.YV.LA. | SKSTSGGTAALGCLVKDYFPEPVTVSWNSGALTSGVHTFPAVLQSSGLYSLSSVVTVPSSSLGTQTY |
| LA.PG.NA | ICNVNHKPSNTKVDKKVEPKSCDKTHTCPPCPAPEAAGGPSVFLFPPKPKDTLMISRTPEVTCVVVD |
| | VSHEDPEVKFNWYVDGVEVHNAKTKPREEQYASTYRVVSVLTVLHQDWLNGKEYKCKVSNKALGAPI |
| | EKTISKAKGQPREPQVYTLPPSRDELTKNQVSLSCAVKGFYPSDIAVEWESNGQPENNYKTTPPVLD |
| | SDGSFFLVSKLTVDKSRWQQGNVFSCSVMHEALHNHYTQKSLSLSPGK* (SEQ ID NO: 21) |

TABLE 8
Exemplary Light Chains

| Light Chain | |
|---|---|
| protein | Amino Acid Sequence |
| YTS105 Light | DIVMTQSPSSLAVSAGERVTLNCKASQNVRNNIAWYQQKPGQSPKLLIYYASYRYTGVPDRFTGDGF |
| Chain | GTDFTLAINSVQADDAAFYYCQRIYNSPYTFGAGTKLELIRTVAAPSVFIFPPSDEQLKSGTASVVC |
| | LLNNFYPREAKVQWKVDNALQSGNSQESVTEQDSKDSTYSLSSTLTLSKADYEKHKVYACEVTHQGL |
| | SSPVTKSFNRGEC (SEQ ID NO: 2) |
| YTS169 Light | DIVLTQSPAMAMSPGERITISCRASESVSTRMHWYQQKPGQQPKLLIYGASNLESGVPARFSGSGSG |
| Chain | TDFTLTIDPVEANDTATYFCQQSWYDPWTFGGGTKLELKRADAAPTVSIFPPSSEQLTSGGASVVCF |
| | LNNFYPKDINVKWKIDGSERQNGVLNSWTDQDSKDSTYSMSSTLTLTKDEYERHNSYTCEATHKTST |
| | SPIVKSFNRNEC (SEQ ID NO: 4) |
| Humanized | EIVLTQSPATLSLSPGERATISCRASESVDSYDINSMHWYQQKPGQAPRLLIYRASHLQSGIPARFS |
| 02A01-VK3- | GSGSRTDFTLTISSLEPEDFATYYCQQSNEDPLTFGQGTKLEIKRTVAAPSVFIFPPSDEQLKSGTA |
| Full IgG Light | SVVCLLNNFYPREAKVQWKVDNALQSGNSQESVTEQDSKDSTYSLSSTLTLSKADYEKHKVYACEVT |
| Chain | HQGLSSPVTKSFNRGEC (SEQ ID NO: 6) |
| YTS105-IL2 | DIVMTQSPSSLAVSAGERVTLNCKASQNVRNNIAWYQQKPGQSPKLLIYYASYRYTGVPDRFTGDGF |
| LC | GTDFTLAINSVQADDAAFYYCQRIYNSPYTFGAGTKLELIRTVAAPSVFIFPPSDEQLKSGTASVVC |
| | LLNNFYPREAKVQWKVDNALQSGNSQESVTEQDSKDSTYSLSSTLTLSKADYEKHKVYACEVTHQGL |
| | SSPVTKSFNRGECGGGGSGGGGSGGGGSAPTSSSTKKTQLQLEHLLLDLQMILNGINNYKNPKLTRM |
| | LTFKFYMPKKATELKHLQCLEEELKPLEEVLNLAQSKNFHLRPRDLISNINVIVLELKGSETTFMCE |
| | YADETATIVEFLNRWITFCQSIISTLT (SEQ ID NO: 10) |
| LC-YTS169- | DIVLTQSPAMAMSPGERITISCRASESVSTRMHWYQQKPGQQPKLLIYGASNLESGVPARFSGSGSG |
| mK-hIL2 | TDFTLTIDPVEANDTATYFCQQSWYDPWTFGGGTKLELKRADAAPTVSIFPPSSEQLTSGGASVVCF |
| | LNNFYPKDINVKWKIDGSERQNGVLNSWTDQDSKDSTYSMSSTLTLTKDEYERHNSYTCEATHKTST |
| | SPIVKSFNRNECGGGGSGGGGSGGGGSAPTSSSTKKTQLQLEHLLLDLQMILNGINNYKNPKLTRML |
| | TFKFYMPKKATELKHLQCLEEELKPLEEVLNLAQSKNFHLRPRDLISNINVIVLELKGSETTFMCEY |
| | ADETATIVEFLNRWITFCQSIISTLT* (SEQ ID NO: 17) |
| hIL2- | APTSSSTKKTQLQLEHLLLDLQMILNGINNYKNPKLTRMLTFKFYMPKKATELKHLQCLEEELKPLE |
| (GGGGS)3- | EVLNLAQSKNFHLRPRDLISNINVIVLELKGSETTFMCEYADETATIVEFLNRWITFCQSIISTLTG |
| YTS169-VL- | GGGSGGGGSGGGGSDIVLTQSPAMAMSPGERITISCRASESVSTRMHWYQQKPGQQPKLLIYGASNL |
| mKappa | ESGVPARFSGSGSGTDFTLTIDPVEANDTATYFCQQSWYDPWTFGGGTKLELKRADAAPTVSIFPPS |
| | SEQLTSGGASVVCFLNNFYPKDINVKWKIDGSERQNGVLNSWTDQDSKDSTYSMSSTLTLTKDEYER |
| | HNSYTCEATHKTSTSPIVKSFNRNEC* (SEQ ID NO: 18) |
| LC-02-VK3- | EIVLTQSPATLSLSPGERATISCRASESVDSYDINSMHWYQQKPGQAPRLLIYRASHLQSGIPARFS |
| IL2 | GSGSRTDFTLTISSLEPEDFATYYCQQSNEDPLTFGQGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVYACEVT |
| | HQGLSSPVTKSFNRGECGGGGSGGGGSGGGGSGGGGSAPTSSSTKKTQLQLEHLLLDLQMILNGINN |
| | YKNPKLTRMLTFKFYMPKKATELKHLQCLEEELKPLEEVLNLAQSKNFHLRPRDLISNINVIVLELK |
| | GSETTFMCEYADETATIVEFLNRWITFCQSIISTLT* (SEQ ID NO: 20) |

TABLE 9
Exemplary Fusion Proteins

| Protein Name | Polypeptide Components |
|---|---|
| mCD8 Fab | YTS105HC (SEQ ID NO: 1) |
| IL2 (LC) | YTS105-IL2 LC (SEQ ID NO: 10) |
| (YTS105) | |
| mCD8 Fab Fc | YTS105HC (k)-TW LA.LA.PG.NA (SEQ ID NO: 7) |
| IL2 (LC) | HC(h)-TS.LA.YV.LA.LA.PG.NA (SEQ ID NO: 125) |
| (YTS105) | YTS105-IL2 LC (SEQ ID NO: 10) |
| mCD8 IgG | YTS105(LALAPGNA)HC (SEQ ID NO: 8) |
| IL2 (LC) | YTS105-IL2 LC (SEQ ID NO: 10) |
| (YTS105) | |
| mCD8 IgG | HC-YTS105-LALAPGNA-hIL2 (SEQ ID NO: 9) |
| IL2 (HC) | YTS105 Light Chain (SEQ ID NO: 2) |
| (YTS105) | |
| mCD8 IgG | VH-YTS169-2aLALAPGNA (SEQ ID NO: 3) |
| IL2 (LC) | LC-YTS169-mK-hIL2 (SEQ ID NO: 17) |
| (YTS169) | |
| mCD8 IgG IL2 | YTS169-H4A mIgG2a-IL-2 (SEQ ID NO: 11) |
| 1x (HC) | YTS169-H4B-mIgG2a (SEQ ID NO: 15) |
| (YTS169) | YTS169-VL-mKappa (SEQ ID NO: 4) |
| mCD8 IgG | YTS169 VH-mIgG2a LALA (SEQ ID NO: 12) |
| IL2 (LN) | hIL2-(GGGGS)3-YTS169-VL-mKappa (SEQ ID NO: 18) |
| (YTS169) | |
| mCD8 IgG | hIL2-(GGGGS)3 YTS169 VH-mIgG2a LALA (SEQ ID NO: 13) |
| IL2 (HN) | YTS169-VL-mKappa (SEQ ID NO: 4) |
| (YTS169) | |
| mCD8 IgG IL2 | hIL2-(GGGGS)3 YTS169 VH-k-mIgG2a LALAPGNA (SEQ ID NO: 14) |
| 1x (HN) | YTS169 VH-h-mIgG2a LALAPGNA (SEQ ID NO: 16) |
| (YTS169) | YTS169-VL-mKappa (SEQ ID NO: 4) |
| hCD8 IgG IL 2 | VH6 Heavy Chain (SEQ ID NO: 5) |
| (LC) (VH6/VK3) | LC-02-VK3-IL2 (SEQ ID NO: 20) |
| hCD8 IgG IL2 | HC(k)-02(VH6)TW-(IL2) (SEQ ID NO: 19) |
| 1x (HC) | HC(h)-02(VH6)-TS.LA.YV.LA.LA.PG.NA (SEQ ID NO: 21) |
| (VH6/VK3) | LC-02-VK3 277576 (SEQ ID NO: 6) |
| FIG. 2A | HC: HC-02(VH6)(02A01 VH6, human CH1) (SEQ ID NO: 111) |
| Fab IL2 (LC) | LC: LC-02-VK3-LM-IL2 (D30E, N35Q) |
| | (SEQ ID NO: 112) |
| FIG. 2B | HC1: HC(k)-02(VH6)TW.LA.LA.NA |
| Fab-Fc IL2 (LC) | (02A01 VH6, human CH1, CH2-CH3) (SEQ ID NO: 113) |
| | HC2: HC (h)-CH2-CH3-TS.LA.YV.LA.LA.NA (human CH2-CH3) (SEQ |
| | ID NO: 114) |
| | LC: LC-02-VK3-LM-IL2(D30E, N35Q) (SEQ ID NO: 112) |
| FIG. 2C | HC: HC-02(VH6)LA.LA.NA |
| IgG IL2 (LC) | (02A01 VH6, human CH1, CH2-CH3) (SEQ ID NO: 115) |
| | LC: LC-02-VK3-LM-IL2(D30E, N35Q) (SEQ ID NO: 112) |
| FIG. 2D | HC: HC-02(VH6)LA.LA.NA-IL2 |
| IgG IL2 (HC) | (02A01 VH6, human CH1, CH2-CH3-IL2) (SEQ ID NO: 116) |
| | LC: LC-02-VK3-LM |
| | (SEQ ID NO: 117) |
| FIG. 2E | HC1: HC(k)-02(VH6)TW.LA.LA.NA-IL2 |
| IgG IL2 1X (HC) | (02A01 VH6, human CH1, CH2-CH3-IL2) (SEQ ID NO: 118) |
| | HC2: HC(h)-02(VH6)TS LA.YV.LA.LA.NA |
| | (02A01 VH6, human CH1, CH2-CH3) (SEQ ID NO: 119) |
| | LC: LC-02-VK3-LM (SEQ ID NO: 117) |
| FIG. 2F | HC: IL2-HC-02(VH6)LA.LA.NA |
| IgG IL2 (HN) | (IL2-02A01 VH6, human CH1, CH2-CH3) (SEQ ID NO: 120) |
| | LC: LC-02-VK3-LM (SEQ ID NO: 117) |
| FIG. 2G | HC: HC-02(VH6)LA.LA.NA |
| IgG IL2 (LN) | (02A01 VH6, human CH1, CH2-CH3) (SEQ ID NO: 115) |
| | LC: IL2-LC-02-VK3-LM (D30E, N35Q) (SEQ ID NO: 121) |
| FIG. 2H | HC1: HC(k)-02(VH6)TW.LA.LA.NA |
| IgG IL2 1X (HN) | (02A01 VH6, human CH1, CH2-CH3) (SEQ ID NO: 113) |
| | HC2: IL2-HC(h)-02(VH6)TS.LA.YV.LA.LA.NA |
| | (IL2-02A01 VH6, human CH1, CH2-CH3) (SEQ ID NO: 122) |
| | LC: LC-02-VK3-LM (SEQ ID NO: 117) |
| FIG. 2I | HC: HC-02(VH6)-IL2 (02A01 VH6, human CH1, IL2) (SEQ ID |
| Fab IL2 (HC) | NO: 123) |
| | LC: LC-02-VK3-LM (SEQ ID NO: 117) |
| FIG. 2J | HC1: HC(k)-02(VH6)TW.LA.LA.NA-IL2 |
| Fab-Fc | (02A01 VH6, human CH1, CH2-CH3-IL2) (SEQ ID NO: 118) |
| IL2 (HC) (at | HC2: HC(h)-CH2-CH3-TS.LA.YV.LA.LA.NA |
| Knob chain) | (human CH2-CH3) (SEQ ID NO: 114) |
| | LC: LC-02-VK3-LM (SEQ ID NO: 117) |
| FIG. 2K | HC1: HC(k)-02(VH6)TW.LA.LA.NA |
| Fab-Fc | (02A01 VH6, human CH1, CH2-CH3) (SEQ ID NO: 113) |
| IL2 (HC) (at | HC2: HC(h)-CH2-CH3-TS.LA.YV.LA.LA.NA-IL2 |
| Hole chain) | (human CH2-CH3-IL2) (SEQ ID NO: 124) |
| | LC: LC-02-VK3-LM (SEQ ID NO: 117) |

### III. Methods of Treatment

In some aspects, provided herein is a method of preventing or treating a disease or condition in a subject in need thereof. In some aspects, the method comprises administering to the subject any of the fusion proteins described herein. In some aspects, the method comprises administering to the subject any of the nucleic acids described herein. In some aspects, the method comprises administering to the subject any of the vectors described herein. In some aspects, the method comprises administering to the subject any of the recombinant cells described herein.

In some aspects, the disease or condition is a cancer or an infection.

In some aspects, the cancer is a bladder cancer, breast cancer, uterine cancer, endometrial carcinoma, ovarian cancer, colorectal cancer, colon cancer, head and neck cancer, lung cancer, stomach cancer, germ cell cancer, bone cancer, squamous cell cancer, skin cancer, neoplasm of the central nervous system, lymphoma, leukemia, sarcoma, virus-related cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, myeloma, salivary gland carcinoma, kidney cancer, basal cell carcinoma, melanoma, prostate cancer, vulval cancer, thyroid cancer, testicular cancer, esophageal cancer, or head or neck cancer, and any combinations thereof.

In some aspects, provided herein is a method of killing a cancer cell, the method comprising contacting the cancer cell and a CD8+ T cell to any of the fusion proteins disclosed herein. In some aspects, the cancer cell is a bladder cancer cell, breast cancer cell, uterine cancer cell, endometrial carcinoma cell, ovarian cancer cell, colorectal cancer cell, colon cancer cell, head and neck cancer cell, lung cancer cell, stomach cancer cell, germ cell cancer cell, bone cancer cell, squamous cell cancer cell, skin cancer cell, neoplasm cell of the central nervous system, lymphoma cell, leukemia cell, sarcoma cell, virus-related cancer cell, small-cell lung cancer cell, non-small cell lung cancer cell, gastrointestinal cancer cell, Hodgkin's or non-Hodgkin's lymphoma cell, pancreatic cancer cell, glioblastoma cell, glioma cell, cervical cancer cell, liver cancer cell, myeloma cell, salivary gland carcinoma cell, kidney cancer cell, basal cell carcinoma cell, melanoma cell, prostate cancer cell, vulval cancer cell, thyroid cancer cell, testicular cancer cell, esophageal cancer cell, and any combinations thereof.

In some aspects, the method further comprises administering an anti-cancer agent.

In some aspects, the anti-cancer agent comprises an immunomodulator, an adoptive cell therapy, a checkpoint inhibitor, a small molecule based anti-cancer therapy, an antibody based cancer therapy, a cancer vaccine, or a chemotherapeutic agent. In some aspects, the immunomodulator is an immunostimulator. In some aspects, the immunostimulator comprises an immunostimulatory cytokine, an agonist of a costimulatory molecule, and/or an inhibitor of an immune checkpoint protein.

In some aspects, the immunostimulatory cytokine is selected from the group consisting of IL-12, IL-15, IL-2, IL-6, IL-7, IL-18, IL-21, IL-23, and IL-27, and any combinations thereof.

In some aspects, the immunomodulator comprises an immunosuppressor. In some aspects, the immunosuppressor comprises an immunosuppressive cytokine, an inhibitor of a costimulatory molecule, and/or an agonist of an immune checkpoint protein.

In some aspects, the checkpoint inhibitor is selected from the group consisting of a PD-1 antagonist, a PD-L1 antagonist, a LAG-3 antagonist, a TIM-3 antagonist, a CTLA-4 antagonist, a B7-H3 antagonist, a B7-H4 antagonist, a BTLA antagonist, a KIR antagonist, a VISTA antagonist, a TIGIT antagonist, an IDO antagonist, a Siglec-15 antagonist, a GAL-9 antagonist, an HVEM antagonist, or combinations thereof.

### IV. Combination Therapies

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist, TIGIT antagonist, IDO antagonist, Siglec-15 antagonist, GAL-9 antagonist, or an HVEM antagonist. In some aspects, the checkpoint inhibitor is a PD-1 antagonist.

In some aspects, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described, e.g., in U.S. Pat. Nos. 8,728,474 and 9,073,994, and EP Patent No. 1537878B1, and can include, e.g., the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

In some aspects, a composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO1998042752, WO2000037504, and WO2001014424, and European Patent No. EP 1212422 B1. Exemplary CTLA-4 antibodies include ipilimumab or tremelimumab.

In some aspects, a composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an agent selected from an ALK inhibitor, an ATR inhibitor, an A2A Antagonist, a Base Excision Repair inhibitor, a Bcr-Abl Tyrosine Kinase inhibitor, a Bruton's Tyrosine Kinase inhibitor, a CDC7 inhibitor, a CHK1 inhibitor, a Cyclin-Dependent Kinase inhibitor, a DNA-PK inhibitor, an inhibitor of both DNA-PK and mTOR, a DNMT1 inhibitor, a DNMT1 inhibitor plus 2-chloro-deoxyadenosine, an HDAC inhibitor, a Hedgehog Signaling Pathway inhibitor, an IDO inhibitor, a JAK inhibitor, a mTOR inhibitor, a MEK inhibitor, a MELK inhibitor, a MTH1 inhibitor, a PARP inhibitor, a Phosphoinositide 3-Kinase inhibitor, an inhibitor of both PARP1 and DHODH, a Proteasome inhibitor, a Topoisomerase-II inhibitor, a Tyrosine Kinase inhibitor, a VEGFR inhibitor, and a WEE1 inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

The fusion proteins (e.g., CD8 antibody-IL2 fusion proteins) disclosed herein may also be used in combination with an immune cell therapy (e.g., adoptive cell therapy). In some aspects, a method of treatment disclosed herein further comprises administering to the subject an immune cell composition comprising immune cells (e.g., T cells, B cells, NK cells, tumor-infiltrating lymphocytes, and/or dendritic cells). In some aspects, the immune cell composition comprises CD8+ T cells. In some aspects, at least 40%, at least 50%, at least 60%, at least 70%, at least 80%, at least 90%, at least 95%, or at least 99% of the cells in the immune cell composition are CD8+ T cells. In some aspects, the immune cell composition further comprises CD4+ T cells, optionally wherein at least 20%, at least 30%, at least 40%, or at least 50% of the cells in the immune cell composition are CD4+ T cells.

Immune cells can be obtained from a blood sample of a patient by apheresis. In some aspects, a lymphocyte-rich fraction and a monocyte-rich fraction can be acquired by elutriating peripheral blood mononuclear cells (PBMCs) of the patient. The monocyte-rich fraction can then be used to prepare antigen-presenting cells (APCs) for priming T cells, which can be obtained from the lymphocyte-rich fraction. Exemplary methods are described in WO2020055931 and U.S. Pat. Nos. 9,963,677 and 9,642,906. In some aspects, the immune cell therapy is autologous, i.e., immune cells obtained from a patient, after in vitro culture, are administered to the same patient. In some aspects, the immune cell therapy is allogeneic, optionally wherein the immune cells are genetically engineered to inactivate a component of class I MHC (e.g., 02M).

In some aspects, the combination therapy of the fusion protein (e.g., immunostimulatory fusion protein) and the immune cell composition has a partially additive effect, wholly additive effect, or greater than additive effect (e.g., synergistic effect). In some aspects, the fusion protein improves the efficacy of the immune cell composition (e.g., leads to greater cancer remission, greater reduction in relapse, and/or greater reduction in symptoms). In some aspects, the combination therapy permits use of a lower dose of the immune cell composition compared to the dose of normally required to achieve similar effects when administered as a monotherapy.

In some aspects, the fusion protein and the immune cell composition are administered simultaneously. For example, in some aspects, administration of the fusion protein is continuous or intermittent during the course of the immune cell therapy. In some aspects, the fusion protein and the immune cell composition are administered in a single composition, optionally wherein the fusion protein binds CD8+ T cells in the immune cell composition through interaction with CD8. Also provided herein is a composition comprising the fusion protein and the immune cell composition.

In some aspects, the fusion protein and the immune cell composition are administered sequentially (with or without overlap). In some aspects, the fusion protein is administered after the administration of a dose of the immune cell composition has completed. In some aspects, the fusion protein is administered at least one week, at least two weeks, at least three weeks, at least four weeks, at least one month, at least two months, at least three months, at least four months, at least five months, at least six months, at least seven months, at least eight months, at least nine months, at least ten months, at least 11 months, at least 12 months, at least one year, or at least two years after the administration of the immune cell composition.

In some aspects, the fusion protein is administered in multiple doses. In some aspects s, at least one dose of the fusion protein is administered at least one week, at least two weeks, at least three weeks, at least four weeks, at least one month, at least two months, at least three months, at least four months, at least five months, at least six months, at least seven months, at least eight months, at least nine months, at least ten months, at least 11 months, at least 12 months, at least one year, or at least two years after the administration of the immune cell composition. In some aspects, at least one dose of the fusion protein is administered simultaneously (e.g., in a single composition) with the immune cell therapy, and at least one other dose of the fusion protein is administered at least one week, at least two weeks, at least three weeks, at least four weeks, at least one month, at least two months, at least three months, at least four months, at least five months, at least six months, at least seven months, at least eight months, at least nine months, at least ten months, at least 11 months, at least 12 months, at least one year, or at least two years after the administration of the immune cell composition. Without wishing to be bound by theory, it is contemplated that the later dose can function as a booster dose to augment the survival, proliferation, memory formation, and/or function of the earlier-administered immune cell composition or cells derived therefrom in vivo.

Immune cells (e.g., T cells, e.g., CD8+ T cells) can have antigen specificity via a receptor expressed on the cell surface. T cells can naturally express T cell receptors (TCRs), such as αβTCRs or γδTCRs, and can be “trained” to express TCRs that target a given antigen or epitope. Immune cells can also be genetically engineered to express a recombinant TCR or a chimeric antigen receptor (CAR). Where the combination therapy disclosed herein is used to treat cancer, the TCR or CAR can target one or more tumor-associated antigens or epitopes thereof.

In some aspects, the T cells have been trained to recognize one or more T cell epitopes of an antigen (e.g., tumor-associated antigen). In some aspects, the T cells have been primed by APCs, for example, by co-culturing a T cell composition (e.g., the lymphocyte-rich fraction of PBMCs) with APCs that present one or more T cell epitopes of the antigen. Such co-culture, optionally carried out at a ratio of about 1:1 to about 40:1 (T cells to APCs), can result in expansion of T cells that are reactive to the one or more T cell epitopes. Cytokines such as IL-2, IL-6, IL-7, IL-12, IL-15, and/or IL-21 can be added to the cell culture to increase survival, proliferation, and/or memory formation of the T cells. In some aspects, the APCs present a plurality of T cell epitopes, optionally from a plurality of antigens (e.g., tumor-associated antigens), thereby to produce multi-targeted T cells (MTCs).

In some aspects, the immune cell composition comprises immune cells (e.g., T cells, e.g., CD8+ T cells) engineered to express an antigen receptor. Exemplary antigen receptors include TCRs and CARs. In some aspects, the immune cells are genetically engineered to inactivate their endogenous TCRs, for example, by knocking out the TRAC or TRBC gene, to reduce ligand-independent tonic T cell signaling and enhances T cell potency.

In some aspects, the immune cells express a CAR comprising an extracellular antigen-binding domain, a transmembrane domain, and a primary signaling domain comprising a functional intracellular signaling domain derived from a stimulatory molecule. In some aspects, the CAR further comprises one or more costimulatory signaling domains comprising functional signaling domains derived from one or more costimulatory molecules. Further examples of CAR are provided in U.S. Pat. Nos. 7,446,190 and 9,181,527, U.S. Patent Application Publication Nos. 2016/0340406 and 2017/0049819, and International Patent Application Publication No. WO2018/140725.

In some aspects, the extracellular antigen-binding domain of the CAR comprises an antigen-binding site, as defined herein, that binds a target antigen. In some aspects, the extracellular antigen-binding domain comprises a Fab fragment or an scFv. In some aspects, the extracellular antigen-binding domain comprises an scFv comprising a heavy chain variable domain and a light chain variable domain linked by a peptide linker (e.g., a peptide linker described in the “Linkers” subsection above. In some aspects, the extracellular antigen-binding domain comprises an antigen (e.g., autoimmune antigen), wherein cells reactive to this antigen (e.g., expressing antibodies that bind the antigen) are target cells of the immune cell therapy disclosed herein.

The extracellular antigen-binding domain can be fused to the transmembrane domain of the CAR. In some aspects, the transmembrane domain of the CAR is derived from a naturally occurring transmembrane protein. In certain embodiments, the transmembrane domain is capable of signaling to the intracellular domain(s) whenever the CAR has bound to a target. In some aspects, the transmembrane domain comprises the transmembrane region(s) of one or more proteins selected from the group consisting of TCR α chain, TCR β chain, TCR ζ chain, CD28, CD3F, CD45, CD4, CD5, CD8, CD9, CD16, CD22, EGFR, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11 d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C. In some aspects, the transmembrane domain is one that naturally is associated with one of the domains (e.g., primary signaling domain or co-stimulatory signaling domain) in the CAR. In some aspects, the transmembrane domain can be selected or modified by amino acid substitution to avoid multimerization with a transmembrane domain of the same or a different surface membrane protein, thereby to minimize interactions with other members of a receptor complex (e.g., the CAR complex). In some aspects, the transmembrane domain is capable of homodimerization with another CAR on the immune cell (e.g., T cell) surface. In another embodiment, the amino acid sequence of the transmembrane domain may be modified or substituted so as to minimize interactions with the binding domains of the native binding partner present in the same immune cell.

The extracellular antigen-binding domain of the CAR can be connected to the transmembrane domain by a hinge region. A variety of hinges can be employed, including but not limited to the human Ig (immunoglobulin) hinge (e.g., an IgG4 hinge, an IgD hinge), a Gly-Ser linker, a (G4S)4 linker, a KIR2DS2 hinge, and a CD8α hinge.

The intracellular signaling domain of the CAR comprises a primary signaling domain (i.e., a functional signaling domain derived from a stimulatory molecule) and, optionally, one or more costimulatory signaling domains (i.e., functional signaling domains derived from at least one costimulatory molecule). These intracellular signaling domains are responsible for an immune cell response, including but not limited to proliferation, differentiation, and activation of a specialized function of the immune cell (e.g., cytotoxic activity or secretion of cytokines of a T cell) in which the CAR has been placed in. The intracellular signaling domain is thus meant to include any truncated portion of the intracellular signaling domain sufficient to transduce the effector function signal.

Primary signaling domains that act in a stimulatory manner may contain signaling motifs which are known as immunoreceptor tyrosine-based activation motifs or ITAMs. Examples of ITAM containing cytoplasmic signaling sequences that are of particular use in the present application include those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In some aspects, the primary signaling domain comprises a functional, cytoplasmic signaling domain derived from CD3 zeta, FcR gamma, FcR beta, CD3 gamma, CD3 delta, CD3 epsilon, CD5, CD22, CD79a, CD79b, CD66d, 4-1BB, common FcR gamma (FCER1G), Fc gamma RIIa, FcR beta (Fc Epsilon Rib), DAP10, and/or DAP12.

A costimulatory signaling domains comprises a functional signaling domain derived from a costimulatory molecule, a cell surface molecule other than an antigen receptor or its ligands that is required for an efficient response of lymphocytes to an antigen. Examples of costimulatory molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, CD83 ligands, CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, and PAG/Cbp. In some aspects, a costimulatory signaling domain of the CAR comprises a functional signaling domain of a costimulatory molecule described herein, e.g., OX40, CD27, CD28, CD30, CD40, PD-1, CD2, CD7, CD258, NKG2C, B7-H3, a CD83 ligand, ICAM-1, LFA-1 (CD11a/CD18), ICOS and 4-1BB (CD137), or any combination thereof.

The signaling domains within the cytoplasmic portion of the CAR may be linked to each other in a random or specified order. In some aspects, a costimulatory signaling domain is deployed N-terminal to the primary signaling domain. Optionally, the signaling domains are linked by a short oligo- or polypeptide linker, for example, between 2 and 10 amino acids in length.

In some aspects, the immune cells of the immune cell composition express a recombinant TCR. Generally, an αβTCR is capable of binding a peptide presented by a major histocompatibility complex (MHC) molecule, whereas a γδTCR does not require MHC-mediated antigen presentation. In some aspects, the TCR comprises variable α and β chains (also known as TCRα and TCRβ, respectively) or variable γ and δ chains (also known as TCRγ and TCRδ, respectively), or a portion thereof that binds the antigen (e.g., peptide-MHC complex) comprising the α and β chain variable domains or the γ and δ chain variable domains. Generally, the variable domains of a TCR comprise complementarity determining regions involved in recognition of the antigen (e.g., peptide-MHC complex). In certain embodiments, the TCR expressed by the immune cells is cloned from a naturally occurring T cell.

In some aspects, the TCR comprises a constant domain, a transmembrane domain, and/or a short cytoplasmic tail. In some aspects, each chain of the TCR can possess one N-terminal immunoglobulin variable domain, one immunoglobulin constant domain, a transmembrane region, and a short cytoplasmic tail at the C-terminal end. In some aspects, the TCR is associated with invariant proteins of the CD3 complex involved in mediating signal transduction.

The immune cells disclosed herein can be genetically engineered to express a CAR or TCR by introducing a nucleic acid encoding the CAR or TCR. In some aspects, the nucleic acid is a DNA molecule (e.g., a cDNA molecule). In some aspects, the nucleic acid further comprises an expression control sequence (e.g., promoter and/or enhancer) operably linked to the CAR or TCR coding sequence. In some aspects, the immune cells are transduced by a vector, such as a viral vector (e.g., AAV vector, lentiviral vector, or adenoviral vector) or a non-viral vector (e.g., plasmid), comprising a nucleic acid encoding the CAR or TCR. In some aspects, the nucleic acid is an RNA molecule (e.g., an mRNA molecule). Methods for generating and modifying mRNA for use in transfection are disclosed in U.S. Pat. Nos. 8,278,036; 8,883,506, and 8,716,465. In some aspects, the nucleic acid encodes an amino acid sequence comprising a signal peptide at the N-terminus of the CAR or TCR. Such signal peptide can facilitate cell surface localization of the CAR or TCR when it is expressed in an effector cell, and is cleaved from the CAR during cellular processing.

In some aspects, the immune cell composition comprises a protein cluster, also known as a protein nanogel, comprising a plurality of therapeutic protein monomers reversibly crossed-linked to one another by biodegradable linkers. The size of the protein nanogel can range from 30 nm to 1,000 nm in diameter, as measured by dynamic light scattering. In some aspects, the protein nanogel can be prepared by reacting the plurality of therapeutic protein monomers with a plurality of cross-linkers. Examples of therapeutic protein monomers comprised in a protein nanogel include, without limitation, cytokines, chemokines, enzymes, co-factors, ligands, receptors and soluble fragments thereof, and other regulatory factors, antibodies and fragments thereof (e.g., IgG, Fab, scFv, single domain antibodies), and engineered proteins such as antibody Fc domain or human serum albumin fused with a regulatory factor or antibody or fragment thereof described above. Additional methods of reversibly cross-linking protein monomers to form a protein nanogel are disclosed in WO2015048498, WO2017218533, WO2019050978, WO2019050977, and WO2020205808.

It is understood that the fusion proteins disclosed herein may, under certain circumstances, cause side effects such as neurotoxicity under certain conditions. Accordingly, in some aspects, the second therapeutic agent that can be used in combination with the fusion protein comprises an agent that mitigates a side effect of the fusion protein, e.g., reduces neurotoxicity. In some aspects, the second therapeutic agent inhibits T cell trafficking, for example, reduces or inhibits immune cells from crossing the blood-brain barrier. Non-limiting examples of such therapeutic agents include antagonists (e.g., antagonistic antibodies) of adhesion molecules on immune cells (e.g., α4 integrin), such as natalizumab. In some aspects, the second therapeutic agent increases the internalization of a sphingosine-1-phosphate (SIP) receptor (e.g., S1PR1 or S1PR5), such as fingolimod or ozanimod. In some aspects, the second therapeutic agent is a nitric oxide synthase (NOS) inhibitor, such as ronopterin, cindunistat, A-84643, ONO-1714, L-NOARG, NCX-456, VAS-2381, GW-273629, NXN-462, CKD-712, KD-7040, or guanidinoethyldisulfide. In some aspects, the second therapeutic agent is an antagonist of CSF1 or CSF1R, such as pexidartinib, emactuzumab, cabiralizumab, LY-3022855, JNJ-40346527, or MCS110. Additional non-limiting examples of the second therapeutic agents include pentosan polysulfate, minocycline, anti-ICAM-1 antibodies, anti-P-selectin antibodies, anti-CD11a antibodies, anti-CD162 antibodies, and anti-IL-6R antibodies (e.g., tocilizumab).

The amount of the fusion protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a fusion protein may be administered during a time when the additional therapeutic agent(s) exerts its prophylactic or therapeutic effect, or vice versa.

### V. Methods of Preparation

In some aspects, the fusion proteins described above can be made using recombinant DNA technology well known to a skilled person in the art. For example, one or more isolated polynucleotides encoding a fusion protein disclosed herein can be ligated to other appropriate nucleotide sequences, including, for example, constant region coding sequences, and expression control sequences, to produce conventional gene expression constructs (i.e., expression vectors) encoding the desired fusion protein. Production of defined gene constructs is within routine skill in the art.

Nucleic acids encoding desired fusion proteins can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the fusion proteins.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The fusion proteins disclosed herein may comprise a single polypeptide chain. In this instance, a host cell can be transfected with a single vector expressing the polypeptide (e.g., containing an expression control sequence operably linked to a nucleotide sequence encoding the polypeptide). Alternatively, the fusion proteins disclosed herein may comprise two or more polypeptides. In this instance, a host cell can be co-transfected with more than one expression vector, for example, one expression vector expressing each polypeptide. A host cell can also be transfected with a single expression vector that expresses the two or more polypeptides. For example, the coding sequences of the two or more polypeptides can be operably linked to different expression control sequences (e.g., promoter, enhancer, and/or internal ribosome entry site (IRES)). The coding sequences of the two or more polypeptides can also be separated by a ribosomal skipping sequence or self-cleaving sequence, such as a 2A peptide.

In some aspects, in order to express a fusion protein, an N-terminal signal sequence is included in the protein construct. Exemplary N-terminal signal sequences include signal sequences from interleukin-2, CD-5, IgG kappa light chain, trypsinogen, serum albumin, and prolactin.

After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of the fusion proteins.

The fusion proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

In some aspects, provided herein is a nucleic acid encoding any of the fusion proteins described herein. In some aspects, provided herein is a vector comprising any of the nucleic acids described herein. In some aspects, provided herein is a recombinant cell comprising any of the nucleic acids described herein. In some aspects, the recombinant cell comprises any of the vectors described herein.

In some aspects, provided herein is a method of producing a fusion protein disclosed herein. In some aspects, the method comprises culturing a cell that comprises any of the nucleic acids described herein under suitable conditions that allow expression of the fusion protein.

In some aspects, the method further comprises isolating the fusion protein.

In some aspects, the method further comprises formulating the fusion protein with a pharmaceutically acceptable carrier or excipient.

### VI. Pharmaceutical Compositions

In some aspects, the present disclosure also features pharmaceutical compositions that contain therapeutically effective amounts of the fusion proteins described herein. In some aspects, the pharmaceutical composition comprises any of the fusion proteins described herein. In some aspects, the pharmaceutical composition comprises any of the nucleic acids described herein. In some aspects, the pharmaceutical composition comprises any of the vectors described herein. In some aspects, the pharmaceutical composition comprises any of the recombinant cells described herein. In some aspects, the pharmaceutical composition comprises a pharmaceutically acceptable carrier or excipient.

The composition can be formulated for use in a variety of drug delivery systems. One or more physiologically acceptable excipients or carriers can also be included in the composition for proper formulation. Suitable formulations for use in the present disclosure are found in Adeboye Adejare, Remington: The Science and Practice of Pharmacy (23d ed. 2020). For a brief review of methods for drug delivery, see, e.g., Langer, Science (1990) 249:1527-1533, 1990.

In some aspects, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In some aspects, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Adeboye Adejare, Remington: The Science and Practice of Pharmacy (23d ed. 2020)).

In some aspects, a pharmaceutical composition may contain nanoparticles, e.g., polymeric nanoparticles, liposomes, or micelles (See Anselmo et al. (2016) BIOENG. TRANSL. MED. 1: 10-29).

In some aspects, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(-)-3-hydroxybutyric acid. Sustained release compositions may also include liposomes that can be prepared by any of several methods known in the art.

Pharmaceutical compositions containing a fusion protein disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In some aspects, a fusion protein disclosed herein is administered by IV infusion. In some aspects, a fusion protein disclosed herein is administered by intratumoral injection. Useful formulations can be prepared by methods known in the pharmaceutical art. For example, see Adeboye Adejare, Remington: The Science and Practice of Pharmacy (23d ed. 2020). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous drug delivery formulation may be contained in a syringe, pen, or bag. In some aspects, the bag may be connected to a channel comprising a tube and/or a needle. In some aspects, the formulation may be a lyophilized formulation or a liquid formulation. In some aspects, the formulation may freeze-dried (lyophilized) and contained in about 12-60 vials. In some aspects, the formulation may be freeze-dried and 45 mg of the freeze-dried formulation may be contained in one vial. In some aspects, the about 40 mg-about 100 mg of freeze-dried formulation may be contained in one vial. In some aspects, freeze dried formulation from 12, 27, or 45 vials are combined to obtain a therapeutic dose of the protein in the intravenous drug formulation. In some aspects, the formulation may be a liquid formulation and stored as about 250 mg/vial to about 1,000 mg/vial. In some aspects, the formulation may be a liquid formulation and stored as about 600 mg/vial. In some aspects, the formulation may be a liquid formulation and stored as about 250 mg/vial.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In some aspects, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In some aspects, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In some aspects, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In some aspects, the buffer system includes citric acid monohydrate, sodium citrate, disodium phosphate dihydrate, and/or sodium dihydrogen phosphate dihydrate. In some aspects, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In some aspects, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. In some aspects, the pH of the formulation is adjusted with sodium hydroxide.

A polyol, which acts as a tonicifier and may stabilize a fusion protein disclosed herein, may also be included in the formulation. The polyol is added to the formulation in an amount which may vary with respect to the desired isotonicity of the formulation. In some aspects, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In some aspects, the polyol which may be used in the formulation as a tonicity agent is mannitol. In some aspects, the mannitol concentration may be about 5 to about 20 mg/ml. In some aspects, the concentration of mannitol may be about 7.5 to 15 mg/ml. In some aspects, the concentration of mannitol may be about 10-14 mg/ml. In some aspects, the concentration of mannitol may be about 12 mg/ml. In some aspects, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In some aspects, the formulation may include a surfactant which is a polysorbate. In some aspects, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In some aspects, the formulation may contain between about 0.1 mg/mL and about 10 mg/mL of polysorbate 80, or between about 0.5 mg/mL and about 5 mg/mL. In some aspects, about 0.1% polysorbate 80 may be added in the formulation.

In some aspects, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In some aspects, the liquid formulation may be diluted with 0.9% saline solution. In some aspects, the liquid formulation of the disclosure may be prepared as a 10 mg/mL concentration solution in combination with a sugar at a stabilizing level. In some aspects the liquid formulation may be prepared in an aqueous carrier. In some aspects, a stabilizer may be added in an amount no greater than that which may result in a viscosity undesirable or unsuitable for intravenous administration. In some aspects, the sugar may be disaccharides, e.g., sucrose. In some aspects, the liquid formulation may also include one or more of a buffering agent, a surfactant, and a preservative.

In some aspects, the pH of the liquid formulation may be set by addition of a pharmaceutically acceptable acid and/or base. In some aspects, the pharmaceutically acceptable acid may be hydrochloric acid. In some aspects, the base may be sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The fusion protein may be lyophilized to produce a lyophilized formulation including the proteins and a lyoprotectant. The lyoprotectant may be sugar, e.g., disaccharides. In some aspects, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized formulation may be in a weight ratio of at least 1:2 protein to sucrose or maltose. In some aspects, the protein to sucrose or maltose weight ratio may be of from 1:2 to 1:5. In some aspects, the pH of the formulation, prior to lyophilization, may be set by addition of a pharmaceutically acceptable acid and/or base. In some aspects the pharmaceutically acceptable acid may be hydrochloric acid. In some aspects, the pharmaceutically acceptable base may be sodium hydroxide. Before lyophilization, the pH of the solution containing the protein of the present disclosure may be adjusted between 6 to 8. In some aspects, the pH range for the lyophilized drug product may be from 7 to 8.

### VI. Kits

The present disclosure also provides kits, or products of manufacture, comprising any of the fusion protein comprising a CD8 antigen binding site and an IL2 protein described herein. One skilled in the art will readily recognize that any of the fusion proteins of the present disclosure can be readily incorporated into one of the established kit formats which are well known in the art.

The following examples are illustrative and do not limit the scope of the claimed aspects.

### EXAMPLES

### Example 1. CD8 Targeted IL2 Constructs

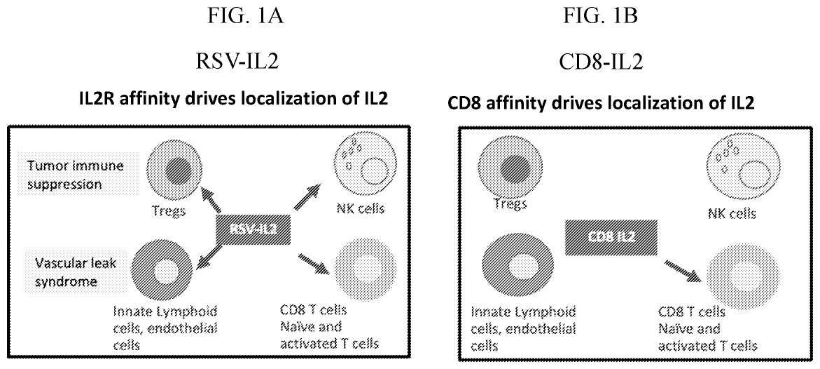

Untargeted RSV-IL2 localization was driven by IL2 affinity, which led to activation of IL2 signaling on all IL2R expressing immune cells (FIG. 1A). In addition to boosting the proliferation and effector function of T and NK cells, it enhanced immune suppressive function of high affinity IL2R(αβγ) expressing Tregs and activated innate lymphoid cells and IL2Rα expressing endothelial cells responsible for causing vascular leak syndrome. Linking IL2 to CD8 antibody resulted in the localization of IL2 based on CD8 antibody affinity instead of IL2 affinity, which helped to load IL2 specifically on CD8 T cells and reduce IL2 mediated toxicity (FIG. 1). The CD8-IL2 targeting improved the therapeutic window by allowing very low Treg expansion and improved expansion of CD8 T cells at low doses.

The CD8-IL2 construct contained different fusion formats. Eight different formats of mouse CD8-IL2 molecules were generated by fusing human IL2 to anti-mouse CD8 antibody or a fragment of anti-mouse CD8 antibody. All anti-mouse CD8 antibodies and antigen-binding fragments used in these molecules were derived from one of the two antagonistic anti-mouse CD8 antibodies YTS105 or YTS169. These eight different molecular formats were evaluated in in vitro to understand how targeting of IL2 to CD8 T cells affects specific binding to CD8 T cell, IL2 loading, IL2 mediated phospho STAT5 activation and proliferation. These CD8-IL2 constructs were also evaluated in vivo to understand how two CD8 binding arm vs. one CD8 binding arm, Fab vs IgG, fusing IL2 to CD8 antibody light chain vs heavy chain affected in vivo efficacy and safety. The molecular formats which induced best in vitro activation and proliferation of CD8 T cells over Tregs and showed better in vivo efficacy and safety profile were used to generate human CD8 targeted IL2 molecules-hCD8 IgG-IL2(LC) and hCD8 IgG-IL2(HC). All anti-human CD8 antigen binding molecules were derived from the anti-human CD8 antibody comprising SEQ ID NOs: 5 (HC) and 6 (LC).

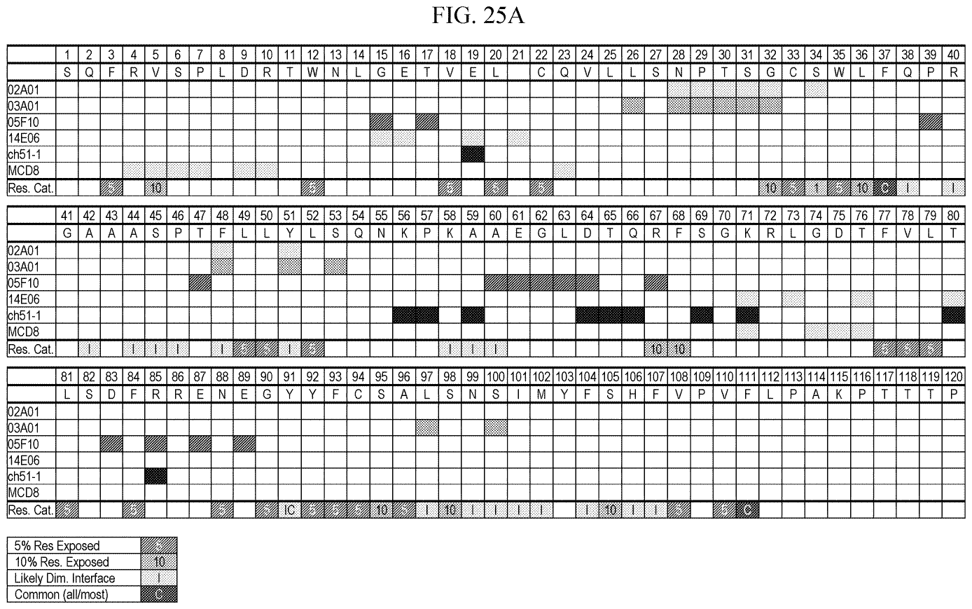

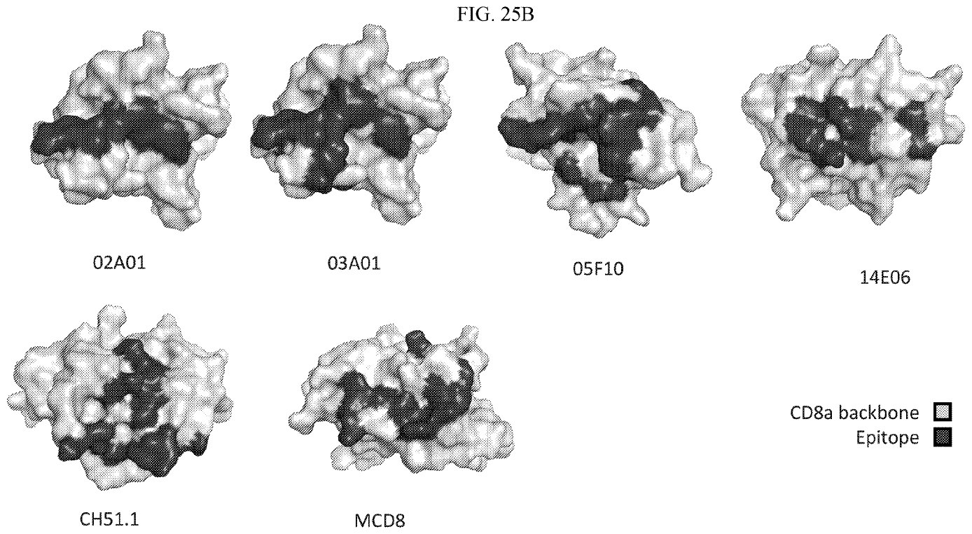

The epitope bound by the anti-CD8 antibody portions was determined by Yeast Surface Display. Heatmaps were generated to show enrichment ratios normalized by wildtype enrichment ratios. Residues that should not be part of an epitope map were denoted and filtered out (e.g., buried residues as determined by 5% and 10% cutoff in solvent-accessible surface area (SASA); paired cysteines; and residues that showed up as hotspots for all antibodies analyzed). The number of substitutions that gave an enrichment ratio greater than 4 was determined, and residues were mapped onto the crystal structure to confirm the resulting map (See FIGS. 25A-25B).

### Example 2. In Vitro Testing of CD8 Targeted IL2 Constructs

### CD8 Binding and IL2 Loading

Anti-mouse CD8-human IL2 molecules were evaluated for their ability to specifically bind and load IL2 on CD8 T cells. Briefly, 1×10⁵ mouse splenocytes were incubated with indicated CD8-IL2 (human FC receptor) molecules from one of the concentrations ranging from 0.05 pM to 500 nM. Anti-mouse CD8 antibody was used as control. Following one hour incubation with molecules, splenocytes were washed twice with RPMI complete medium and once with PBS. The washed splenocytes were stained with anti-TL2 (MQ1-17H12), anti-human kappa (MHK-49) and lineage surface makers CD4(RM4-4), NK1.1(PK136) and CD19(6D5) for 20 minutes, after which the cells were washed and analyzed using flow cytometer. Mean fluorescence intensity data was analyzed using four parameter nonlinear regression method to calculated binding affinity in GraphPad prism.

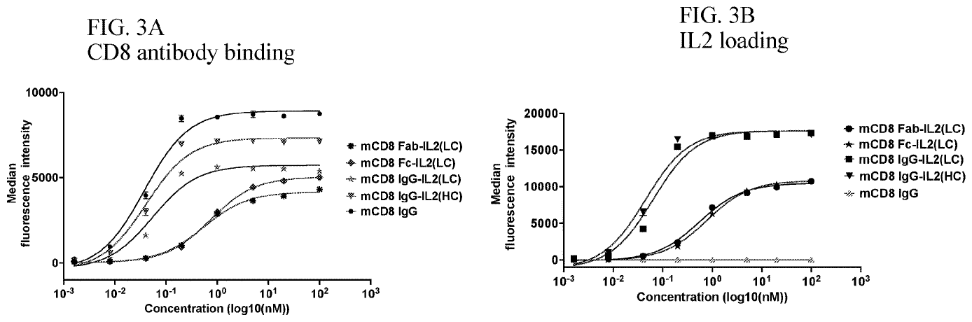

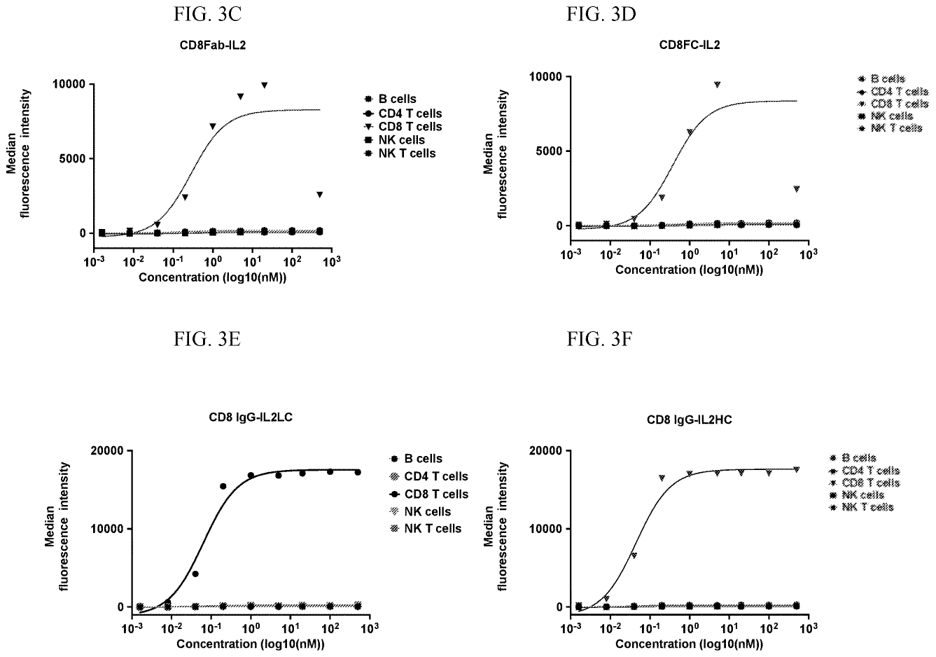

mCD8 IgG-IL2(LC) and mCD8 IgG-IL2(HC) molecules with two CD8 binding domain and two IL2 payloads binds (FIG. 3A, Table 10) and loads IL2 (FIG. 3B, Table 11) on CD8 T cells better than molecules mCD8 Fab-IL2(LC) and mCD8 Fc-IL2(LC) with one CD8 binding domain and one IL2 payload.

TABLE 10
CD8 antibody binding

| Constructs | EC 50 |
|---|---|
| mCD8 Fab-IL2(LC) | 0.5386 |
| mCD8 Fc-IL2(LC) | 0.738 |
| mCD8 IgG-IL2(LC) | 0.05477 |
| mCD8 IgG-IL2(HC) | 0.04052 |
| mCD8IgG | 0.03755 |

TABLE 11
IL2 loading

| Constructs | EC 50 |
|---|---|
| mCD8 Fab-IL2(LC) | 0.550 |
| mCD8 Fc-IL2(LC) | 0.7738 |
| mCD8 IgG-IL2(LC) | 0.06404 |
| mCD8 IgG-IL2(HC) | 0.04483 |

All tested CD8 targeted IL2 molecules loads IL2 preferentially on CD8 T cells over NK, CD4 and B cells (FIGS. 3C-3F). CD8-IL2 molecules tested include CD8Fab-IL2 (FIG. 3C), CD8Fc-IL2 (FIG. 3D), CD8IgG-IL2LC (FIG. 3E), and CD8 IgG-IL2HC (FIG. 3F). pSTAT5 activation

Mouse CD8 targeted IL2 and untargeted IL2 molecules were evaluated for their potency to activate pSTAT5 in CD8 T cells over Tregs and NK cells. Mouse splenocytes (1×10⁶ cells) were thawed and cultured with RPMI media at 37° C. before co-culturing with CD8-IL2 and untargeted IL2 protein for 30 minutes. Subsequently, cells were washed and stained with surface markers CD3(17A2), CD4(RM4-5) and NK1.1(PK136) for 20 minutes. Cells were then washed twice, fixed and permeabilized following manufacture protocol (BD Biosciences, Transcription Factor Phospho Buffer Set-15844409) and stained with internal markers Foxp3(MF-14) and pSTAT5(clone 47/Stat5(pY694). After 45 minutes of incubation with antibodies, cells were washed twice and analyzed on a flow cytometer. The percentage of STAT5 phosphorylation was analyzed using four parameter nonlinear regression method to calculate the potency of pSTAT5 activation in GraphPad prism.

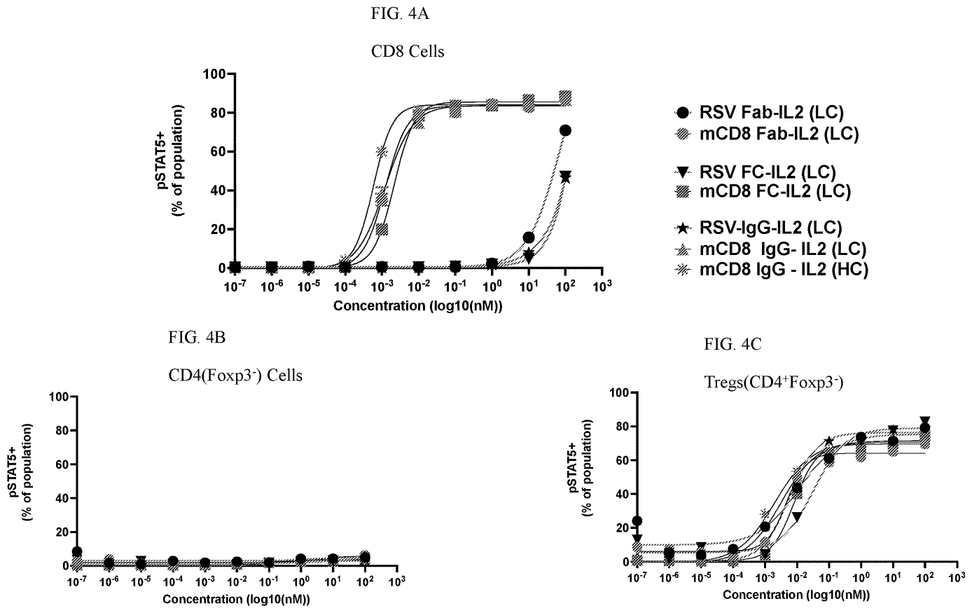

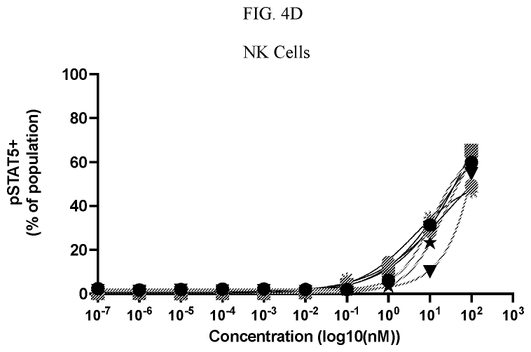

The potency of phospho STAT5 activation in CD8 T cells, Tregs, NK, CD8 and CD4(Foxp3negative) cells in response to treatment of different format CD8-IL2 and untargeted IL2 molecules is shown in Table 12. CD8 targeting of IL2 significantly increase potency of pSTAT5 activation in CD8 T cells and it also decreased pSTAT5 activation in Tregs compared to untargeted IL2 molecules. This led to increased Tregs to CD8 EC50 ratio and preferential activation of CD8 T cells over Tregs when targeting IL2 to CD8 T cells. The percentage of pSTAT5 activation in response to treatment of IL2 molecules is represented in FIGS. 4A-4D for CD8 T cells (FIG. 4A), CD4(Foxp3−) T Cells (FIG. 4B), Tregs(CD4+Foxp3−) (FIG. 4C), and NK cells (FIG. 4D). Ultimately, CD8 targeted IL2 activated pSTAT5 in CD8 T cells about 400,000 times better than untargeted format matched RSV IL2 molecules.

TABLE 12
EC50 of pSTAT5 activation

| | RSV | | | | | | |
|---|---|---|---|---|---|---|---|
| | Fab-IL2 | mCD8 Fab- | RSV FC- | mCD8 FC- | RSV-IgG-IL2 | mCD8 IgG- | mCD8 IgG - |
| | (LC) | IL2 (LC) | IL2 (LC) | IL2 (LC) | (LC) | IL2 (LC) | IL2 (HC) |
| CD8⁺ T cells | 71.48 | 0.001246 | 32555 | 0.002171 | 670334 | 0.001222 | 0.0005887 |
| CD4+ T cells | 1.420 | 3.935 | 10.62 | 0.1846 | 0.1148 | 0.03676 | 0.06205 |
| Tregs | 0.01101 | 0.004686 | 0.03271 | 0.008183 | 0.005806 | 0.003271 | 0.002018 |
| NK cells | 11.46 | 23.07 | 852071 | 137.2 | 22.27 | 23.52 | 4.176 |
| Treg:CD8 | 0.000154 | 3.76 | 0.0000010 | 3.77 | 0.00000000866 | 2.68 | 3.43 |

### CD8 T Cell Expansion

Mouse splenocytes (5×10⁵ cells) were co-cultured with CD8 targeted IL2 and untargeted IL2 molecules in RPMI (+10% FBS) medium for 4 days prior to analyzing the immune cell subsets. Briefly, Cells were washed twice, surface stained with CD4(RM4-4), CD3(17A2) and NK1.1(PK136), then fixed and permeabilized using Foxp3/Transcription Factor staining buffer set (Thermo Fisher Scientific) according to manufacturer's protocol. After permeabilization, cells were stained with Foxp3(FJK-16s), CD8(53-6.7) and Ki67(16A8) for 30 minutes, then washed and analyzed by a flow cytometer.

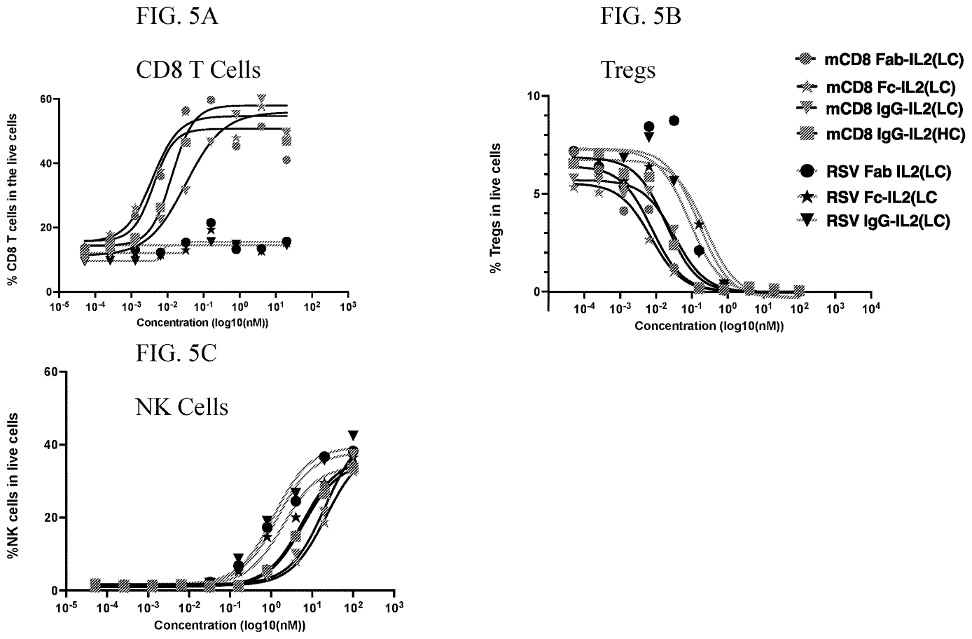

The percent expansion of different immune cell population in live cells were calculated and plotted in response to indicated dose of CD8 targeted IL2 and untargeted IL2 molecules. CD8 targeting of IL2 selectively expands CD8 T cells compared to untargeted IL2 (FIGS. 5A-5C) and Table 13. Also, CD8 targeting reduce the expansion of Tregs, and NK cells.

TABLE 13
Percentage of CD8 T Cell expansion

| Concentration | 32 pm | 160 pm |
|---|---|---|
| mCD8 Fab-IL2(LC) | 56.43 | 59.7 |
| mCD8 Fc-IL2(LC) | 56.13 | 60.63 |
| mCD8 IgG-IL2(LC) | 31.5 | 46.56 |
| mCD8 IgG-IL2(HC) | 46.46 | 63.16 |
| RSV-Fab-IL2(LC) | 15.36 | 21.5 |
| RSV-Fc-IL2(LC) | 13 | 19.33 |
| RSV-IgG-IL2(LC) | 14.73 | 15.56 |

Comparison of mCD8 IgG IL2(HN) and mCD8 IgG IL2(LN) Constructs

The binding affinity of N terminal IL2 fusion mouse CD8 antibody to mouse CD8 T cells were determined as described above. The potency of pSTAT5 activation in CD8 T cells and Tregs in response to co culturing of indicated antibodies with mouse splenocytes were analyzed as described above.

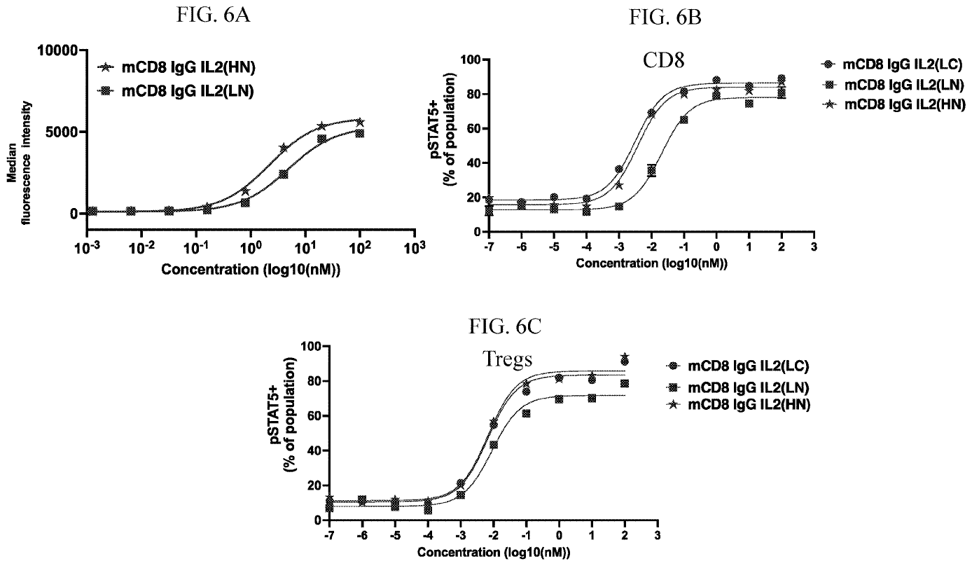

Linking IL2 to N terminus of mouse CD8 antibody heavy chain (mCD8 IgG IL2 (HN)) showed slightly better binding affinity than linking IL2 to N terminus of mouse CD8 antibody light chain (mCD8 IgG IL2 (LN)) (FIG. 6A), Potency of pSTAT5 activation in CD8 T cells (FIG. 6B) and Tregs (FIG. 6C) in response to mCD8 IgG IL2 (HN) molecule treatment in mouse splenocyte is similar to mCD8 IgG IL2 (LC). Among the N terminal IL2 fusion molecules tested mCD8 IgG IL2 (HN) is superior to mCD8 IgG IL2 (LN). Table 14 compares the EC50 of CD8 binding, while Table 15 compares the EC50 of three constructs for pSTAT5 activation.

TABLE 14
mCD8 IgG IL2 (HN) and mCD8 IgG IL2 (LN) CD8 binding

| | mCD8 IgG IL2 (HN) | mCD8 IgG IL2 (LN) |
|---|---|---|
| EC50 | 2.200 | 4.881 |

TABLE 15
mCD8 IgG IL2 (HN), mCD8 IgG IL2 (LN),
and mCD8 IgG IL2 (LC) pSTAT5 activation

| | CD8⁺ | | |
|---|---|---|---|
| EC50 | T cells | Tregs | Treg:CD8 |
| mCD8 IgG IL2 (LC) | 3.12 pM | 6.73 pM | 2.16 |
| mCD8 IgG IL2 (LN) | 20.43 pM | 8.92 pM | 0.44 |
| mCD8 IgG IL2 (HN) | 3.74 pM | 6.75 pM | 1.8 |

### Example 3. In Vivo Testing of CD8 Targeted IL2 Constructs

### CD8 T Cell Expansion

C57BL/6 mice were dosed with HBSS, 2 mg/kg mCD8 IgG IL2 (LC) and RSV IgG IL2 (LC)) through tail vein and 4 days later mice were euthanized, blood and spleen were analyzed by flow cytometry. Mouse CD8 antibody clone YTS169 was used in this experiment. Briefly, Spleen and Blood were lysed with ACK lysis buffer, washed (PBS, 0.5% BSA, 2 mM) EDTA), 1×10⁶ cells along with counting beads (CountBright, Thermofisher) were surface stained with CD3(17A2), CD4(RM4-4), CD8(53-6.7), NK1.1(PK136) then stained for Foxp3 intra cellular marker by following Foxp3/Transcription Factor staining buffer set (Thermo Fisher Scientific) manufacturer's protocol. The cells were analyzed by flow and the number of each population in the spleen was calculated using counting beads.

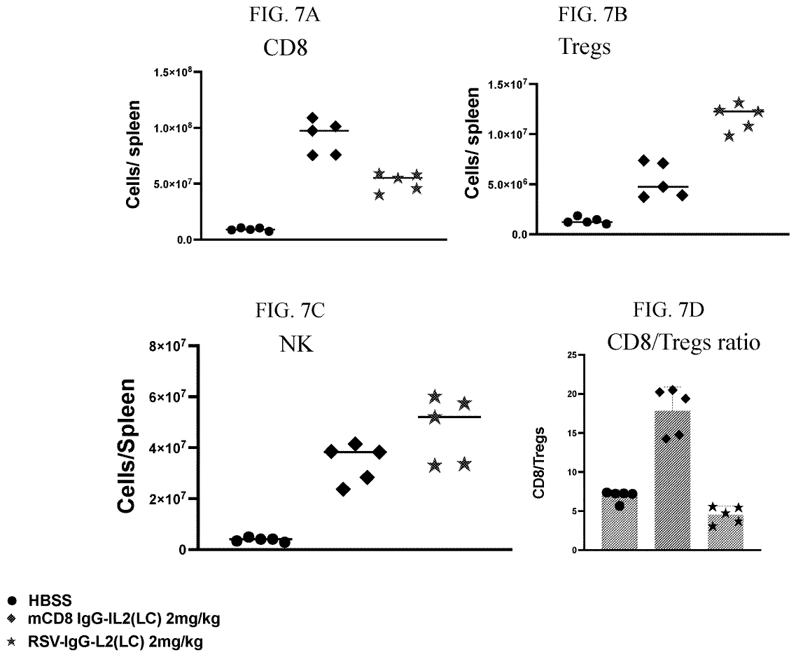

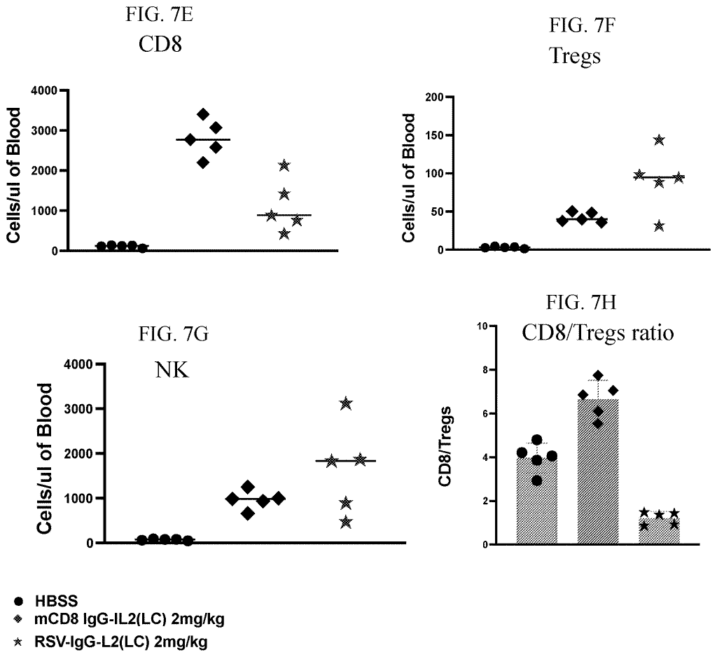

Targeting of IL2 to CD8 T cells expands CD8 T cells over Tregs and NK both in Spleen and blood compared to untargeted IL2, which increase CD8 to Tregs ratio, an important clinical biomarker for better efficacy in cancer patient. Untargeted IL2 significantly expands Tregs compared to CD8 targeted IL2, which reduce CD8 mediated anti-tumor immune response (FIG. 7A and FIG. 7B).

### IL2 Mediated Toxicity in B16F10 Tumor Bearing Mice for Untargeted IL2 and CD8 Targeted IL2

Eight to ten weeks old female C57BL/6 mice were adopted to the facility, shaved and engrafted subcutaneously with 5×10⁵ B16F10 (ATCC, CRL-6475) syngeneic melanoma tumor cells. Mice were randomized on day 8 when tumor reached around 100 mm³ and individual mice were weighed and dosed through tail vein based on the body weight. Forty-eight hours after dosing HBSS, 0.5, 1, 2, or 3 mg/kg of untargeted IL2 (RSV IgG IL2 (LC)) or 1, 2, 3 mg/kg of CD8 targeted IL2 (mCD8 IgG IL2 (LC)), mice serum were collected and analyzed by Luminex using custom made ProcartaPlex multiplex immune assay. Manufacture suggested protocol was followed for staining and analysis. The result was expressed as pg of cytokine per μL of blood. Mouse CD8 antibody clone YTS169 was used in this experiment.

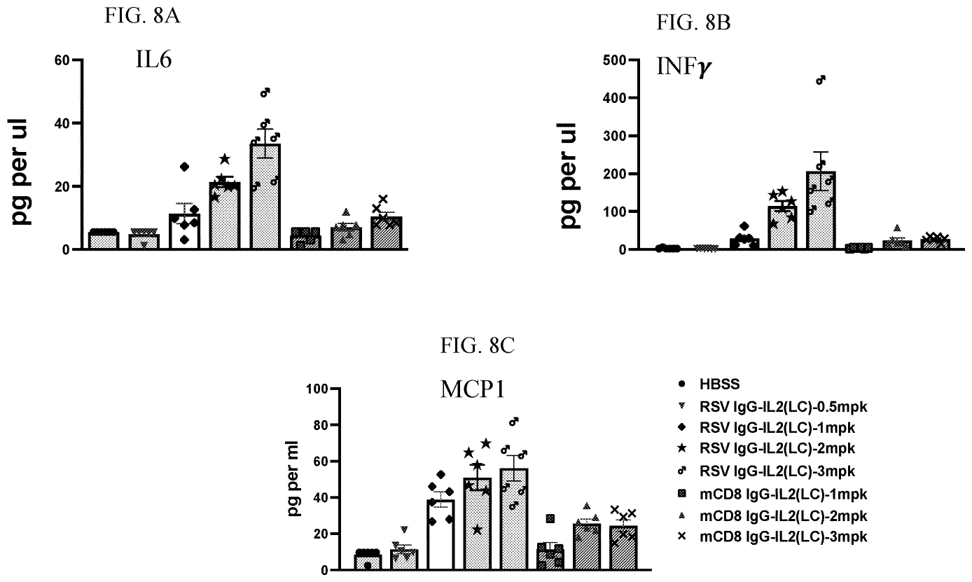

Dosing untargeted RSV-IL2 in B16F10 syngeneic tumor model induced dose dependent increase in inflammatory cytokine such as IL6, INFγ and MCP-1 which are responsible for increased untargeted IL2 mediated toxicity and body weight loss. Targeting wild type IL2 to CD8 T cell significantly reduced IL2 mediated induction of inflammatory cytokine and body weight loss in tumor bearing mice (FIGS. 8A-8C).

Comparison of mCD8 IgG-IL2(LC) and mCD8 Fab-IL2(LC) Constructs

Eight to ten weeks old female C57BL/6 mice were adopted to the facility, shaved and engrafted subcutaneously with 5×10⁵ B16F10 (ATCC, CRL-6475) syngeneic melanoma tumor cells. Mice were randomized on day 8 when tumor reached around 100 mm³. Individual mice were weighed and dosed once in a week through tail vein based on the body weight. Tumor volume and body weight were measured twice a week until tumor reaches maximum volume of 2000 mm³ or until end of the study. When the tumor reached maximum tumor volume or at the end of the study mice were euthanized by CO2 inhalation. mCD8 antibody clone YTS105 (mCD8 Fab IL2 (LC)) and YTS169 (mCD8 IgG IL2 (LC)) were used in this experiment

To analyze expansion of CD8 T cells, 8 to 10 weeks old female C57BL/6 mice were subcutaneously engrafted with 1×10⁶ MC38 syngeneic tumors, on day 8 when tumor reached around 100 mm³, mice were randomized for the study. The blood CD8 T cell number was analyzed 4 days after dosing 2 mg/kg equimolar mCD8 IgG IL2 (LC) and mCD8 Fab IL2 (LC) molecule as described above.

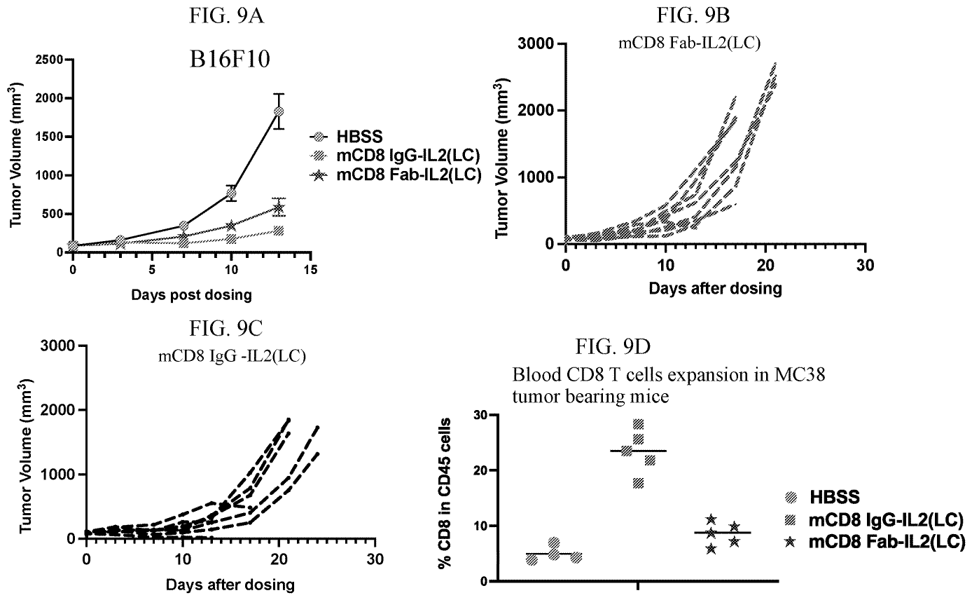

The CD8 IL2 molecule with IgG scaffold shows better efficacy (FIGS. 9A, 9C) and CD8 T cell expansion (FIG. 9D) than CD8-IL2 molecule with Fab scaffold (FIGS. 9A, 9B, and 9D).

### Dose Dependent Tumor Study

The efficacy study in B16F10 tumor was carried out as described above. The B16F10 tumor bearing mice were randomized on day 8 and dosed with 1 mg/kg or 3 mg/kg of mCD8 IgG IL2 (LC) molecules or 3 mg/kg of untargeted IL2 molecules once in a week for 3 times. Tumor volume and body weight were measured twice a week until tumor reaches maximum volume of 2000 mm³.

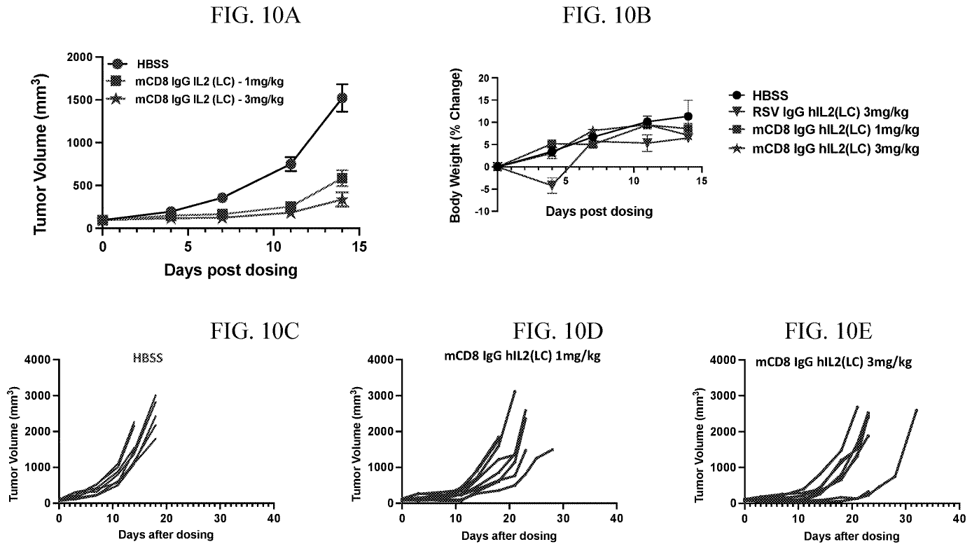

CD8 targeted IL2 molecules induced dose dependent tumor growth inhibition in B16F10 tumor model at 1 mg/kg (FIG. 10D) and 3 mg/kg dose (FIG. 10E), with HBSS alone (FIG. 10C) used as a control (FIG. 10A). Untargeted IL2 at 3 mg/kg dose induced body weight loss, CD8 targeting of IL2 at the same dose reduces toxicity and well tolerated without any body weight loss (FIG. 10B).

### Efficacy and Toxicity Study

The efficacy and toxicity (as measured by body change) of CD8-L2 molecule with two CD8 binding domains (mCD8 IgG IL2 (LC), mCD8 IgG IL2 (HC) and one CD8 binding domain CD8 Fc IL2 (LC) was evaluated in B16F10 tumor bearing mice as indicated above. The tumor bearing mice were dosed 4 times at 6 μg of IL2 equivalent, two times in a week. Tumor volume and body weight were measured twice a week until tumor reaches maximum volume of 2000 mm³.

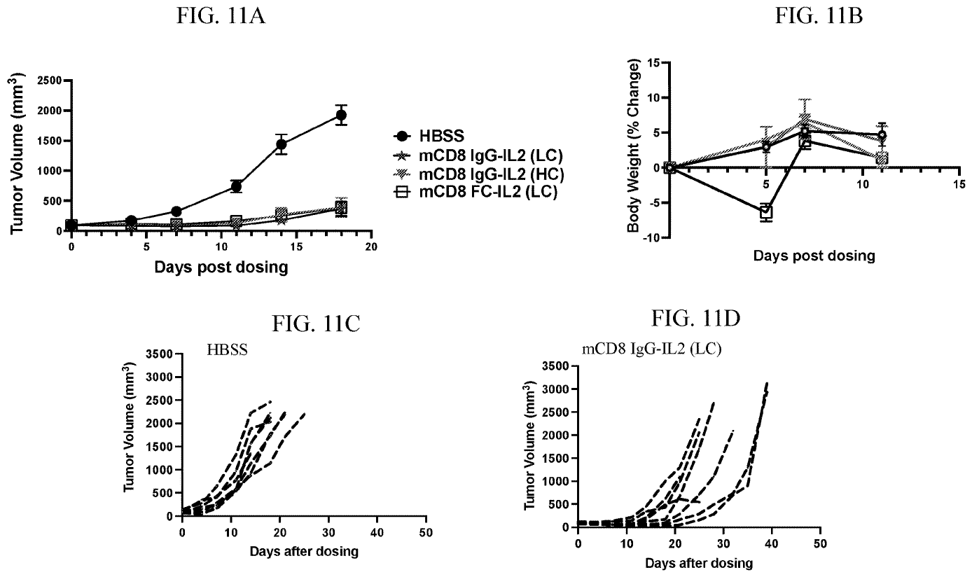

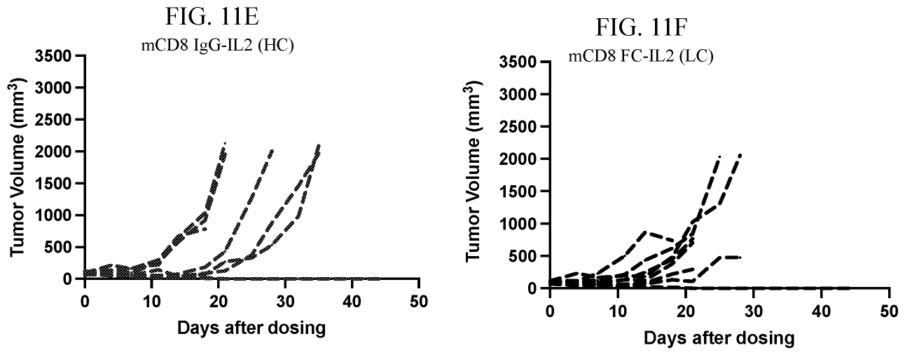

CD8-IL2 molecule with two CD8 binding arm (high affinity) or one CD8 binding arm (low affinity) showed similar efficacy (FIGS. 11A, 11C-11F), but avid binder tolerated well and showed better safety profile (FIG. 111B) compared to molecule with one CD8 binding arm (CD8 Fc IL2 (LC)).

Next, the serum cytokine level in response to untargeted and CD8-IL2 treatment was measured as described above. Briefly, B16F10 tumor bearing mice were dosed with 6.7 μg of IL2 molecules, the blood serum was harvested 48 hours after dosing and analyzed by Luminex and expressed as pg/μL.

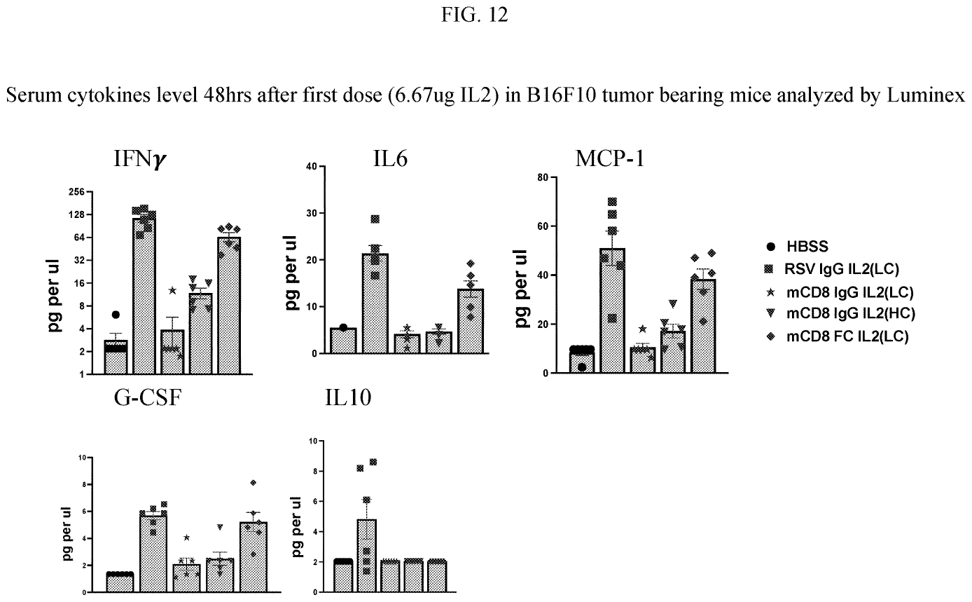

CD8 targeting of IL2 decreases IL2-mediated toxicity due to lower inflammatory cytokine production compared to untargeted RSV-IL2 molecule. CD8 IgG-hIL2 (LC) and CD8 IgG-hIL2 (HC) molecule with avid binding produce lower serum inflammatory cytokines than CD8 Fc-hIL2 (LC) with one CD8 binding domain. Untargeted IL2 molecule activates an immunosuppressive pathway and induces IL-10 production (FIG. 12).

### Example 4. Comparison of mCD8 IgG IL2 (LC) and mCD8 IgG IL2-1×(HC) Constructs

CD8 T cell Binding

To understand how IL2 payload number affects binding affinity of CD8 targeted IL2 molecules, mCD8 IgG IL2 (LC) and mCD8 IgG IL2 1×(HC) molecules at various concentration were co-cultured with mouse splenocytes for 45 minutes and analyzed for IL2 loading by following the method described above.

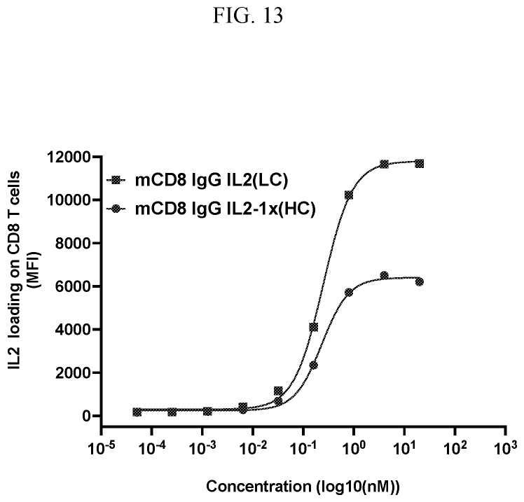

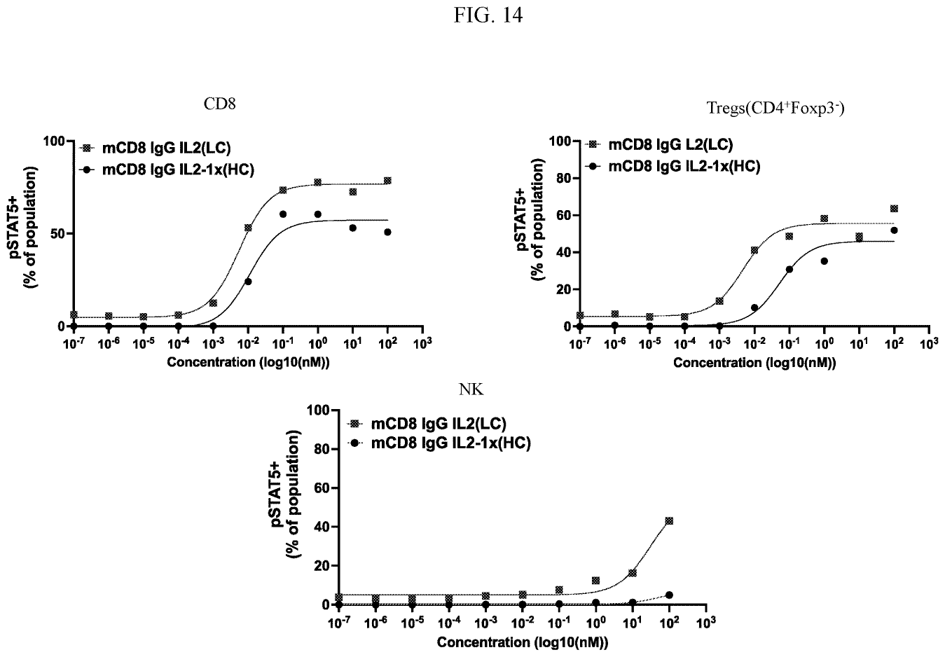

CD8-IL2 molecules with either two IL2 payload (mCD8 IgG IL2 (LC)) or one IL2 payload (mCD8 IgG IL2 1×(HC)) both bound to CD8 T cells at similar affinity. CD8 affinity drove the binding. The molecule with two IL2 payload (mCD8 IgG IL2 (LC) loaded twice the amount of IL2 at saturating concentration compared to molecule with one IL2 payload (FIG. 13) (mCD8 IgG IL2 1×(HC)).

pSTAT5 Activation

Mouse splenocytes were co-cultured with mCD8 IgG IL2 (LC) and mCD8 IgG IL2 1×(HC) and stained for pSTAT5 activation as described above.

The percentage of STAT5 phosphorylation (FIG. 14) and EC50 pSTAT5 activation (Table 16) for mCD8 IgG IL2 (LC) and mCD8 IgG IL2 1×(HC) molecules indicated that the CD8-IL2 molecule with two IL2 payload is better in activating CD8 T cells over one IL2 payload.

TABLE 16
EC50 pSTAT5 activation for mCD8 IgG
IL2 (LC) and CD8 IgG hIL12 1x (HC)

| | mCD8 IgG IL2 (LC) | CD8 IgG hIL2 1x (HC) |
|---|---|---|
| CD8 | 0.005467 | 0.01117 |
| Tregs | 0.004645 | 0.05179 |
| NK | 29.57 | 62.54 |
| Treg:CD8 | 0.85 | 4.64 |

### In Vitro Selective Proliferation of CD8 T Cells Over Tregs and NK Cells

Mouse splenocytes (5×10⁵ cells) were co-cultured with mCD8 IgG IL2 (LC) and mCD8 IgG IL2 1×(HC) molecules in RPMI (+10% FBS) medium for 4 days prior to analyzing the immune cell subsets as described in FIG. 5 method section. The percentage of Ki67 positives cells were represented as percentage of proliferating CD8 T cells, NK and Tregs

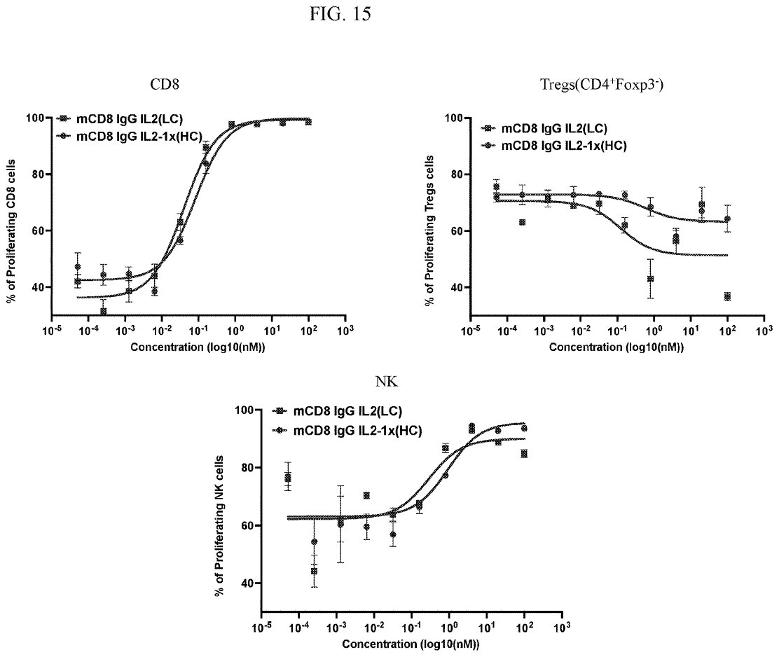

mCD8 IgG IL2 (LC) molecules with two payload increased EC50 of percent proliferation CD8 T cells compared to mCD8 IgG IL2 1×(HC) with one payload. (FIG. 15 and Table 17).

TABLE 17
EC50 of percent proliferation of CD8 T Cells, Tregs, and NK cells

| | mCD8 IgG IL2 (LC) | CD8 IgG IL2-1x (HC) |
|---|---|---|
| CD8 | 0.03965 | 0.08188 |
| Tregs | 0.1225 | 0.5915 |
| NK | 0.2868 | 0.9237 |

### In Vivo Comparison

B16F10 tumor engraftment and measurement were done as described above. Randomized mice were dosed with HBSS, 3 mg/kg of mCD8 IgG IL2 (LC) and mCD8 IgG IL2 1×(HC) molecules once in a week for three time through tail vein based on the body weight.

To analyze efficacy in MC38 tumor model, 8 to 10 weeks old female C57BL/6 mice were subcutaneously engrafted with 1×10⁶ MC38 syngeneic tumors cells, on day 8 when tumor reached around 100 mm³, mice were randomized for the study. Randomized mice were dosed with HBSS, 3 mg/kg of mCD8 IgG IL2 (LC) and mCD8 IgG IL2 1×(HC) molecules once in a week for three time through tail vein based on the body weight. Tumor volume and body weight were measured twice a week until tumor reaches maximum volume of 2000 mm³ or until end of the study. When the tumor reached maximum tumor volume or at the end of the study mice were euthanized by CO2 inhalation.

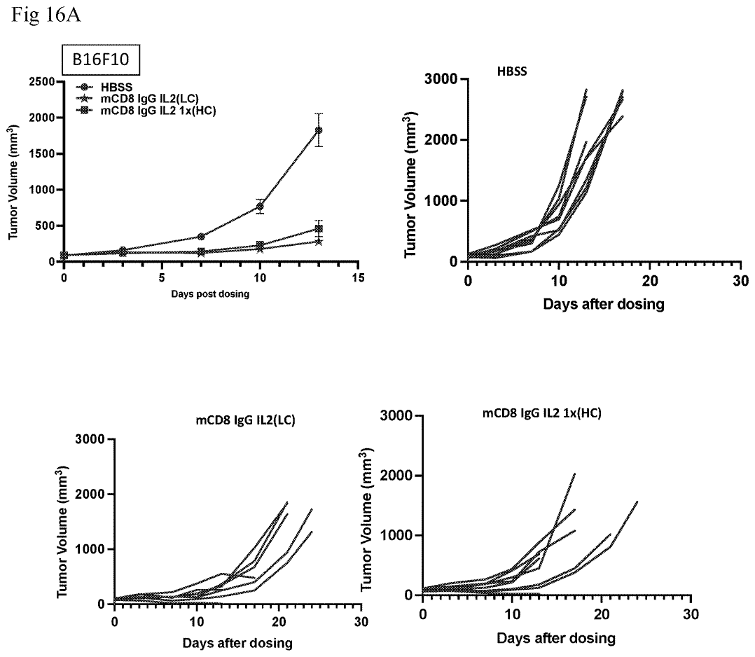

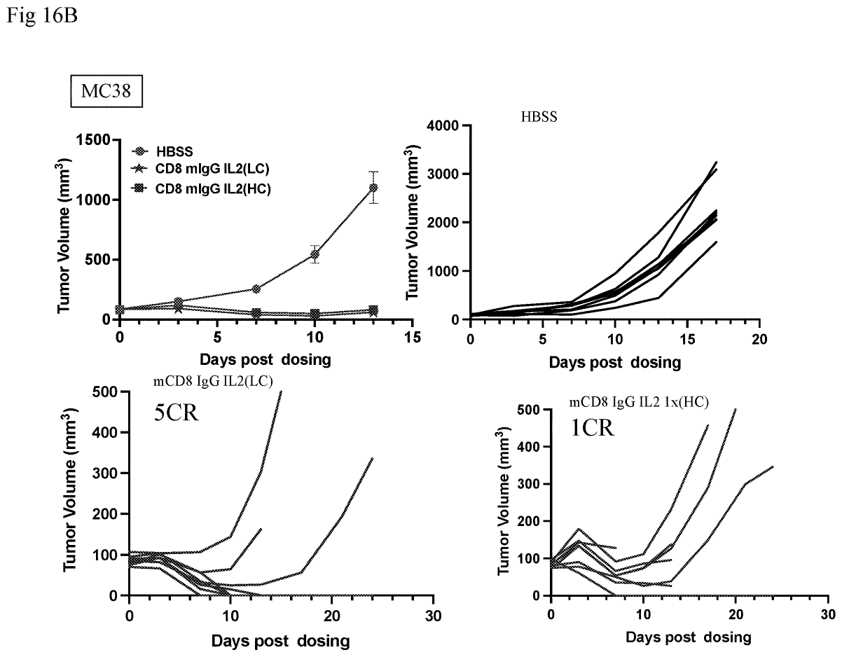

Both mCD8 IgG IL2 (LC) and mCD8 IgG IL2 1×(HC) molecules induced tumor growth inhibition in B16F10 and MC38 tumor models. mCD8 IgG IL2 (LC) treatment gave a better tumor growth inhibition in B16F10 (FIG. 16A) and a more complete response in MC38 (5 complete response (FIG. 16B) than CD8 IgG IL2 1×(HC) (1 complete response) molecules.

### In Vivo Selective Comparison

To analyze expansion of CD8 T cells, 8 to 10 weeks old female C57BL/6 mice were subcutaneously engrafted with 1×10⁶ MC38 syngeneic tumors, on day 8 when tumor reached around 100 mm³, mice were randomized for the study. The blood CD8 T cell, Tregs and NK percentage was analyzed 4 days after dosing 2 mg/kg equimolar mCD8 IgG IL2 (LC) and mCD8 IgG IL2 1×(HC) molecules as described above.

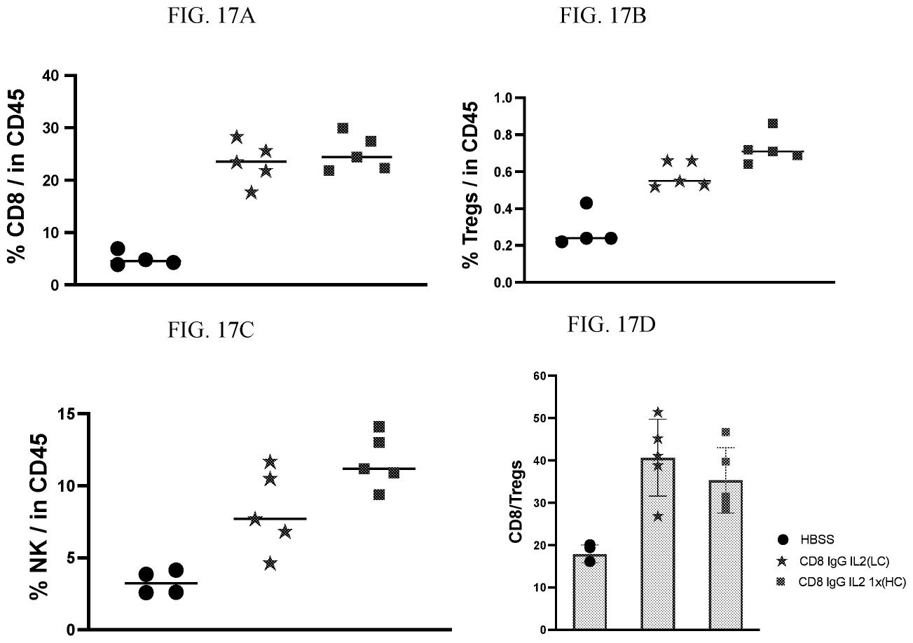

mCD8 IgG IL2 1×(HC) molecule was comparable to mCD8 IgG IL2 (LC) in expanding CD8 T cells. mCD8 IgG IL2 1×(HC) expanded more Tregs and NK cells than mCD8 IgG IL2 (LC), which increased the CD8 to Tregs ratio of mCD8 IgG IL2 (LC) treated mice (FIG. 17).

### Pharmacokinetic Properties

Naïve C57BL/6 mice were dosed with 1 mg/kg of mCD8 IgG IL2 (LC) and mCD8 IgG IL2 1×(HC) molecules through tail vein and blood samples were collected through tail tip micro sampling at 0.5, 1, 6, 24, and 48 hours after dosing for PK analysis. Free mCD8 IgG IL2 (LC) and mCD8 IgG IL2 1×(HC) serum molecules were quantified by sandwich ELISA by capturing the molecules with mouse CD8 protein (50389-M08H, Sino biologics) and detecting with biotin conjugated anti-human IL2 antibody (500-P22BT, PeproTech). The amount of biotin bound molecules were quantified by incubating with HRP conjugated Streptavidin (N-100, Thermo Fisher) and developing with TMB (BioFX® TMB One Component RP, VWR 100359-154). The concentration of serum CD8-IL2 molecules were quantified using 8 point standard curve. The free serum concentration and corresponding time point was fed into PK solver (Non-Compartmental Analysis) and the indicated PK parameters were generated.

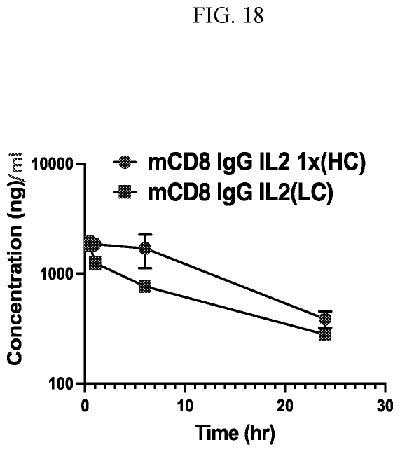

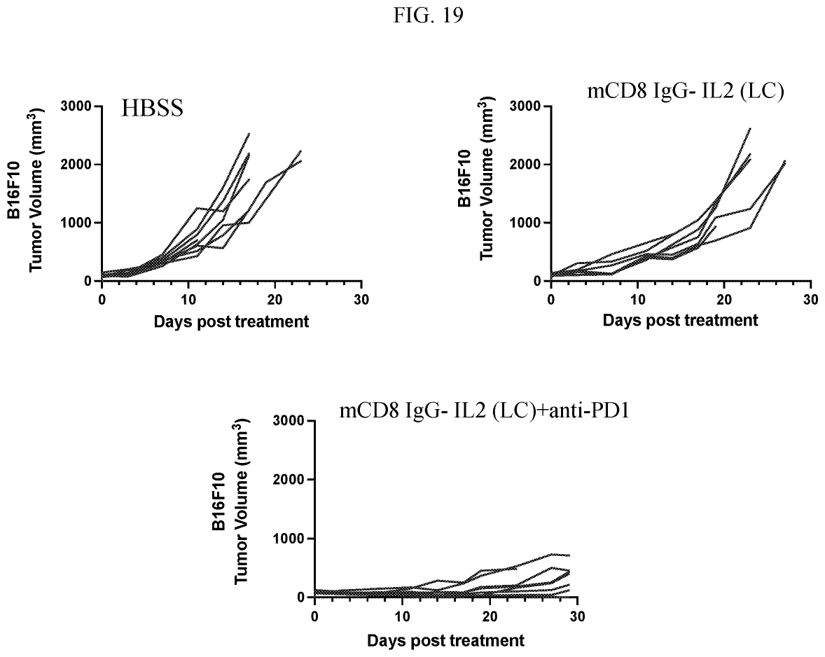

Mean plasma concentration-time profiles and t/2 of mCD8 IgG IL2 (LC) and mCD8 IgG IL2 1×(HC) molecules looked similar (FIG. 18, Table 18). The mCD8 IgG IL2 1×(HC) molecule had lower clearance and higher AUC₀₋ₜ compared to mCD8 IgG IL2 (LC) (Table 9).

TABLE 18
Pharmacokinetic Properties

| | | 1 mg/kg | 1 mg/kg |
|---|---|---|---|
| Parameter | Unit | 2 × 2(LC) | 2 × 1(HC) |
| t_{1/2} | h | 11.0 | 10.0 |
| Cₘₐₓ | ng/ml | 1801.09 | 1968.55 |
| AUC₀₋ₜ | ng/ml*h | 16292.29 | 29544.75 |
| Clearance | (mg/kg)/(ng/ml)/h | 0.000048 | 0.0000285 |

### Example 5. MCD8 IgG IL2 (LC) and Anti-PD1 Combination Therapy

B16F10 tumor engraftment and measurement were done as described above. Randomized mice were dosed with HBSS and 1 mg/kg of mCD8 IgG IL2 (LC) molecules once in a week for two times through tail vein based on the body weight. The anti-PD1 antibody was dosed at 10 mg/kg concentration through intraperitoneum on day 1 and 4 after each mCD8 IgG IL2 (LC) injection.

As shown in FIG. 19, mCD8 IgG IL2 (LC) treatment alone delayed B16F10 tumor growth, and the delay was significantly enhanced with anti-PD1. In the combination group, 7 mice out of 8 mice showed partial response and 1 mice showed a complete response. CD8-IL2 with PD1 combination showed tumor growth delay of 37 (evaluated as time to reach a tumor volume of 1000 mm³) compared to CD8-IL2 (18 days) and HBSS (11 days).

### Example 6. mCD8 IgG IL2 (LC) and Adoptive T Cell Therapy (ACT) Combination

B16F10 tumor engraftment and measurement were done as described above. Tumor bearing mice were randomized and lymphodepleted with cyclophosphamide (CPX, 100 mg/kg) on day −2. On day −1, the mice were engrafted with 2×10⁶ in vitro activated and expanded Pmel-1 T cells. To expand Pmel-1 T cells, splenocytes from Pmel-1 donor mice were lysed with ACK red blood cell lysis buffer, CD8 T cells were isolated with CD8 T cell isolation kit (130-096-495), activated by culturing on CD3 and CD8 coated plate, then expand with IL2 and IL 7 for one day, then maintained in IL 21 containing RPMI media until day 4 before engrafting into B16F10 tumor bearing mice through tail vein. On day 0 when tumor reached around 120 mm³, mice were dosed with HBSS, rIL2 (0.4 mg/kg, once in a day for 3 days every 7 days for 2 cycles, via intraperitoneal injection) or mCD8 IgG IL2 (LC) (1.5 μg IL2/20 μg mice every 7 days, intravenous injection, two times)

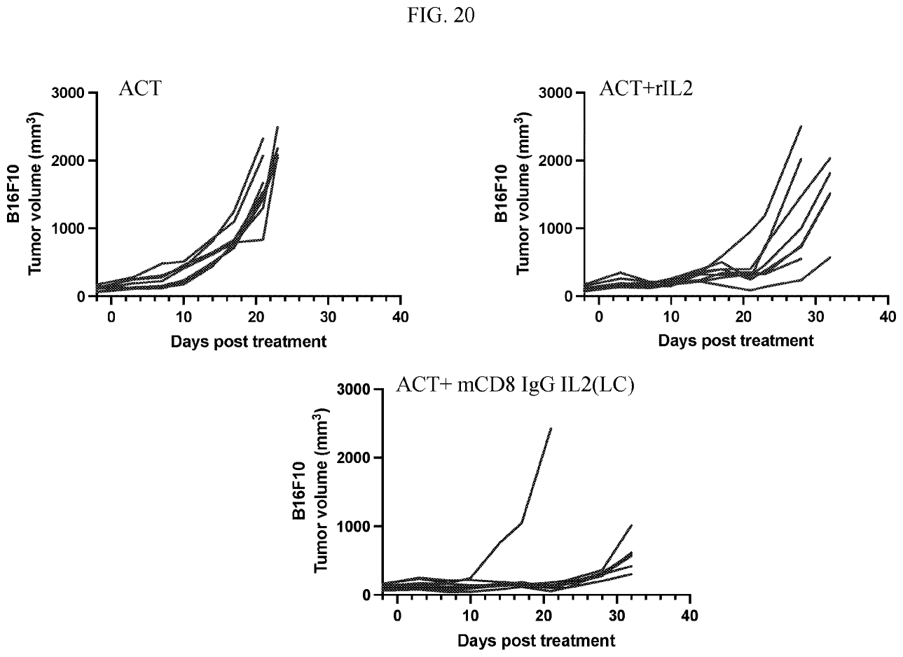

In the aggressive B16F10 tumor models, adoptive cell therapy (ACT) in combination with CD8 targeted IL2 (mCD8 IgG IL2 (LC)) induced significant tumor growth inhibition compared to ACT with rIL2 alone or vehicle. All of the tumor in the ACT+CD8-IL2 combination except for 1 showed median tumor growth delay of 36 days (evaluated as time to reach a tumor volume of 1000 mm³), compared to ACT+IL2 or ACT+vehicle (27 and 18 days respectively) (FIG. 20).

### Example 7. In Vitro Analysis of Human CD8 Targeted IL2 Molecules on Human CD8 T Cells

### CD8 Binding and IL2 Loading

Anti-human CD8-human IL2 molecules were evaluated for their ability to specifically bind and load IL2 on CD8 T cells. Briefly, 1×10′ human PBMC were co-cultured with indicated CD8-IL2 (human FC receptor) molecules from one of the concentrations ranging from 0.05 pM to 0.4 nM. Following one hour incubation with molecules, PBMC were washed twice with RPMI complete medium and washed once with PBS. The washed cells were stained with anti-IL2 (MQ1-17H12), anti-human kappa (MHK-49) and lineage surface markers CD3(UCHT1), CD4(SK3) and NK(HCD56) for 20 minutes, after which the cells were washed and analyzed using a flow cytometer. The binding affinity was quantified and represented as mean fluorescence intensity (MFI).

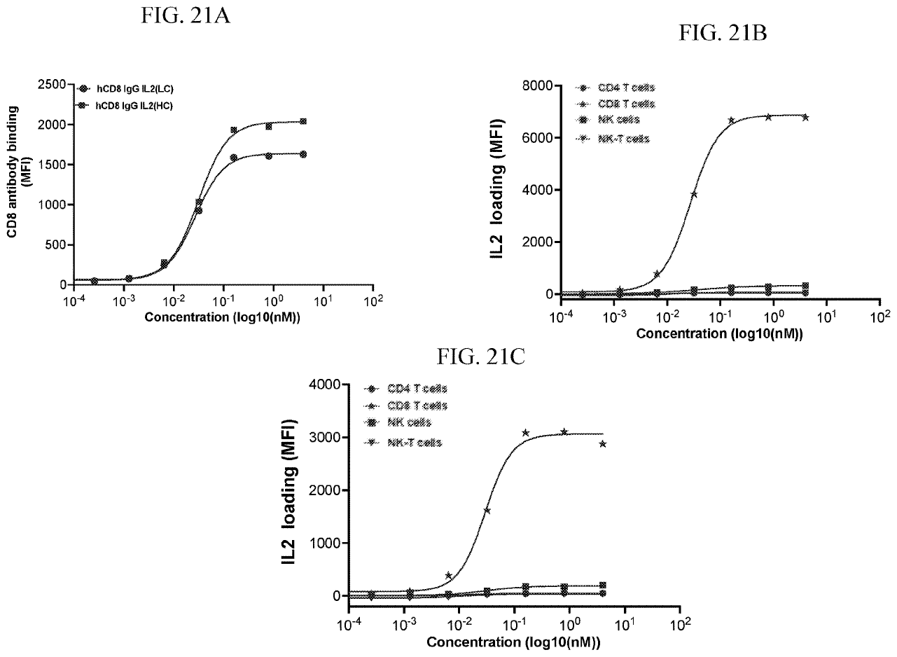

Human CD8-IL2 molecules with either two IL2 payload (hCD8 IgG IL2 (LC)) or one IL2 payload (hCD8 IgG IL2 1×(HC)) both bound to CD8 T cells at similar affinity (FIG. 21A). Human CD8 antibody (VH6/VK3) drove the localization, and both hCD8 IgG IL2 (LC) and hCD8 IgG IL2 1×(HC) loaded IL2 specifically on CD8 T cells over CD4, NK and NK-T cells (FIGS. 21B-21C).

pSTAT5 Activation

Human CD8 targeted IL2 and untargeted IL2 molecules were evaluated for their potency to activate pSTAT5 in CD8 T cells over Tregs and NK cells. Human PBMC (1×10⁶ cells) was cultured with RPMI media at 37° C. for two hours before co-culturing with CD8-IL2 and untargeted IL2 molecules for 30 minutes. Subsequently, cells were washed and stained with surface markers CD8(SK1), CD3(UCHT1), and CD4(SK34) for 20 minutes. Cells were then washed twice, fixed and permeabilized following manufacture protocol (BD Biosciences, Transcription Factor Phospho Buffer Set-15844409) and stained with internal markers CD56(R19-760), Foxp3(259D/C7) and pSTAT5(clone 47/Stat5(pY694). After 45 minutes of incubation with antibodies, cells were washed twice and analyzed on a flow cytometer. The percentage of STAT5 phosphorylation was used to calculate the potency of pSTAT5 activation in GraphPad prism.

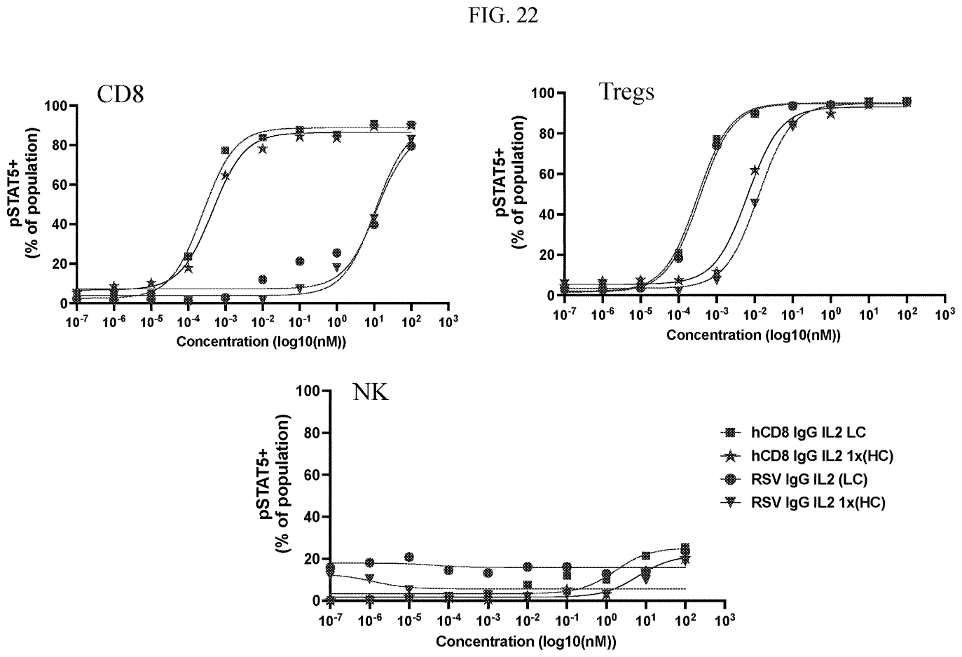

The potency of phospho STAT5 activation in CD8 T cells, Tregs and NK cells in response to treatment of hCD8-IL2 and untargeted IL2 molecules is shown in Table 10. CD8 targeting of IL2 (hCD8 IgG IL2(LC) and hCD8 IgG IL2 1×(HC) significantly increased potency of pSTAT5 activation in CD8 T cells, which led to increased Tregs to CD8 EC50 ratio and preferential activation of CD8 T cells over Tregs (Table 19). The percentage pSTAT5 activation in response to treatment of IL2 molecules is represented in FIG. 22.

TABLE 19
EC50 pSTAT5 Activation for Human CD8 targeted IL2

| | EC50 nM | | | |
|---|---|---|---|---|
| | RSV IgG | hCD8 IgG | RSV IgG | hCD8 IgG |
| | IL2(LC) | IL 2 (LC) | IL2 1x(HC) | IL2 1x(HC) |
| CD8⁺ T cells | 11.52 | 0.0002461 | 10.97 | 0.0004479 |
| Tregs | 0.0003538 | 0.0003115 | 0.01249 | 0.006467 |
| NK cells | 4.153e−005 | 1.679 | 1.212e−006 | 6.243 |
| CD8:Treg | 0.000031 | 1.27 | 0.0011 | 14.44 |

### Selective Proliferation of CD8 T Cells Over Tregs and NK Cells

Human PBMC (4×10⁵) were co-cultured with hCD8 IgG IL2 (LC) and hCD8 IgG IL2 1×(HC)) molecules in X-vivo 10 medium with 5% human serum for 6 days prior to analyzing the immune cell subsets. Briefly, cells were washed twice, surface stained with CD4(SK3), CD3(UCHT1) and CD8(SK1), then fixed and permeabilized using Foxp3/Transcription Factor staining buffer set (Thermo Fisher Scientific) according to manufacturer's protocol. After permeabilization, cells were stained with NK(HCD56), Foxp3(259D) and Ki67(Ki-67) for 30 minutes, then washed and analyzed by a flow cytometer. The percentage Ki67 cells is plotted against concentration, EC50 calculated using four parameter nonlinear regression analysis.

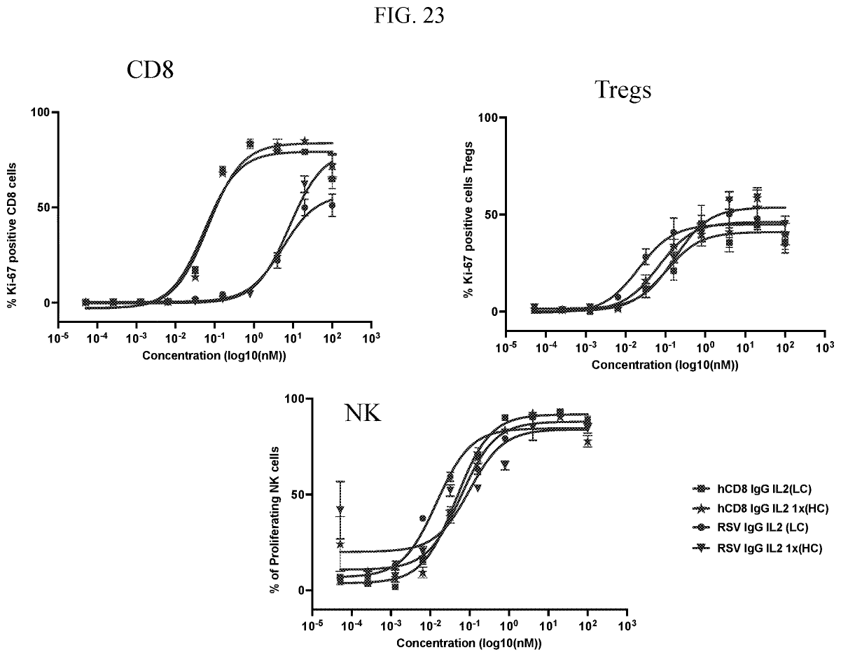

Targeting of IL2 to CD8 T cells increased percentage of Ki67 expression in CD8 T cells compared to untargeted RSV-IL2 molecules (FIG. 23). Both hCD8 IgG IL2 (LC) and hCD8 IgG IL2 1×(HC) molecules significantly reduced EC50 of percentage Ki67 expression in CD8 T cells, while at the same time CD8-IL2 molecules increased EC50 for Tregs and NK Ki67 expression, which led to preferential proliferation of CD8 T cells over Tregs and NK cells in CD8 targeted IL2 treatment compared to untargeted IL2 (Table 20).

TABLE 20
EC50 of Proliferation of CD8 T cells over Tregs and NK cells

| | EC50 nM | | | |
|---|---|---|---|---|
| | RSV IgG | hCD8 IgG | RSV IgG | hCD8 IgG |
| | IL2(LC) | IL2 (LC) | IL2 1x(HC) | IL2 1x(HC) |
| CD8⁺ T cells | 5.395 | 0.05515 | 8.118 | 0.06895 |
| Tregs | 0.02115 | 0.09953 | 0.01249 | 0.02115 |
| NK cells | 0.01435 | 0.04548 | 0.09744 | 0.06063 |
| CD8:Treg | 0.0039203 | 1.805 | 0.002 | 0.307 |

### CD8 T Cell Expansion

Co culturing IL2 molecules with PBMC, staining and data analysis followed as described above. The percentage indicated cells within the live cells were plotted against concentration, and EC50 was calculated using three parameters nonlinear regression analysis

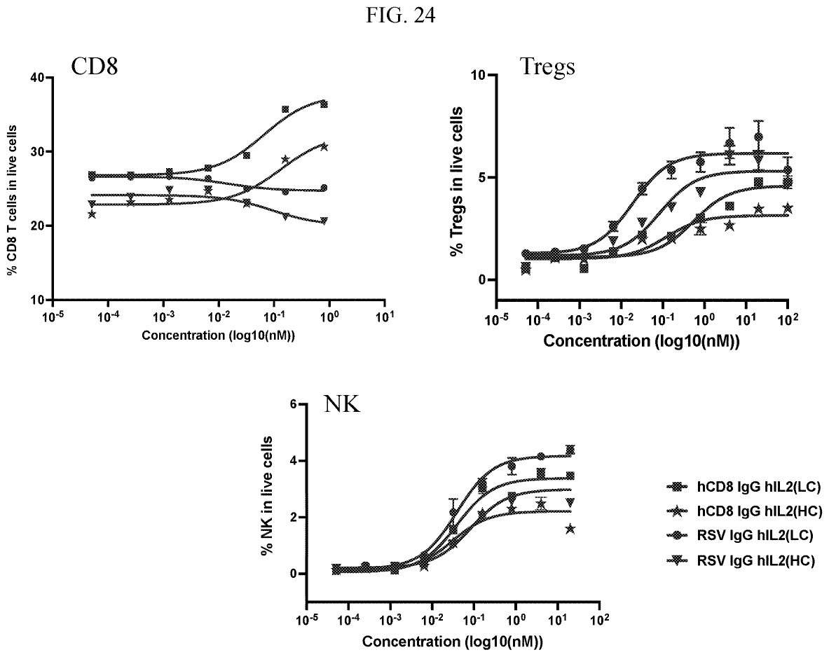

CD8 targeting of IL2 (hCD8 IgG IL2 (LC) and hCD8 IgG IL2 1×(HC)) preferentially expanded CD8 T cells and decreased expansion of Tregs and NK cell compared to untargeted RSV-IL2 molecules (RSV IgG IL2 (LC) and RSV IgG IL2 1×(HC)) (FIG. 24).

### Example 8. In Vivo Analysis of CD8 Targeted IL2 Muteins (Prophetic)

### Tumor Growth Inhibition

The efficacy of hCD8 IgG IL2 (LC), hCD8 IgG IL2 1×(HC) molecules and format matched IL2 muteins molecules in B16F10/MC38 tumor models will be compared.

B16F10 tumor engraftment and measurement will done as described above. Randomized mice will be dosed with HBSS, 2 mg/kg of mCD8 IgG IL2 (LC) and mCD8 IgG IL2 1×(HC) and format matched IL2 muteins molecules once in a week for two time through tail vein based on the body weight.

To analyze efficacy in MC38 tumor model, MC38 tumor engraftment and measurement will done as described above. Randomized mice will be dosed with HBSS, 2 mg/kg of mCD8 IgG IL2 (LC) and mCD8 IgG IL2 1×(HC) and format matched IL2 muteins molecules once in a week for two weeks through tail vein based on the body weight.

The data from this experiment will demonstrate whether IL2 muteins are superior to wild type IL2 in inducing tumor growth inhibition.

### CD8 T Cell Expansion

The effect of hCD8 IgG IL2 (LC), hCD8 IgG IL2 1×(HC), untargeted IL2 molecules and format matched IL2 muteins in expanding CD8 T cells over Tregs and NK in human PBMC engrafted NSG-MHC I/II DKO mice will be analyzed.

NSG-MHC I/II DKO mice will be purchased from the Jackson laboratory. 3 days after engrafting 10×10⁶ human PBMC, mice will be dosed with hCD8 IgG IL2 (LC), hCD8 IgG IL2 1×(HC) molecules and format matched IL2 muteins. 4 days after dosing, mice blood and spleen will be analyzed to understand whether human CD8 targeted IL2 molecules and IL2 mutein specifically expands CD8 T cells over Tregs and NK

The data from this experiment will demonstrate whether human CD8 targeted IL2 is better than untargeted IL2 in specifically expanding CD8 T cells over Tregs and NK. Additionally, this experiment will help to understand the difference between CD8 targeted wild type IL2 and IL2 mutein in expanding CD8 T cells.

### Selective Proliferation

The change in tumor, lymph node, and spleen immune cell population in response hCD8 IgG IL2 (LC), hCD8 IgG IL2 1×(HC) molecules and format matched IL2 muteins molecules in B16F10/MC38 tumor models will be analyzed.

B16F10 tumor engraftment and measurement will done as described above. MC38 tumor engraftment and measurement will done as described above. Randomized mice will be dosed with 2 mg/kg of HBSS, 2 mg/kg of mCD8 IgG IL2 (LC) and mCD8 IgG IL2 1×(HC) and format matched IL2 muteins molecules. Immune cell population will be analyzed after 4 days of treatment in blood, tumor, spleen and lymph node.

The data from this experiment will demonstrate change in immune population in response to CD8-IL2 and CD8-IL2 mutein dosing in the tumor bearing mice.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Sequence Table

| SEQ ID | Description | Sequence | Attributes |
|---|---|---|---|
| 97 | WT IgG1 Fc | DKTHTCPPCPAPELLGGPSVFLFPPKPKDTLMISRTPEVTCVVVDV | |
| | Domain | SHEDPEVKFNWYVDGVEVHNAKTKPREEQYNSTYRVVSVLTVLHQD | |
| | | WLNGKEYKCKVSNKALPAPIEKTISKAKGQPREPQVYTLPPSRDEL | |
| | | TKNQVSLTCLVKGFYPSDIAVEWESNGQPENNYKTTPPVLDSDGSF | |
| | | FLYSKLTVDKSRWQQGNVFSCSVMHEALHNHYTQKSLSLSPG | |
| 99 | (GGGGS)₄ | GGGGSGGGGSGGGGSGGGGS | |
| | linker | | |
| 101 | (GS)₁₀ | GSGSGSGSGSGSGSGSGSGS | |
| 102 | (GGS)₁₀ | GGSGGSGGSGGSGGSGGSGGSGGSGGSGGS | |
| 103 | (GGGS)₁₀ | GGGSGGGSGGGSGGGSGGGSGGGSGGGSGGGSGGGSGGGS | |
| 104 | (GGSG)₁₀ | GGSGGGSGGGSGGGSGGGSGGGSGGGSGGGSGGGSGGGSG | |
| 105 | (GGSGG)₁₀ | GGSGGGGSGGGGSGGGGSGGGGSGGGGSGGGGSGGGGSGGGGSGGG | |
| | | GSGG | |
| 106 | (GGGGS)₁₀ | GGGGSGGGGSGGGGSGGGGSGGGGSGGGGSGGGGSGGGGSGGGGSG | |
| | | GGGS | |
| 107 | (GGGGS)₃ | GGGGSGGGGSGGGGS | |
| 108 | (GGGGS)₂₀ | GGGGSGGGGSGGGGSGGGGSGGGGSGGGGSGGGGSGGGGSGGGGSG | |
| | | GGGSGGGGSGGGGSGGGGSGGGGSGGGGSGGGGSGGGGSGGGGSGG | |
| | | GGSGGGGS | |
| 109 | (GGSGG)₂₀ | GGSGGGGSGGGGSGGGGSGGGGSGGGGSGGGGSGGGGSGGGGSGGG | |
| | | GSGGGGSGGGGSGGGGSGGGGSGGGGSGGGGSGGGGSGGGGSGGGG | |
| | | SGGGGSGG | |
| 110 | Human IL-2 | APTSSSTKKTQLQLEHLLLDLQMILNGINNYKNPKLTRMLTFKFYM | |
| | | PKKATELKHLQCLEEELKPLEEVLNLAQSKNFHLRPRDLISNINVI | |
| | | VLELKGSETTFMCEYADETATIVEFLNRWITFCQSIISTLT | |
| 111 | HC: HC- | EVQLVQSGAEVKKPGATVKISCKASGYTFTDFTMHWVQQAPGKGLE | |
| | 02 (VH6) | WIGLVNPNNGGNIYAEKFQGRVTLTVDTSTSTAYMELSSLRSEDTA | |
| | (02A01 | VYYCGRVPLYTSHGMDYWGQGTLVTVSSASTKGPSVFPLAPSSKST | |
| | VH6, human | SGGTAALGCLVKDYFPEPVTVSWNSGALTSGVHTFPAVLQSSGLYS | |
| | CH1) | LSSVVTVPSSSLGTQTYICNVNHKPSNTKVDKKVEPKSC | |
| 112 | LC: LC-02- | EIVLTQSPATLSLSPGERATISCRASESVESYDIQSMHWYQQKPGQ | |
| | VK3-LM-IL2 | APRLLIYRASHLQSGIPARFSGSGSRTDFTLTISSLEPEDFATYYC | |
| | (D30E, | QQSNEDPLTFGQGTKLEIKRTVAAPSVFIFPPSDEQLKSGTASVVC | |
| | N35Q) | LLNNFYPREAKVQWKVDNALQSGNSQESVTEQDSKDSTYSLSSTLT | |
| | | LSKADYEKHKVYACEVTHQGLSSPVTKSFNRGECGGGGSGGGGSGG | |
| | | GGSGGGGSAPTSSSTKKTQLQLEHLLLDLQMILNGINNYKNPKLTR | |
| | | MLTFKFYMPKKATELKHLQCLEEELKPLEEVLNLAQSKNFHLRPRD | |
| | | LISNINVIVLELKGSETTFMCEYADETATIVEFLNRWITFCQSIIS | |
| | | TLT | |
| 113 | HC1: | EVQLVQSGAEVKKPGATVKISCKASGYTFTDFTMHWVQQAPGKGLE | |
| | HC(k)- | WIGLVNPNNGGNIYAEKFQGRVTLTVDTSTSTAYMELSSLRSEDTA | |
| | 02(VH6)TW. | VYYCGRVPLYTSHGMDYWGQGTLVTVSSASTKGPSVFPLAPSSKST | |
| | LA.LA.NA | SGGTAALGCLVKDYFPEPVTVSWNSGALTSGVHTFPAVLQSSGLYS | |
| | (02A01 | LSSVVTVPSSSLGTQTYICNVNHKPSNTKVDKKVEPKSCDKTHTCP | |
| | VH6, human | PCPAPEAAGGPSVFLFPPKPKDTLMISRTPEVTCVVVDVSHEDPEV | |
| | CH1, CH2- | KFNWYVDGVEVHNAKTKPREEQYASTYRVVSVLTVLHQDWLNGKEY | |
| | CH3) | KCKVSNKALPAPIEKTISKAKGQPREPQVYTLPPSRDELTKNQVSL | |
| | | WCLVKGFYPSDIAVEWESNGQPENNYKTTPPVLDSDGSFFLYSKLT | |
| | | VDKSRWQQGNVFSCSVMHEALHNHYTQKSLSLSPGK | |
| 114 | HC2: | DKTHTCPPCPAPEAAGGPSVFLFPPKPKDTLMISRTPEVTCVVVDV | |
| | HC (h)-CH2- | SHEDPEVKFNWYVDGVEVHNAKTKPREEQYASTYRVVSVLTVLHQD | |
| | CH3- | WLNGKEYKCKVSNKALPAPIEKTISKAKGQPREPQVYTLPPSRDEL | |
| | TS.LA.YV. | TKNQVSLSCAVKGFYPSDIAVEWESNGQPENNYKTTPPVLDSDGSF | |
| | LA.LA.NA | FLVSKLTVDKSRWQQGNVFSCSVMHEALHNHYTQKSLSLSPGK | |
| | (human | | |
| | CH2-CH3) | | |
| 115 | HC: HC- | EVQLVQSGAEVKKPGATVKISCKASGYTFTDFTMHWVQQAPGKGLE | |
| | 02(VH6) | WIGLVNPNNGGNIYAEKFQGRVTLTVDTSTSTAYMELSSLRSEDTA | |
| | LA.LA.NA | VYYCGRVPLYTSHGMDYWGQGTLVTVSSASTKGPSVFPLAPSSKST | |
| | (02A01 | SGGTAALGCLVKDYFPEPVTVSWNSGALTSGVHTFPAVLQSSGLYS | |
| | VH6, human | LSSVVTVPSSSLGTQTYICNVNHKPSNTKVDKKVEPKSCDKTHTCP | |
| | CH1, CH2- | PCPAPEAAGGPSVFLFPPKPKDTLMISRTPEVTCVVVDVSHEDPEV | |
| | CH3) | KFNWYVDGVEVHNAKTKPREEQYASTYRVVSVLTVLHQDWLNGKEY | |
| | | KCKVSNKALPAPIEKTISKAKGQPREPQVYTLPPSRDELTKNQVSL | |
| | | TCLVKGFYPSDIAVEWESNGQPENNYKTTPPVLDSDGSFFLYSKLT | |
| | | VDKSRWQQGNVFSCSVMHEALHNHYTQKSLSLSPGK | |
| 116 | HC: HC- | EVQLVQSGAEVKKPGATVKISCKASGYTFTDFTMHWVQQAPGKGLE | |
| | 02(VH6) | WIGLVNPNNGGNIYAEKFQGRVTLTVDTSTSTAYMELSSLRSEDTA | |
| | LA.LA.NA- | VYYCGRVPLYTSHGMDYWGQGTLVTVSSASTKGPSVFPLAPSSKST | |
| | IL2 | SGGTAALGCLVKDYFPEPVTVSWNSGALTSGVHTFPAVLQSSGLYS | |
| | (02A01 | LSSVVTVPSSSLGTQTYICNVNHKPSNTKVDKKVEPKSCDKTHTCP | |
| | VH6, human | PCPAPEAAGGPSVFLFPPKPKDTLMISRTPEVTCVVVDVSHEDPEV | |
| | CH1, CH2- | KFNWYVDGVEVHNAKTKPREEQYASTYRVVSVLTVLHQDWLNGKEY | |
| | CH3-IL2) | KCKVSNKALPAPIEKTISKAKGQPREPQVYTLPPSRDELTKNQVSL | |
| | | TCLVKGFYPSDIAVEWESNGQPENNYKTTPPVLDSDGSFFLYSKLT | |
| | | VDKSRWQQGNVFSCSVMHEALHNHYTQKSLSLSPGKGGGGSGGGGS | |
| | | GGGGSGGGGSAPTSSSTKKTQLQLEHLLLDLQMILNGINNYKNPKL | |
| | | TRMLTFKFYMPKKATELKHLQCLEEELKPLEEVLNLAQSKNFHLRP | |
| | | RDLISNINVIVLELKGSETTFMCEYADETATIVEFLNRWITFCQSI | |
| | | ISTLT | |
| 117 | LC: LC-02- | EIVLTQSPATLSLSPGERATISCRASESVESYDIQSMHWYQQKPGQ | |
| | VK3-LM | APRLLIYRASHLQSGIPARFSGSGSRTDFTLTISSLEPEDFATYYC | |
| | | QQSNEDPLTFGQGTKLEIKRTVAAPSVFIFPPSDEQLKSGTASVVC | |
| | | LLNNFYPREAKVQWKVDNALQSGNSQESVTEQDSKDSTYSLSSTLT | |
| | | LSKADYEKHKVYACEVTHQGLSSPVTKSFNRGEC | |
| 118 | HC1: | EVQLVQSGAEVKKPGATVKISCKASGYTFTDFTMHWVQQAPGKGLE | |
| | HC(k)- | WIGLVNPNNGGNIYAEKFQGRVTLTVDTSTSTAYMELSSLRSEDTA | |
| | 02(VH6)TW. | VYYCGRVPLYTSHGMDYWGQGTLVTVSSASTKGPSVFPLAPSSKST | |
| | LA.LA.NA- | SGGTAALGCLVKDYFPEPVTVSWNSGALTSGVHTFPAVLQSSGLYS | |
| | IL2 | LSSVVTVPSSSLGTQTYICNVNHKPSNTKVDKKVEPKSCDKTHTCP | |
| | (02A01 | PCPAPEAAGGPSVFLFPPKPKDTLMISRTPEVTCVVVDVSHEDPEV | |
| | VH6, human, | KFNWYVDGVEVHNAKTKPREEQYASTYRVVSVLTVLHQDWLNGKEY | |
| | CH2 | KCKVSNKALPAPIEKTISKAKGQPREPQVYTLPPSRDELTKNQVSL | |
| | CH3-IL2) | WCLVKGFYPSDIAVEWESNGQPENNYKTTPPVLDSDGSFFLYSKLT | |
| | | VDKSRWQQGNVFSCSVMHEALHNHYTQKSLSLSPGKGGGGSGGGGS | |
| | | GGGGSGGGGSAPTSSSTKKTQLQLEHLLLDLQMILNGINNYKNPKL | |
| | | TRMLTFKFYMPKKATELKHLQCLEEELKPLEEVLNLAQSKNFHLRP | |
| | | RDLISNINVIVLELKGSETTFMCEYADETATIVEFLNRWITFCQSI | |
| | | ISTLT | |
| 119 | HC2: | EVQLVQSGAEVKKPGATVKISCKASGYTFTDFTMHWVQQAPGKGLE | |
| | HC(h)- | WIGLVNPNNGGNIYAEKFQGRVTLTVDTSTSTAYMELSSLRSEDTA | |
| | 02(VH6)TS. | VYYCGRVPLYTSHGMDYWGQGTLVTVSSASTKGPSVFPLAPSSKST | |
| | LA.YV.LA. | SGGTAALGCLVKDYFPEPVTVSWNSGALTSGVHTFPAVLQSSGLYS | |
| | LA.NA | LSSVVTVPSSSLGTQTYICNVNHKPSNTKVDKKVEPKSCDKTHTCP | |
| | (02A01 | PCPAPEAAGGPSVFLFPPKPKDTLMISRTPEVTCVVVDVSHEDPEV | |
| | VH6, human | KFNWYVDGVEVHNAKTKPREEQYASTYRVVSVLTVLHQDWLNGKEY | |
| | CH1, CH2- | KCKVSNKALPAPIEKTISKAKGQPREPQVYTLPPSRDELTKNQVSL | |
| | CH3) | SCAVKGFYPSDIAVEWESNGQPENNYKTTPPVLDSDGSFFLVSKLT | |
| | | VDKSRWQQGNVFSCSVMHEALHNHYTQKSLSLSPGK | |
| 120 | HC: IL2- | APTSSSTKKTQLQLEHLLLDLQMILNGINNYKNPKLTRMLTFKFYM | |
| | HC-02(VH6) | PKKATELKHLQCLEEELKPLEEVLNLAQSKNFHLRPRDLISNINVI | |
| | LA.LA.NA | VLELKGSETTFMCEYADETATIVEFLNRWITFCQSIISTLTGGGGS | |
| | (IL2-02A01 | GGGGSGGGGSGGGGSEVQLVQSGAEVKKPGATVKISCKASGYTFTD | |
| | VH6, human | FTMHWVQQAPGKGLEWIGLVNPNNGGNIYAEKFQGRVTLTVDTSTS | |
| | CH1, CH2- | TAYMELSSLRSEDTAVYYCGRVPLYTSHGMDYWGQGTLVTVSSAST | |
| | CH3) | KGPSVFPLAPSSKSTSGGTAALGCLVKDYFPEPVTVSWNSGALTSG | |
| | | VHTFPAVLQSSGLYSLSSVVTVPSSSLGTQTYICNVNHKPSNTKVD | |
| | | KKVEPKSCDKTHTCPPCPAPEAAGGPSVFLFPPKPKDTLMISRTPE | |
| | | VTCVVVDVSHEDPEVKFNWYVDGVEVHNAKTKPREEQYASTYRVVS | |
| | | VLTVLHQDWLNGKEYKCKVSNKALPAPIEKTISKAKGQPREPQVYT | |
| | | LPPSRDELTKNQVSLTCLVKGFYPSDIAVEWESNGQPENNYKTTPP | |
| | | VLDSDGSFFLYSKLTVDKSRWQQGNVFSCSVMHEALHNHYTQKSLS | |
| | | LSPGK | |
| 121 | LC: IL2- | APTSSSTKKTQLQLEHLLLDLQMILNGINNYKNPKLTRMLTFKFYM | |
| | LC-02-VK3- | PKKATELKHLQCLEEELKPLEEVLNLAQSKNFHLRPRDLISNINVI | |
| | LM | VLELKGSETTFMCEYADETATIVEFLNRWITFCQSIISTLTGGGGS | |
| | (D30E, N35Q) | GGGGSGGGGSGGGGSEIVLTQSPATLSLSPGERATISCRASESVES | |
| | | YDIQSMHWYQQKPGQAPRLLIYRASHLQSGIPARFSGSGSRTDFTL | |
| | | TISSLEPEDFATYYCQQSNEDPLTFGQGTKLEIKRTVAAPSVFIFP | |
| | | PSDEQLKSGTASVVCLLNNFYPREAKVQWKVDNALQSGNSQESVTE | |
| | | QDSKDSTYSLSSTLTLSKADYEKHKVYACEVTHQGLSSPVTKSFNR | |
| | | GEC | |
| 122 | HC2: IL2- | APTSSSTKKTQLQLEHLLLDLQMILNGINNYKNPKLTRMLTFKFYM | |
| | HC(h)- | PKKATELKHLQCLEEELKPLEEVLNLAQSKNFHLRPRDLISNINVI | |
| | 02(VH6)TS. | VLELKGSETTFMCEYADETATIVEFLNRWITFCQSIISTLTGGGGS | |
| | LA.YV.LA. | GGGGSGGGGSGGGGSEVQLVQSGAEVKKPGATVKISCKASGYTFTD | |
| | LA.NA | FTMHWVQQAPGKGLEWIGLVNPNNGGNIYAEKFQGRVTLTVDTSTS | |
| | (IL2-02A01 | TAYMELSSLRSEDTAVYYCGRVPLYTSHGMDYWGQGTLVTVSSAST | |
| | VH6, human | KGPSVFPLAPSSKSTSGGTAALGCLVKDYFPEPVTVSWNSGALTSG | |
| | CH1, CH2- | VHTFPAVLQSSGLYSLSSVVTVPSSSLGTQTYICNVNHKPSNTKVD | |
| | CH3) | KKVEPKSCDKTHTCPPCPAPEAAGGPSVFLFPPKPKDTLMISRTPE | |
| | | VTCVVVDVSHEDPEVKFNWYVDGVEVHNAKTKPREEQYASTYRVVS | |
| | | VLTVLHQDWLNGKEYKCKVSNKALPAPIEKTISKAKGQPREPQVYT | |
| | | LPPSRDELTKNQVSLSCAVKGFYPSDIAVEWESNGQPENNYKTTPP | |
| | | VLDSDGSFFLVSKLTVDKSRWQQGNVFSCSVMHEALHNHYTQKSLS | |
| | | LSPGK | |
| 123 | HC: HC- | EVQLVQSGAEVKKPGATVKISCKASGYTFTDFTMHWVQQAPGKGLE | |
| | 02(VH6)- | WIGLVNPNNGGNIYAEKFQGRVTLTVDTSTSTAYMELSSLRSEDTA | |
| | IL2 | VYYCGRVPLYTSHGMDYWGQGTLVTVSSASTKGPSVFPLAPSSKST | |
| | (02A01 | SGGTAALGCLVKDYFPEPVTVSWNSGALTSGVHTFPAVLQSSGLYS | |
| | VH6, human | LSSVVTVPSSSLGTQTYICNVNHKPSNTKVDKKVEPKSCGGGGSGG | |
| | CH1, IL2) | GGSGGGGSGGGGSAPTSSSTKKTQLQLEHLLLDLQMILNGINNYKN | |
| | | PKLTRMLTFKFYMPKKATELKHLQCLEEELKPLEEVLNLAQSKNFH | |
| | | LRPRDLISNINVIVLELKGSETTFMCEYADETATIVEFLNRWITFC | |
| | | QSIISTLT | |
| 124 | HC2: | DKTHTCPPCPAPEAAGGPSVFLFPPKPKDTLMISRTPEVTCVVVDV | |
| | HC(h)-CH2- | SHEDPEVKFNWYVDGVEVHNAKTKPREEQYASTYRVVSVLTVLHQD | |
| | CH3- | WLNGKEYKCKVSNKALPAPIEKTISKAKGQPREPQVYTLPPSRDEL | |
| | TS.LA.YV. | TKNQVSLSCAVKGFYPSDIAVEWESNGQPENNYKTTPPVLDSDGSF | |
| | LA.LA.NA- | FLVSKLTVDKSRWQQGNVFSCSVMHEALHNHYTQKSLSLSPGKGGG | |
| | IL2 | GSGGGGSGGGGSGGGGSAPTSSSTKKTQLQLEHLLLDLQMILNGIN | |
| | (human | NYKNPKLTRMLTFKFYMPKKATELKHLQCLEEELKPLEEVLNLAQS | |
| | CH2-CH3- | KNFHLRPRDLISNINVIVLELKGSETTFMCEYADETATIVEFLNRW | |
| | IL2) | ITFCQSIISTLT | |
| 125 | HC(h)- | DKTHTCPPCPAPEAAGGPSVFLFPPKPKDTLMISRTPEVTCVVVDV | |
| | TS.LA.YV. | SHEDPEVKFNWYVDGVEVHNAKTKPREEQYASTYRVVSVLTVLHQD | |
| | LA.LA.PG.NA | WLNGKEYKCKVSNKALGAPIEKTISKAKGQPREPQVYTLPPSRDEL | |
| | | TKNQVSLSCAVKGFYPSDIAVEWESNGQPENNYKTTPPVLDSDGSF | |
| | | FLVSKLTVDKSRWQQGNVFSCSVMHEALHNHYTQKSLSLSPGK | |
| 126 | RSVFabVH | QVTLKESGPTLVKPTQTLTLTCTFSGFSLSTSGMGVSWIRQPPGKA | |
| | | LEWLAHIYWDDDKRYNPSLKSRLTITKDTSKNQVVLTMTNMDPVDT | |
| | | ATYYCARLYGFTYGFAYWGQGTLVTVSSASTKGPSVFPLAPSSKST | |
| | | SGGTAALGCLVKDYFPEPVTVSWNSGALTSGVHTFPAVLQSSGLYS | |
| | | LSSVVTVPSSSLGTQTYICNVNHKPSNTKVDKKVEPKSC* | |
| 127 | RSV-HC(k)- | QVTLKESGPTLVKPTQTLTLTCTFSGFSLSTSGMGVSWIRQPPGKA | |
| | TW | LEWLAHIYWDDDKRYNPSLKSRLTITKDTSKNQVVLTMTNMDPVDT | |
| | LA.LA.PG. | ATYYCARLYGFTYGFAYWGQGTLVTVSSASTKGPSVFPLAPSSKST | |
| | NA | SGGTAALGCLVKDYFPEPVTVSWNSGALTSGVHTFPAVLQSSGLYS | |
| | | LSSVVTVPSSSLGTQTYICNVNHKPSNTKVDKKVEPKSCDKTHTCP | |
| | | PCPAPEAAGGPSVFLFPPKPKDTLMISRTPEVTCVVVDVSHEDPEV | |
| | | KFNWYVDGVEVHNAKTKPREEQYASTYRVVSVLTVLHQDWLNGKEY | |
| | | KCKVSNKALGAPIEKTISKAKGQPREPQVYTLPPSRDELTKNQVSL | |
| | | WCLVKGFYPSDIAVEWESNGQPENNYKTTPPVLDSDGSFFLYSKLT | |
| | | VDKSRWQQGNVFSCSVMHEALHNHYTQKSLSLSPGK* | |
| 128 | VH-RSV- | QVTLKESGPTLVKPTQTLTLTCTFSGFSLSTSGMGVSWIRQPPGKA | |
| | 2aLALAPGNA | LEWLAHIYWDDDKRYNPSLKSRLTITKDTSKNQVVLTMTNMDPVDT | |
| | | ATYYCARLYGFTYGFAYWGQGTLVTVSSAKTTAPSVYPLAPVCGDT | |
| | | TGSSVTLGCLVKGYFPEPVTLTWNSGSLSSGVHTFPAVLQSDLYTL | |
| | | SSSVTVTSSTWPSQSITCNVAHPASSTKVDKKIEPRGPTIKPCPPC | |
| | | KCPAPNAAGGPSVFIFPPKIKDVLMISLSPIVTCVVVDVSEDDPDV | |
| | | QISWFVNNVEVHTAQTQTHREDYNSTLRVVSALPIQHQDWMSGKEF | |
| | | KCKVNNKDLGAPIERTISKPKGSVRAPQVYVLPPPEEEMTKKQVTL | |
| | | TCMVTDFMPEDIYVEWTNNGKTELNYKNTEPVLDSDGSYFMYSKLR | |
| | | VEKKNWVERNSYSCSVVHEGLHNHHTTKSFSRTPGK* | |
| 129 | RSVFab- | DIVMTQSPDSLAVSLGERATINCRASQSVDYNGISYMHWYQQKPGQ | |
| | IL2LC | PPKLLIYAASNPESGVPDRFSGSGSGTDFTLTISSLQAEDVAVYYC | |
| | | QQIIEDPWTFGQGTKVEIKRTVAAPSVFIFPPSDEQLKSGTASVVC | |
| | | LLNNFYPREAKVQWKVDNALQSGNSQESVTEQDSKDSTYSLSSTLT | |
| | | LSKADYEKHKVYACEVTHQGLSSPVTKSFNRGECGGGGSGGGGSGG | |
| | | GGSAPTSSSTKKTQLQLEHLLLDLQMILNGINNYKNPKLTRMLTFK | |
| | | FYMPKKATELKHLQCLEEELKPLEEVLNLAQSKNFHLRPRDLISNI | |
| | | NVIVLELKGSETTFMCEYADETATIVEFLNRWITFCQSIISTLT* | |
| 130 | RSVFab- | DIVMTQSPDSLAVSLGERATINCRASQSVDYNGISYMHWYQQKPGQ | |
| | IL2LC | PPKLLIYAASNPESGVPDRFSGSGSGTDFTLTISSLQAEDVAVYYC | |
| | | QQIIEDPWTFGQGTKVEIKRTVAAPSVFIFPPSDEQLKSGTASVVC | |
| | | LLNNFYPREAKVQWKVDNALQSGNSQESVTEQDSKDSTYSLSSTLT | |
| | | LSKADYEKHKVYACEVTHQGLSSPVTKSFNRGECGGGGSGGGGSGG | |
| | | GGSAPTSSSTKKTQLQLEHLLLDLQMILNGINNYKNPKLTRMLTFK | |
| | | FYMPKKATELKHLQCLEEELKPLEEVLNLAQSKNFHLRPRDLISNI | |
| | | NVIVLELKGSETTFMCEYADETATIVEFLNRWITFCQSIISTLT* | |
| 131 | LC-RSV-mK- | DIVMTQSPDSLAVSLGERATINCRASQSVDYNGISYMHWYQQKPGQ | |
| | hIL2 | PPKLLIYAASNPESGVPDRFSGSGSGTDFTLTISSLQAEDVAVYYC | |
| | | QQIIEDPWTFGQGTKVEIKRADAAPTVSIFPPSSEQLTSGGASVVC | |
| | | FLNNFYPKDINVKWKIDGSERQNGVLNSWTDQDSKDSTYSMSSTLT | |
| | | LTKDEYERHNSYTCEATHKTSTSPIVKSFNRNECGGGGSGGGGSGG | |
| | | GGSAPTSSSTKKTQLQLEHLLLDLQMILNGINNYKNPKLTRMLTFK | |
| | | FYMPKKATELKHLQCLEEELKPLEEVLNLAQSKNFHLRPRDLISNI | |
| | | NVIVLELKGSETTFMCEYADETATIVEFLNRWITFCQSIISTLT* | |
| 132 | RSVHC - | QVTLKESGPTLVKPTQTLTLTCTFSGFSLSTSGMGVSWIRQPPGKA | |
| | LALAPGNA | LEWLAHIYWDDDKRYNPSLKSRLTITKDTSKNQVVLTMTNMDPVDT | |
| | | ATYYCARLYGFTYGFAYWGQGTLVTVSSASTKGPSVFPLAPSSKST | |
| | | SGGTAALGCLVKDYFPEPVTVSWNSGALTSGVHTFPAVLQSSGLYS | |
| | | LSSVVTVPSSSLGTQTYICNVNHKPSNTKVDKKVEPKSCDKTHTCP | |
| | | PCPAPEAAGGPSVFLFPPKPKDTLMISRTPEVTCVVVDVSHEDPEV | |
| | | KFNWYVDGVEVHNAKTKPREEQYASTYRVVSVLTVLHQDWLNGKEY | |
| | | KCKVSNKALGAPIEKTISKAKGQPREPQVYTLPPSRDELTKNQVSL | |
| | | TCLVKGFYPSDIAVEWESNGQPENNYKTTPPVLDSDGSFFLYSKLT | |
| | | VDKSRWQQGNVFSCSVMHEALHNHYTQKSLSLSPGK* | |
| 133 | RSVFab- | DIVMTQSPDSLAVSLGERATINCRASQSVDYNGISYMHWYQQKPGQ | |
| | IL2LC | PPKLLIYAASNPESGVPDRFSGSGSGTDFTLTISSLQAEDVAVYYC | |
| | | QQIIEDPWTFGQGTKVEIKRTVAAPSVFIFPPSDEQLKSGTASVVC | |
| | | LLNNFYPREAKVQWKVDNALQSGNSQESVTEQDSKDSTYSLSSTLT | |
| | | LSKADYEKHKVYACEVTHQGLSSPVTKSFNRGECGGGGSGGGGSGG | |
| | | GGSAPTSSSTKKTQLQLEHLLLDLQMILNGINNYKNPKLTRMLTFK | |
| | | FYMPKKATELKHLQCLEEELKPLEEVLNLAQSKNFHLRPRDLISNI | |
| | | NVIVLELKGSETTFMCEYADETATIVEFLNRWITFCQSIISTLT* | |
| 134 | HC(k)-RSV- | QVTLKESGPTLVKPTQTLTLTCTFSGFSLSTSGMGVSWIRQPPGKA | |
| | TW-(IL2) | LEWLAHIYWDDDKRYNPSLKSRLTITKDTSKNQVVLTMTNMDPVDT | |
| | | ATYYCARLYGFTYGFAYWGQGTLVTVSSASTKGPSVFPLAPSSKST | |
| | | SGGTAALGCLVKDYFPEPVTVSWNSGALTSGVHTFPAVLQSSGLYS | |
| | | LSSVVTVPSSSLGTQTYICNVNHKPSNTKVDKKVEPKSCDKTHTCP | |
| | | PCPAPEAAGGPSVFLFPPKPKDTLMISRTPEVTCVVVDVSHEDPEV | |
| | | KFNWYVDGVEVHNAKTKPREEQYASTYRVVSVLTVLHQDWLNGKEY | |
| | | KCKVSNKALGAPIEKTISKAKGQPREPQVYTLPPSRDELTKNQVSL | |
| | | WCLVKGFYPSDIAVEWESNGQPENNYKTTPPVLDSDGSFFLYSKLT | |
| | | VDKSRWQQGNVFSCSVMHEALHNHYTQKSLSLSPGKGGGSGGGGSG | |
| | | GGGSGGGGSAPTSSSTKKTQLQLEHLLLDLQMILNGINNYKNPKLT | |
| | | RMLTFKFYMPKKATELKHLQCLEEELKPLEEVLNLAQSKNFHLRPR | |
| | | DLISNINVIVLELKGSETTFMCEYADETATIVEFLNRWITFCQSII | |
| | | STLT* | |
| 135 | HC(h)- | QVTLKESGPTLVKPTQTLTLTCTFSGFSLSTSGMGVSWIRQPPGKA | |
| | RSV- | LEWLAHIYWDDDKRYNPSLKSRLTITKDTSKNQVVLTMTNMDPVDT | |
| | TS.LA.YV. | ATYYCARLYGFTYGFAYWGQGTLVTVSSASTKGPSVFPLAPSSKST | |
| | LA.LA.PG.NA | SGGTAALGCLVKDYFPEPVTVSWNSGALTSGVHTFPAVLQSSGLYS | |
| | | LSSVVTVPSSSLGTQTYICNVNHKPSNTKVDKKVEPKSCDKTHTCP | |
| | | PCPAPEAAGGPSVFLFPPKPKDTLMISRTPEVTCVVVDVSHEDPEV | |
| | | KFNWYVDGVEVHNAKTKPREEQYASTYRVVSVLTVLHQDWINGKEY | |
| | | KCKVSNKALGAPIEKTISKAKGQPREPQVYTLPPSRDELTKNQVSL | |
| | | SCAVKGFYPSDIAVEWESNGQPENNYKTTPPVLDSDGSFFLVSKLT | |
| | | VDKSRWQQGNVFSCSVMHEALHNHYTQKSLSLSPGK* | |
| 136 | RSV-VL- | DIVMTQSPDSLAVSLGERATINCRASQSVDYNGISYMHWYQQKPGQ | |
| | hKappa | PPKLLIYAASNPESGVPDRFSGSGSGTDFTLTISSLQAEDVAVYYC | |
| | | QQIIEDPWTFGQGTKVEIKRTVAAPSVFIFPPSDEQLKSGTASVVC | |
| | | LLNNFYPREAKVQWKVDNALQSGNSQESVTEQDSKDSTYSLSSTLT | |
| | | LSKADYEKHKVYACEVTHQGLSSPVTKSFNRGEC* | |
| 137 | CD8 | SQFRVSPLDRTWNLGETVELKCQVLLSNPTSGCSWLFQPRGAAASP | |
| | protein | TFLLYLSQNKPKAAEGLDTQRFSGKRLGDTFVLTLSDFRRENEGYY | |
| | fragment | FCSALSNSIMYFSHFVPVFLPAKPTTTPAPRPPTPAPTIASQPLSL | |
| | (AA 22-182 | RPEACRPAAGGAVHTRGLDFACD | |
| | of WT CD8 | | |
| | NP_001139345.1) | | |
| 138 | CD | MALPVTALLLPLALLLHAARPSQFRVSPLDRTWNLGETVELKCQVL | |
| | NP_001139345.1 | LSNPTSGCSWLFQPRGAAASPTFLLYLSQNKPKAAEGLDTQRFSGK | |
| | | RLGDTFVLTLSDFRRENEGYYFCSALSNSIMYFSHFVPVFLPAKPT | |
| | | TTPAPRPPTPAPTIASQPLSLRPEACRPAAGGAVHTRGLDFACDIY | |
| | | IWAPLAGTCGVLLLSLVITLYCNHRNRRRVCKCPRPVVKSGDKPSL | |
| | | SARYV | |
| 139 | 02A01 | GYTFTDF | |
| | HCDR1- | | |
| | Chothia | | |
| 140 | 02A01 | NPNNGG | |
| | HCDR2- | | |
| | Chothia | | |
| 141 | Humanized | GYTFTDY | |
| | 02A01-VH5 | | |
| | HCDR1- | | |
| | Chothia | | |
| 142 | 03A01 | GYSITSDF | |
| | HCDR1- | | |
| | Chothia | | |
| 143 | 03A01 | SYSGD | |
| | HCDR2- | | |
| | Chothia | | |
| 144 | 03A01 | SYSGE | |
| | HCDR2- | | |
| | 03A01 | | |
| | VH-D64E- | | |
| | Chothia | | |
| 145 | 03A01 | SYSGX1 | wherein |
| | HCDR2 | | X1 is D |
| | consensus- | | or E; |
| | Chothia | | |
| 146 | 05F10 | GFTFSSF | |
| | HCDR1- | | |
| | Chothia | | |
| 147 | 05F10 | SSGDLS | |
| | HCDR2- | | |
| | Chothia | | |
| 148 | 11F05 | GYSITSDY | |
| | HCDR1- | | |
| | Chothia | | |
| 149 | 11F05 | SYSGS | |
| | HCDR2- | | |
| | Chothia | | |
| 150 | 04D08 | GYSFTGY | |
| | HCDR1- | | |
| | Chothia | | |
| 151 | 04D08 | DPYYGG | |
| | HCDR2- | | |
| | Chothia | | |

1. (canceled)

2. A fusion protein comprising i) an antigen-binding portion that binds CD8; and ii) a portion having an IL-2 activity (“IL-2 portion”), wherein the antigen-binding portion comprises an antibody comprising two heavy chains and two light chains and the IL-2 portion comprises two IL-2 proteins, and wherein
(a) each of the IL-2 proteins is fused to one of the C-termini of the two light chains;
(b) each of the IL-2 proteins is fused to one of the N-termini of the two heavy chains;
(c) each of the IL-2 proteins is fused to one of the C-termini of two heavy chains; or
(d) each of the IL-2 proteins is fused to one of the N-termini of the light chains.

3-22. (canceled)

23. The fusion protein of claim 2, wherein each of the heavy chains or heavy chain variable regions in the antigen-binding portion comprises complementarity determining regions HCDR1, HCDR2, and HCDR3 and wherein each of the light chains or light chain variable regions in the antigen-binding portion comprises complementarity determining regions LCDR1, LCDR2, and LCDR3, the HCDR1, HCDR2, HCDR3, LCDR1, LCDR2, and LCDR3 comprise:
(i) the amino acid sequences of SEQ ID NOs: 139, 140, 26, 48, 28, and 29, respectively;
(ii) the amino acid sequences of SEQ ID NOs: 24, 25, 26, 27, 28, or 29, respectively;
(iii) the amino acid sequences of SEQ ID NOs: 139, 140, 26, 27, 28, and 29, respectively; or
(iv) the amino acid sequences of 139, 140, 26, 27, 50, and 29, respectively.

24. (canceled)

25. The fusion protein of claim 2, wherein the antigen-binding portion comprises a heavy chain variable domain (VH) and a light chain variable domain (VL), wherein the VH and the VL comprise;
(i) amino acid sequences at least 95% identical to SEQ ID NOs: 22 and 47; 39 and 47; 40 and 47; 41 and 47; or 42 and 47, respectively;
(ii) amino acid sequences at least 95% identical to SEQ ID NOs; 39 and 45; 40 and 45; 41 and 45; 42 and 45; 22 and 45; 39 and 46; 40 and 46; 41 and 46; 42 and 46; 22 and 46; 39 and 23; 40 and 23; 41 and 23; 42 and 23; or 22 and 23, respectively;
(iii) amino acid sequences at least 95% identical to SEQ ID NOs; 30 and 31, respectively, or 33 and 31, respectively; or
(iv) amino acid sequences at least 95% identical to SEQ ID NOs; 39 and 49; 40 and 49; 41 and 49; 42 and 49; or 22 and 49, respectively.

26-30. (canceled)

31. The fusion protein of claim 2, wherein at least one of the IL-2 protein comprises an amino acid sequence at least 90% identical to SEQ ID NO: 110 and/or wherein the antigen binding portion that binds to CD8 comprises SEQ ID NOs: 5 (HC) and 6 (LC).

32-47. (canceled)

48. The fusion protein of claim 2, wherein the IL-2 portion is linked to the antigen-binding portion by a linker.

49-50. (canceled)

51. A The fusion protein of claim 2, comprising two polypeptide chains, wherein:
(i) the first polypeptide chain comprises the amino acid sequence as set forth in SEQ ID NO: 8 and the second polypeptide chain comprises the amino acid sequence as set forth in SEQ ID NO: 10;
(ii) the first polypeptide chain comprises the amino acid sequence as set forth in SEQ ID NO: 3 and the second polypeptide chain comprises the amino acid sequence as set forth in SEQ ID NO: 17;
(iii) the first polypeptide chain comprises the amino acid sequence as set forth in SEQ ID NO: 5 and the second polypeptide chain comprises the amino acid sequence as set forth in SEQ ID NO: 20;
(iv) the first polypeptide chain comprises the amino acid sequence as set forth in SEQ ID NO: 9 and the second polypeptide chain comprises the amino acid sequence as set forth in SEQ ID NO: 2;
(v) the first polypeptide chain comprises the amino acid sequence as set forth in SEQ ID NO: 13 and the second polypeptide chain comprises the amino acid sequence as set forth in SEQ ID NO: 4;
(vi) the first polypeptide chain comprises the amino acid sequence as set forth in SEQ ID NO: 12 and the second polypeptide chain comprises the amino acid sequence as set forth in SEQ ID NO: 18;
(v) the first polypeptide chain comprises the amino acid sequence as set forth in SEQ ID NO: 112 and the second polypeptide chain comprises the amino acid sequence as set forth in SEQ ID NO: 115;
(vi) the first polypeptide chain comprises the amino acid sequence as set forth in SEQ ID NO: 116 and the second polypeptide chain comprises the amino acid sequence as set forth in SEQ ID NO: 117;
(vii) the first polypeptide chain comprises the amino acid sequence as set forth in SEQ ID NO: 120 and the second polypeptide chain comprises the amino acid sequence as set forth in SEQ ID NO: 117: or
(viii) the first polypeptide chain comprises the amino acid sequence as set forth in SEQ ID NO: 121 and the second polypeptide chain comprises the amino acid sequence as set forth in SEQ ID NO: 115.

52-72. (canceled)

73. The fusion protein of claim 2, wherein the fusion protein has one or more of the following properties: a) activates CD8+ T cells with 10-fold or greater potency as compared to activation of NK cells; b) activates pSTAT5 in CD8+ T cells at similar or greater potency than Tregs; or c) preferentially induces proliferation of CD8+ T cells over Tregs and NK cells.

74. A nucleic acid encoding the fusion protein of claim 2.

75. A vector comprising the nucleic acid of claim 74.

76. A recombinant cell comprising the vector of claim 75.

77. A pharmaceutical composition comprising the fusion protein of claim 2, and a pharmaceutically acceptable carrier or excipient.

78. A method of producing a fusion protein, the method comprising culturing a cell that comprises the nucleic acid of claim 74 under suitable conditions that allow expression of the fusion protein.

79-80. (canceled)

81. A method of preventing or treating a disease or condition in a subject in need thereof, the method comprising administering to the subject the fusion protein of claim 2, wherein the disease or condition is a cancer or an infectious disease.

82. (canceled)

83. The method of claim 8182, wherein the cancer is a bladder cancer, breast cancer, uterine cancer, endometrial carcinoma, ovarian cancer, colorectal cancer, colon cancer, head and neck cancer, lung cancer, stomach cancer, germ cell cancer, bone cancer, squamous cell cancer, skin cancer, neoplasm of the central nervous system, lymphoma, leukemia, sarcoma, virus-related cancer, small-cell lung cancer, non-small cell lung cancer, gastrointestinal cancer, Hodgkin's or non-Hodgkin's lymphoma, pancreatic cancer, glioblastoma, glioma, cervical cancer, liver cancer, myeloma, salivary gland carcinoma, kidney cancer, basal cell carcinoma, melanoma, prostate cancer, vulval cancer, thyroid cancer, testicular cancer, esophageal cancer, and any combinations thereof.

84-85. (canceled)

86. The method of claim 81, further comprising administering an anti-cancer agent.

87. The method of claim 86, wherein the anti-cancer agent comprises an immunomodulator, an adoptive cell therapy, a checkpoint inhibitor, a small molecule based anti-cancer therapy, an antibody based cancer therapy, a cancer vaccine, or a chemotherapeutic agent.

88. The method of claim 86, wherein the immunomodulator is an immunostimulator.

89. The method of claim 88, wherein the immunostimulator comprises an immunostimulatory cytokine, an agonist of a costimulatory molecule, and/or an inhibitor of an immune checkpoint protein, wherein the immunostimulatory cytokine is selected from the group consisting of IL-12, IL-15, IL-2, IL-6, IL-7, IL-18, IL-21, IL-23, and IL-27, and any combinations thereof.

90. (canceled)

91. The method of claim 87, wherein the immunomodulator comprises an immunosuppressor, wherein the immunosuppressor comprises an immunosuppressive cytokine, an inhibitor of a costimulatory molecule, and/or an agonist of an immune checkpoint protein.

92. (canceled)

93. The method of claim 87, wherein the checkpoint inhibitor is selected from the group consisting of a PD-1 antagonist, a PD-L1 antagonist, a LAG-3 antagonist, a TIM-3 antagonist, a CTLA-4 antagonist, a B7-H3 antagonist, a B7-H4 antagonist, a BTLA antagonist, a KIR antagonist, a VISTA antagonist, a TIGIT antagonist, an IDO antagonist, a Siglec-15 antagonist, a GAL-9 antagonist, an HVEM antagonist, or combinations thereof.

94-105. (canceled)


SEQUENCE LISTING (151 sequences)

SEQ ID NO: 1 - protein, 220 aa, synthetic construct
  source 1..220: note = YTS105 Heavy Chain
EVQLKESGPGLVQPSQTLSLTCTVSGFSLTSNSVHWVRQPPGKGLEWMGGIWGDGDTDYNSALKSRLSISRDTSKNQVFLKMNSLQTDDTAIYFCTPLIGSWYFDFWGPGTMVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 214 aa, synthetic construct
  source 1..214: note = YTS105 Light Chain
DIVMTQSPSSLAVSAGERVTLNCKASQNVRNNIAWYQQKPGQSPKLLIYYASYRYTGVPDRFTGDGFGTDFTLAINSVQADDAAFYYCQRIYNSPYTFGAGTKLELIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 454 aa, synthetic construct
  source 1..454: note = YTS169 Heavy Chain
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 213 aa, synthetic construct
  source 1..213: note = YTS169 Light Chain
DIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 450 aa, synthetic construct
  source 1..450: note = Humanized 02A01-VH6-Full IgG Heavy Chain
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 218 aa, synthetic construct
  source 1..218: note = Humanized 02A01-VK3-Full IgG Light Chain
EIVLTQSPATLSLSPGERATISCRASESVDSYDINSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 447 aa, synthetic construct
  source 1..447: note = YTS105HC(k)-TW LA.LA.PG.NA
EVQLKESGPGLVQPSQTLSLTCTVSGFSLTSNSVHWVRQPPGKGLEWMGGIWGDGDTDYNSALKSRLSISRDTSKNQVFLKMNSLQTDDTAIYFCTPLIGSWYFDFWGPGTMVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 447 aa, synthetic construct
  source 1..447: note = YTS105(LALAPGNA)HC
EVQLKESGPGLVQPSQTLSLTCTVSGFSLTSNSVHWVRQPPGKGLEWMGGIWGDGDTDYNSALKSRLSISRDTSKNQVFLKMNSLQTDDTAIYFCTPLIGSWYFDFWGPGTMVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 595 aa, synthetic construct
  source 1..595: note = HC-YTS105-LALAPGNA-hIL2
EVQLKESGPGLVQPSQTLSLTCTVSGFSLTSNSVHWVRQPPGKGLEWMGGIWGDGDTDYNSALKSRLSISRDTSKNQVFLKMNSLQTDDTAIYFCTPLIGSWYFDFWGPGTMVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 362 aa, synthetic construct
  source 1..362: note = YTS105-IL2 LC
DIVMTQSPSSLAVSAGERVTLNCKASQNVRNNIAWYQQKPGQSPKLLIYYASYRYTGVPDRFTGDGFGTDFTLAINSVQADDAAFYYCQRIYNSPYTFGAGTKLELIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 11 - protein, 602 aa, synthetic construct
  source 1..602: note = YTS169- H4A mIgG2a-IL-2
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYASTLRVVSALPIQHQDWMSGKEFKCKVNNKDLGAPIERTISKPKGSVRAPQVYVLPPPEKEMTKKQVSLTCLVKDFMPEDIYVEWTNNGKTELNYKNTEPVLKSDGSYFMYSKLTVEKKNWVERNSYSCSVVHEGLHNHHTTKSFSRTPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 454 aa, synthetic construct; identical to SEQ ID NO: 3
  source 1..454: note = YTS169 VH- mIgG2a LALA

SEQ ID NO: 13 - protein, 602 aa, synthetic construct
  source 1..602: note = hIL2- (GGGGS)3 YTS169 VH- mIgG2a LALA
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 602 aa, synthetic construct
  source 1..602: note = hIL2- (GGGGS)3 YTS169 VH-k-mIgG2a LALAPGNA
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYASTLRVVSALPIQHQDWMSGKEFKCKVNNKDLGAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - protein, 454 aa, synthetic construct
  source 1..454: note = YTS169-H4B-mIgG2a
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYASTLRVVSALPIQHQDWMSGKEFKCKVNNKDLGAPIERTISKPKGSVRAPQVYVLPPPEEEMTKKQVSLTCLVKDFMPEDIYVEWTNNGKTELNYKNTEPVLDSDGSYFMYSELTVEKKNWVERNSYSCSVVHEGLHNHHTTDSFSRTPGK

SEQ ID NO: 16 - protein, 454 aa, synthetic construct
  source 1..454: note = YTS169 VH-h-mIgG2a LALAPGNA
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYASTLRVVSALPIQHQDWMSGKEFKCKVNNKDLGAPIERTISKPKGSVRAPQVYVLPPPEEEMTKKQVTLACAVTDFMPEDIYVEWTNNGKTELNYKNTEPVLDSDGSYFMVSKLRVEKKNWVERNSYSCSVVHEGLHNHHTTKSFSRTPGK

SEQ ID NO: 17 - protein, 361 aa, synthetic construct
  source 1..361: note = LC-YTS169-mK-hIL2
DIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 361 aa, synthetic construct
  source 1..361: note = hIL2- (GGGGS)3- YTS169-VL- mKappa
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 602 aa, synthetic construct
  source 1..602: note = HC(k)-02(VH6)TW-(IL2)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - protein, 371 aa, synthetic construct
  source 1..371: note = LC-02-VK3-IL2
EIVLTQSPATLSLSPGERATISCRASESVDSYDINSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 21 - protein, 450 aa, synthetic construct
  source 1..450: note = HC(h)- 02(VH6)-TS.LA.YV.LA.LA.PG.NA
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 120 aa, synthetic construct
  source 1..120: note = Humanized 02A01-VH6
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSS

SEQ ID NO: 23 - protein, 111 aa, synthetic construct
  source 1..111: note = Humanized 02A01-VK3
EIVLTQSPATLSLSPGERATISCRASESVDSYDINSMHWYQQKPGQAPRLLIYRASHLQSGIPARFSGSGSRTDFTLTISSLEPEDFATYYCQQSNEDPLTFGQGTKLEIK

SEQ ID NO: 24 - protein, 5 aa, synthetic construct
  source 1..5: note = HCDR1 02A01-VH6
DFTMH

SEQ ID NO: 25 - protein, 17 aa, synthetic construct
  source 1..17: note = HCDR2 02A01-VH6
LVNPNNGGNIYAEKFQG

SEQ ID NO: 26 - protein, 11 aa, synthetic construct
  source 1..11: note = HCDR3 02A01-VH6 - Chothia
VPLYTSHGMDY

SEQ ID NO: 27 - protein, 15 aa, synthetic construct
  source 1..15: note = LCDR1 02A01-VK3
RASESVDSYDINSMH

SEQ ID NO: 28 - protein, 7 aa, synthetic construct
  source 1..7: note = LCDR2 02A01-VK3
RASHLQS

SEQ ID NO: 29 - protein, 9 aa, synthetic construct
  source 1..9: note = LCDR3 02A01-VK3 - Chothia
QQSNEDPLT

SEQ ID NO: 30 - protein, 120 aa, synthetic construct
  source 1..120: note = Murine 02A01 VH
EVQLQQSGPELVKPGASLKISCKASGYTFTDFTMHWVKQSHGKSLEWIGLVNPNNGGNIYNQNFKGKATLTVDTSSSTAYMELRSLTSEDSSVYYCGRVPLYTSHGMDYWGQGTSVTVSS

SEQ ID NO: 31 - protein, 111 aa, synthetic construct
  source 1..111: note = Murine 02A01 VL
DIVLTQSPAFLAVSLGQRATISCRASESVDSYDINSMHWYQQKPGQPPKLLIYRASHLQSGIPARFSGSGSRTDFTLTINFVEADDVATYYCQQSNEDPLTFGAGTKLELK

SEQ ID NO: 32 - protein, 17 aa, synthetic construct
  source 1..17: note = Murine 02A01 HCDR2
LVNPNNGGNIYNQNFKG

SEQ ID NO: 33 - protein, 120 aa, synthetic construct
  source 1..120: note = Murine 02A01 VH-M39L
EVQLQQSGPELVKPGASLKISCKASGYTFTDFTLHWVKQSHGKSLEWIGLVNPNNGGNIYNQNFKGKATLTVDTSSSTAYMELRSLTSEDSSVYYCGRVPLYTSHGMDYWGQGTSVTVSS

SEQ ID NO: 34 - protein, 5 aa, synthetic construct
  source 1..5: note = Murine 02A01 VH-M39L HCDR1
DFTLH

SEQ ID NO: 35 - protein, 5 aa, synthetic construct
  source 1..5: note = 02A01 Consensus 1 HCDR1
  VARIANT 4: note = wherein X is M or L
DFTXH

SEQ ID NO: 36 - protein, 17 aa, synthetic construct
  source 1..17: note = 02A01 Consensus 1 HCDR2
  VARIANT 12: note = wherein X is N or A
  VARIANT 16: note = wherein X is K or Q
  VARIANT 14: note = wherein X is N or K
LVNPNNGGNIYXZXFXG

SEQ ID NO: 37 - protein, 15 aa, synthetic construct
  source 1..15: note = 02A01 Consensus 1 LCDR1
  VARIANT 7: note = wherein X is D or E
  VARIANT 12: note = wherein X is N or Q
RASESVXSYDIXSMH

SEQ ID NO: 38 - protein, 17 aa, synthetic construct
  source 1..17: note = 02A01 Consensus 2 HCDR2
  VARIANT 1..17: note = LVNPNNGGNIYAEKFQG
  VARIANT 1..17: note = LVNPNNGGNIYNQNFKG
LVNPNNGGNIYXXXFXG

SEQ ID NO: 39 - protein, 120 aa, synthetic construct
  source 1..120: note = Humanized 02A01-VH1
EVQLVQSGPEVVKPGATLKISCKASGYTFTDFTMHWVKQAPGKSLEWIGLVNPNNGGNIYNQNFKGRATLTVDTSTSTAYMELSSLRSEDTSVYYCGRVPLYTSHGMDYWGQGTLVTVSS

SEQ ID NO: 40 - protein, 120 aa, synthetic construct
  source 1..120: note = Humanized 02A01-VH2
EVQLVQSGAEVKKPGATLKISCKASGYTFTDFTMHWVQQAPGKGLEWIGLVNPNNGGNIYNQNFKGRATLTVDTSTSTAYMELSSLRSEDTSVYYCGRVPLYTSHGMDYWGQGTLVTVSS

SEQ ID NO: 41 - protein, 120 aa, synthetic construct
  source 1..120: note = Humanized 02A01-VH3
EVQLVQSGAEVKKPGATLKISCKASGYTFTDFTMHWVQQAPGKGLEWIGLVNPNNGGNIYAEKFQGRATLTVDTSTSTAYMELSSLRSEDTSVYYCGRVPLYTSHGMDYWGQGTLVTVSS

SEQ ID NO: 42 - protein, 120 aa, synthetic construct
  source 1..120: note = Humanized 02A01-VH4
EVQLVQSGAEVKKPGATVKISCKASGYTFTDFTMHWVQQAPGKGLEWIGLVNPNNGGNIYAEKFQGRATLTVDTSTSTAYMELSSLRSEDTAVYYCGRVPLYTSHGMDYWGQGTLVTVSS

SEQ ID NO: 43 - protein, 120 aa, synthetic construct
  source 1..120: note = Humanized 02A01-VH5
EVQLVQSGAEVKKPGATLKISCKASGYTFTDYYMHWVQQAPGKGLEWIGLVNPNNGGNIYNQNFKGRATLTVDTSTSTAYMELSSLRSEDTSVYYCGRVPLYTSHGMDYWGQGTLVTVSS

SEQ ID NO: 44 - protein, 5 aa, synthetic construct
  source 1..5: note = Humanized 02A01-VH5 HCDR1
DYYMH

SEQ ID NO: 45 - protein, 111 aa, synthetic construct
  source 1..111: note = Humanized 02A01-VK1
EIVLTQSPATLSVSPGERATISCRASESVDSYDINSMHWYQQKPGQPPRLLIYRASHLQSGIPARFSGSGSRTDFTLTISSVEPEDFATYYCQQSNEDPLTFGQGTKLEIK

SEQ ID NO: 46 - protein, 111 aa, synthetic construct
  source 1..111: note = Humanized 02A01-VK2
EIVLTQSPATLSVSPGERATISCRASESVDSYDINSMHWYQQKPGQAPRLLIYRASHLQSGIPARFSGSGSRTDFTLTISSVEPEDFATYYCQQSNEDPLTFGQGTKLEIK

SEQ ID NO: 47 - protein, 111 aa, synthetic construct
  source 1..111: note = Humanized 02A01-VK3 (D30E, N35Q)
EIVLTQSPATLSLSPGERATISCRASESVESYDIQSMHWYQQKPGQAPRLLIYRASHLQSGIPARFSGSGSRTDFTLTISSLEPEDFATYYCQQSNEDPLTFGQGTKLEIK

SEQ ID NO: 48 - protein, 15 aa, synthetic construct
  source 1..15: note = Humanized 02A01-VK3 (D30E, N35Q) LCDR1
RASESVESYDIQSMH

SEQ ID NO: 49 - protein, 111 aa, synthetic construct
  source 1..111: note = Humanized 02A01-VK4
EIVLTQSPATLSVSPGERATISCRASESVDSYDINSMHWYQQKPGQAPRLLIYDASNRATGIPARFSGSGSRTDFTLTISSVEPEDFATYYCQQSNEDPLTFGQGTKLEIK

SEQ ID NO: 50 - protein, 7 aa, synthetic construct
  source 1..7: note = Humanized 02A01-VK4 LCDR2
DASNRAT

SEQ ID NO: 51 - protein, 120 aa, synthetic construct
  source 1..120: note = 03A01 VH
DVQFQESGPGLVKPSQSLSLTCTVTGYSITSDFAWNWIRQFPGNELEWMGYISYSGDTNYNPSLRSRISITRDTSKNQFFLQLDSVTTEDTATYYCTIRGYYTDSAFVFWGQGTLVTVSA

SEQ ID NO: 52 - protein, 107 aa, synthetic construct
  source 1..107: note = 03A01 VL
DIQMTQSSSYLSVSLGGRVTITCKASDHINNWLAWYQQKPGNAPRLLISGATSLETGVPSRFTGSGSGLDYTLSITSLQTEDVATYYCQQYWNSPFTFGSGTKLEIK

SEQ ID NO: 53 - protein, 6 aa, synthetic construct
  source 1..6: note = 03A01 HCDR1
SDFAWN

SEQ ID NO: 54 - protein, 16 aa, synthetic construct
  source 1..16: note = 03A01 HCDR2
YISYSGDTNYNPSLRS

SEQ ID NO: 55 - protein, 11 aa, synthetic construct
  source 1..11: note = 03A01 HCDR3
RGYYTDSAFVF

SEQ ID NO: 56 - protein, 11 aa, synthetic construct
  source 1..11: note = 03A01 LCDR1
KASDHINNWLA

SEQ ID NO: 57 - protein, 7 aa, synthetic construct
  source 1..7: note = 03A01 LCDR2
GATSLET

SEQ ID NO: 58 - protein, 9 aa, synthetic construct
  source 1..9: note = 03A01 LCDR3
QQYWNSPFT

SEQ ID NO: 59 - protein, 120 aa, synthetic construct
  source 1..120: note = 03A01 VH-D64E VH
DVQFQESGPGLVKPSQSLSLTCTVTGYSITSDFAWNWIRQFPGNELEWMGYISYSGETNYNPSLRSRISITRDTSKNQFFLQLDSVTTEDTATYYCTIRGYYTDSAFVFWGQGTLVTVSA

SEQ ID NO: 60 - protein, 16 aa, synthetic construct
  source 1..16: note = 03A01 VH-D64E HCDR2
YISYSGETNYNPSLRS

SEQ ID NO: 61 - protein, 16 aa, synthetic construct
  source 1..16: note = 03A01 Consensus 1 HCDR2
  VARIANT 7: note = wherein X is D or E
  VARIANT 9: note = wherein X is N or Y
  VARIANT 15: note = wherein X is R or K
YISYSGXTXYNPSLXS

SEQ ID NO: 62 - protein, 16 aa, synthetic construct
  source 1..16: note = 03A01 Consensus 2 HCDR2
  VARIANT 1..16: note = YISYSGDTNYNPSLRS
  VARIANT 1..16: note = YISYSGDTNYNPSLKS
  VARIANT 1..16: note = YISYSGDTYYNPSLKS
YISYSGDTXYNPSLXS

SEQ ID NO: 63 - protein, 120 aa, synthetic construct
  source 1..120: note = Humanized 03A01-VH1
EVQFQESGPGLVKPSQTLSLTCTVTGYSITSDFAWNWIRQPPGKELEWMGYISYSGDTNYNPSLRSRITISRDTSKNQFSLKLSSVTAADTATYYCTIRGYYTDSAFVFWGQGTLVTVSS

SEQ ID NO: 64 - protein, 120 aa, synthetic construct
  source 1..120: note = Humanized 03A01-VH2
EVQFQESGPGLVKPSQTLSLTCTVSGYSITSDFAWNWIRQPPGKGLEWMGYISYSGDTNYNPSLRSRITISRDTSKNQFSLKLSSVTAADTAVYYCTIRGYYTDSAFVFWGQGTLVTVSS

SEQ ID NO: 65 - protein, 120 aa, synthetic construct
  source 1..120: note = Humanized 03A01-VH3
EVQFQESGPGLVKPSQTLSLTCTVSGYSITSDFAWNWIRQPPGKGLEWMGYISYSGDTNYNPSLKSRVTISRDTSKNQFSLKLSSVTAADTAVYYCTIRGYYTDSAFVFWGQGTLVTVSS

SEQ ID NO: 66 - protein, 16 aa, synthetic construct
  source 1..16: note = Humanized 03A01-VH3 HCDR2
YISYSGDTNYNPSLKS

SEQ ID NO: 67 - protein, 120 aa, synthetic construct
  source 1..120: note = Humanized 03A01-VH4
EVQFQESGPGLVKPSQTLSLTCTVSGYSITSDFAWNWIRQPPGKGLEWIGYISYSGDTYYNPSLKSRVTISRDTSKNQFSLKLSSVTAADTAVYYCTIRGYYTDSAFVFWGQGTLVTVSS

SEQ ID NO: 68 - protein, 16 aa, synthetic construct
  source 1..16: note = Humanized 03A01-VH4 HCDR2
YISYSGDTYYNPSLKS

SEQ ID NO: 69 - protein, 107 aa, synthetic construct
  source 1..107: note = Humanized 03A01-VK1
DIQMTQSSSTLSVSLGDRVTITCKASDHINNWLAWYQQKPGKAPRLLISGATSLETGVPSRFSGSGSGLEYTLTISSLQPDDFATYYCQQYWNSPFTFGQGTKLEIK

SEQ ID NO: 70 - protein, 107 aa, synthetic construct
  source 1..107: note = Humanized 03A01-VK2
DIQMTQSPSTLSVSVGDRVTITCKASDHINNWLAWYQQKPGKAPKLLISGATSLETGVPSRFSGSGSGLEYTLTISSLQPDDFATYYCQQYWNSPFTFGQGTKLEIK

SEQ ID NO: 71 - protein, 107 aa, synthetic construct
  source 1..107: note = Humanized 03A01-VK3
DIQMTQSPSTLSASVGDRVTITCKASDHINNWLAWYQQKPGKAPKLLISGATSLETGVPSRFSGSGSGLEYTLTISSLQPDDFATYYCQQYWNSPFTFGQGTKLEIK

SEQ ID NO: 72 - protein, 107 aa, synthetic construct
  source 1..107: note = Humanized 03A01-VK4
DIQMTQSPSTLSVSVGDRVTITCRASQSISSWLAWYQQKPGKAPKLLISGATSLETGVPSRFSGSGSGLEYTLTISSLQPDDFATYYCQQYWNSPFTFGQGTKLEIK

SEQ ID NO: 73 - protein, 11 aa, synthetic construct
  source 1..11: note = Humanized 03A01-VK4 LCDR1
RASQSISSWLA

SEQ ID NO: 74 - protein, 121 aa, synthetic construct
  source 1..121: note = 05F10 VH
EVQLVESGGGLVKPGGSLKLSCAASGFTFSSFAMSWVRQTPAKRLEWVATISSGDLSANYPDTVKGRFTISRDNAKNTLYLQMSSLRSEDTAMYYCTRPPFYFSNDWFAYWGQGTLVTVSA

SEQ ID NO: 75 - protein, 112 aa, synthetic construct
  source 1..112: note = 05F10 VL
DVVMTQTPLSLPVSLEDQASISCRSSQSLEHSNGNTYLHWYLQKPGQSPRLLIYKVSNRFSGVPDRFSGSGSGTDFTLKISRVEAEDLGVYFCSQSTHVPFTFGSGTKLEIK

SEQ ID NO: 76 - protein, 5 aa, synthetic construct
  source 1..5: note = 05F10 HCDR1 - Kabat
SFAMS

SEQ ID NO: 77 - protein, 17 aa, synthetic construct
  source 1..17: note = 05F10 HCDR2 - Kabat
TISSGDLSANYPDTVKG

SEQ ID NO: 78 - protein, 12 aa, synthetic construct
  source 1..12: note = 05F10 HCDR3
PPFYFSNDWFAY

SEQ ID NO: 79 - protein, 16 aa, synthetic construct
  source 1..16: note = 05F10 LCDR1
RSSQSLEHSNGNTYLH

SEQ ID NO: 80 - protein, 7 aa, synthetic construct
  source 1..7: note = 05F10 LCDR2
KVSNRFS

SEQ ID NO: 81 - protein, 9 aa, synthetic construct
  source 1..9: note = 05F10 LCDR3
SQSTHVPFT

SEQ ID NO: 82 - protein, 120 aa, synthetic construct
  source 1..120: note = 11F05 VH
DVQLQESGPGLVKPSQSLSLTCTVTGYSITSDYAWNWIRQFPGNKLEWMGYISYSGSTSYNPSLKSRISITRDTSKNQFFLHLNSVTTEDTATYYCARRGYYSHSAFTYWGQGTLVTVSA

SEQ ID NO: 83 - protein, 107 aa, synthetic construct
  source 1..107: note = 11F05 VL
DIQMTQSSSYLSVSLGGRVTITCKASDHINNWLAWYQQKPGNAPRLLISDATSLETGVPSRFSGSVSGMDYTLSITSLQTEDVATYYCQQYWSTPFTFGSGTKLEIE

SEQ ID NO: 84 - protein, 6 aa, synthetic construct
  source 1..6: note = 11F05 HCDR1
SDYAWN

SEQ ID NO: 85 - protein, 16 aa, synthetic construct
  source 1..16: note = 11F05 HCDR2
YISYSGSTSYNPSLKS

SEQ ID NO: 86 - protein, 11 aa, synthetic construct
  source 1..11: note = 11F05 HCDR3
RGYYSHSAFTY

SEQ ID NO: 87 - protein, 7 aa, synthetic construct
  source 1..7: note = 11F05 LCDR2
DATSLET

SEQ ID NO: 88 - protein, 9 aa, synthetic construct
  source 1..9: note = 11F05 LCDR3
QQYWSTPFT

SEQ ID NO: 89 - protein, 122 aa, synthetic construct
  source 1..122: note = 04D08 VH
EVQLQQSGPELEKPGASVKISCKASGYSFTGYSMNWVKQSNGKSLEWIGNIDPYYGGTSYNQNFKGKATLTVDKSSSTAYMQLKSLTSEDSAVYYCARSYFGNYEDTWFAYWGQGTLVTVSA

SEQ ID NO: 90 - protein, 113 aa, synthetic construct
  source 1..113: note = 04D08 VL
DIVMTQSPSSLTVTAGEKVTMSCKSSQSLLNIGNQNNYLTWYQQKPGQPPKLLIYWASTRESGVPDRFTGSGSGTDFTLTISSVQAEDLAVYYCQNDYSYPLTFGAGTKLELK

SEQ ID NO: 91 - protein, 5 aa, synthetic construct
  source 1..5: note = 04D08 HCDR1
GYSMN

SEQ ID NO: 92 - protein, 17 aa, synthetic construct
  source 1..17: note = 04D08 HCDR2
NIDPYYGGTSYNQNFKG

SEQ ID NO: 93 - protein, 13 aa, synthetic construct
  source 1..13: note = 04D08 HCDR3
SYFGNYEDTWFAY

SEQ ID NO: 94 - protein, 17 aa, synthetic construct
  source 1..17: note = 04D08 LCDR1
KSSQSLLNIGNQNNYLT

SEQ ID NO: 95 - protein, 7 aa, synthetic construct
  source 1..7: note = 04D08 LCDR2
WASTRES

SEQ ID NO: 96 - protein, 9 aa, synthetic construct
  source 1..9: note = 04D08 LCDR3
QNDYSYPLT

SEQ ID NO: 97 - protein, 226 aa, synthetic construct
  source 1..226: note = WT IgG1 Fc Domain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 16 aa, synthetic construct
  source 1..16: note = (GGGS)4 linker
GGGSGGGSGGGSGGGS

SEQ ID NO: 99 - protein, 20 aa, synthetic construct
  source 1..20: note = (GGGGS)4 linker
GGGGSGGGGSGGGGSGGGGS

SEQ ID NO: 100 - protein, 19 aa, synthetic construct
  source 1..19: note = GGGS-(GGGGS)3
GGGSGGGGSGGGGSGGGGS

SEQ ID NO: 101 - protein, 20 aa, synthetic construct
  source 1..20: note = (GS)10
GSGSGSGSGSGSGSGSGSGS

SEQ ID NO: 102 - protein, 30 aa, synthetic construct
  source 1..30: note = (GGS)10
GGSGGSGGSGGSGGSGGSGGSGGSGGSGGS

SEQ ID NO: 103 - protein, 40 aa, synthetic construct
  source 1..40: note = (GGGS)10
GGGSGGGSGGGSGGGSGGGSGGGSGGGSGGGSGGGSGGGS

SEQ ID NO: 104 - protein, 40 aa, synthetic construct
  source 1..40: note = (GGSG)10
GGSGGGSGGGSGGGSGGGSGGGSGGGSGGGSGGGSGGGSG

SEQ ID NO: 105 - protein, 50 aa, synthetic construct
  source 1..50: note = (GGSGG)10
GGSGGGGSGGGGSGGGGSGGGGSGGGGSGGGGSGGGGSGGGGSGGGGSGG

SEQ ID NO: 106 - protein, 50 aa, synthetic construct
  source 1..50: note = (GGGGS)10
GGGGSGGGGSGGGGSGGGGSGGGGSGGGGSGGGGSGGGGSGGGGSGGGGS

SEQ ID NO: 107 - protein, 15 aa, synthetic construct
  source 1..15: note = (GGGGS)3
GGGGSGGGGSGGGGS

SEQ ID NO: 108 - protein, 100 aa, synthetic construct
  source 1..100: note = (GGGGS)20
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 109 - protein, 100 aa, synthetic construct
  source 1..100: note = (GGSGG)20
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 110 - protein, 133 aa, synthetic construct [sequence omitted]

SEQ ID NO: 111 - protein, 223 aa, synthetic construct
  source 1..223: note = HC: HC-02(VH6) (02A01 VH6, human CH1)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 112 - protein, 371 aa, synthetic construct
  source 1..371: note = LC: LC-02-VK3-LM-IL2 (D30E, N35Q)
EIVLTQSPATLSLSPGERATISCRASESVESYDIQSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 113 - protein, 450 aa, synthetic construct
  source 1..450: note = HC1: HC(k)-02(VH6)TW. LA.LA.NA (02A01 VH6, human CH1, CH2-CH3)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 227 aa, synthetic construct
  source 1..227: note = HC2: HC(h)-CH2-CH3-TS.LA.YV.LA.LA.NA (human CH2-CH3)
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 115 - protein, 450 aa, synthetic construct
  source 1..450: note = HC: HC-02(VH6) LA.LA.NA (02A01 VH6, human CH1, CH2-CH3)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 603 aa, synthetic construct
  source 1..603: note = HC: HC-02(VH6) LA.LA.NA-IL2 (02A01 VH6, human CH1, CH2-CH3-IL2)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 117 - protein, 218 aa, synthetic construct
  source 1..218: note = LC: LC-02-VK3-LM
EIVLTQSPATLSLSPGERATISCRASESVESYDIQSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 603 aa, synthetic construct
  source 1..603: note = HC1: HC(k)-02(VH6)TW. LA.LA.NA-IL2 (02A01 VH6, human CH1, CH2-CH3-IL2)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 119 - protein, 450 aa, synthetic construct
  source 1..450: note = HC2: HC(h)-02(VH6)TS.LA.YV.LA.LA.NA (02A01 VH6, human CH1, CH2-CH3)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 120 - protein, 603 aa, synthetic construct
  source 1..603: note = HC: IL2-HC-02(VH6) LA.LA.NA (IL2-02A01 VH6, human CH1, CH2-CH3)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSEVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 371 aa, synthetic construct
  source 1..371: note = LC: IL2-LC-02-VK3-LM (D30E, N35Q)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSEIVLTQSPATLSLSPGERATISCRASESVESYDIQSMHWYQQKPGQAPRLLIYRASHLQSGIPARFSGSGSRTDFTLTISSLEPEDFATYYCQQSNEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 603 aa, synthetic construct
  source 1..603: note = HC2: IL2-HC(h)-02(VH6)TS.LA.YV.LA.LA.NA (IL2-02A01 VH6, human CH1, CH2-CH3)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSEVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 123 - protein, 376 aa, synthetic construct
  source 1..376: note = HC: HC-02(VH6)-IL2 (02A01 VH6, human CH1, IL2)
EVQLVQSGAEVKKPGATVKISCKASGYTFTDFTMHWVQQAPGKGLEWIGLVNPNNGGNIYAEKFQGRVTLTVDTSTSTAYMELSSLRSEDTAVYYCGRVPLYTSHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 124 - protein, 380 aa, synthetic construct
  source 1..380: note = HC2: HC(h)-CH2-CH3-TS.LA.YV.LA.LA.NA-IL2 (human CH2-CH3-IL2)
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 125 - protein, 227 aa, synthetic construct
  source 1..227: note = HC(h)-TS.LA.YV.LA.LA.PG.NA
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 126 - protein, 223 aa, synthetic construct
  source 1..223: note = RSVFabVH
QVTLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 127 - protein, 450 aa, synthetic construct
  source 1..450: note = RSV-HC(k)-TW LA.LA.PG.NA
QVTLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 450 aa, synthetic construct
  source 1..450: note = VH-RSV-2aLALAPGNA
QVTLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 129 - protein, 366 aa, synthetic construct
  source 1..366: note = RSVFab-IL2LC
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 130 - protein, 366 aa, synthetic construct; identical to SEQ ID NO: 129
  source 1..366: note = RSVFab-IL2LC

SEQ ID NO: 131 - protein, 366 aa, synthetic construct
  source 1..366: note = LC-RSV-mK- hIL2
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 132 - protein, 450 aa, synthetic construct
  source 1..450: note = RSVHC-LALAPGNA
QVTLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 366 aa, synthetic construct; identical to SEQ ID NO: 129
  source 1..366: note = RSVFab-IL2LC

SEQ ID NO: 134 - protein, 602 aa, synthetic construct
  source 1..602: note = HC(k)-RSV-TW-(IL2)
QVTLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 135 - protein, 450 aa, synthetic construct
  source 1..450: note = HC(h)- RSV-TS.LA.YV.LA.LA.PG.NA
QVTLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALGAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 136 - protein, 218 aa, synthetic construct
  source 1..218: note = RSV-VL-hKappa
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 161 aa, synthetic construct
  source 1..161: note = CD8 protein fragment (AA 22-182 of WT CD8 NP_001139345.1)
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 138 - protein, 235 aa, synthetic construct
  source 1..235: note = CD NP_001139345.1
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 139 - protein, 7 aa, synthetic construct
  source 1..7: note = 02A01 HCDR1 - Chothia
GYTFTDF

SEQ ID NO: 140 - protein, 6 aa, synthetic construct
  source 1..6: note = 02A01 HCDR2 - Chothia
NPNNGG

SEQ ID NO: 141 - protein, 7 aa, synthetic construct
  source 1..7: note = Humanized 02A01-VH5 HCDR1 - Chothia
GYTFTDY

SEQ ID NO: 142 - protein, 8 aa, synthetic construct
  source 1..8: note = 03A01 HCDR1 - Chothia
GYSITSDF

SEQ ID NO: 143 - protein, 5 aa, synthetic construct
  source 1..5: note = 03A01 HCDR2 - Chothia
SYSGD

SEQ ID NO: 144 - protein, 5 aa, synthetic construct
  source 1..5: note = 03A01 HCDR2 - 03A01 VH-D64E - Chothia
SYSGE

SEQ ID NO: 145 - protein, 5 aa, synthetic construct
  source 1..5: note = 03A01 HCDR2 consensus - Chothia
  VARIANT 5: note = wherein X is D or E
SYSGX

SEQ ID NO: 146 - protein, 7 aa, synthetic construct
  source 1..7: note = 05F10 HCDR1 - Chothia
GFTFSSF

SEQ ID NO: 147 - protein, 6 aa, synthetic construct
  source 1..6: note = 05F10 HCDR2 - Chothia
SSGDLS

SEQ ID NO: 148 - protein, 8 aa, synthetic construct
  source 1..8: note = 11F05 HCDR1 - Chothia
GYSITSDY

SEQ ID NO: 149 - protein, 5 aa, synthetic construct
  source 1..5: note = 11F05 HCDR2 - Chothia
SYSGS

SEQ ID NO: 150 - protein, 7 aa, synthetic construct
  source 1..7: note = 04D08 HCDR1 - Chothia
GYSFTGY

SEQ ID NO: 151 - protein, 6 aa, synthetic construct
  source 1..6: note = 04D08 HCDR2 - Chothia
DPYYGG